(19) United States
(12) Patent Application Publication
(10) Pub. No.: US 2026/0070952 A1
(43) Pub. Date: Mar. 12, 2026

(54) PRODUCTION OF LEGHEMOGLOBIN IN PLANTS

(71) Applicant: PIONEER HI-BRED INTERNATIONAL, INC., JOHNSTON, IA (US)

(72) Inventors: JOHN D EVERARD, GRIMES, IA (US); ANTHONY J KINNEY, WILMINGTON, DE (US); ZHAN-BIN LIU, CLIVE, IA (US); KNUT MEYER, WILMINGTON, DE (US); KEVIN G RIPP, DES MOINES, IA (US)

(73) Assignee: PIONEER HI-BRED INTERNATIONAL, INC., JOHNSTON, IA (US)

(21) Appl. No.: 19/106,400

(22) PCT Filed: Jul. 26, 2023
(86) PCT No.: PCT/US23/71018

§ 371 Date: Feb. 25, 2025

Related U.S. Application Data
(60) Provisional application No. 63/369,697, filed on Jul. 28, 2022.

(51) Int. Cl. C07K 14/415; C12N 9/02; C12N 9/10; C12N 9/88; C12N 9/90; C12N 15/52; C12N 15/82

(52) U.S. Cl. CPC C07K 14/415; C12N 9/0008; C12N 9/001; C12N 9/1085; C12N 9/88; C12N 9/90; C12N 15/52; C12N 15/8243; C12Y 102/0107; C12Y 103/03004; C12Y 205/01061; C12Y 402/01024; C12Y 499/01001; C12Y 504/03008

(57) ABSTRACT

Plants and plant tissue such as leaves comprising leghemoglobin are produced by modifying the genome of the plant or introducing leghemoglobin coding or regulatory sequences into the plant. Plants, leaves, fruits, tubers, roots, seeds, and protein compositions comprising leghemoglobin are provided. Protein compositions comprising leghemoglobin, such as isolates and concentrates, can be made from the plants, tissue, fruits, tubers, roots, seeds and leaves. Additionally, methods for generating and using plants, tissue, fruits, tubers, roots, seeds, leaves and protein compositions comprising leghemoglobin are disclosed.

### REFERENCE TO SEQUENCE LISTING SUBMITTED ELECTRONICALLY

The official copy of the sequence listing is submitted electronically via Patent Center as an XML formatted sequence listing with a file named 108291_SequenceListing created on 25 Jul. 2023 and having a size of 180,902 bytes and is filed concurrently with the specification. The sequence listing comprised in this XML formatted document is part of the specification and is herein incorporated by reference in its entirety.

### BACKGROUND

Soy legume hemoglobin, or leghemoglobin, is a globin protein found in the nitrogen-fixing root nodules of leguminous plants. Leghemoglobin carries heme, an iron-containing molecule, and functions to protect the nitrogenase enzyme from oxygen inactivation and to facilitate oxygen flow to the nitrogen-fixing bacteria. Leghemoglobin can be fermented from engineered yeast and has use in meat replacements by mimicking the flavor contributed by hemoglobin in meat. Animal-based meat replacement with plant-based proteins, such as leghemoglobin, is becoming an industrial trend in food applications. Accordingly, there is a need to develop compositions and methods to increase the production of leghemoglobin in plants for food applications. This disclosure provides such compositions and methods.

### SUMMARY

Disclosed are plant leaves, roots, seeds, fruits, flowers, and vegetative tissue comprising a genomic modification and a leghemoglobin protein expressed in the plant leaves, the genomic modification comprising one or more of (i) an insertion of a leghemoglobin coding sequence into a native non-leghemoglobin gene such that the leghemoglobin coding sequence replaces all or part of the native non-leghemoglobin gene coding sequence, (ii) an insertion of a heterologous regulatory enhancer or heterologous promotor sequence operably linked to a native leghemoglobin coding sequence and (iii) a substitution or deletion wherein the substitution or deletion creates or enhances a regulatory enhancer or promotor sequence of a native leghemoglobin coding sequence. The native non-leghemoglobin gene can one or more of a RUBISCO gene, a vegetative storage protein, a RUBISCO Activase gene, or any combination thereof.

In some embodiments, the plant leaf, root, seed, fruit, flower, or vegetative tissue can further comprise one or more porphyrin pathway genes operably linked to a heterologous regulatory element, which genes can be contained in a recombinant construct, or the leaf genome can have nucleotide insertions, deletions, or substitutions to such that the one or more porphyrin pathway genes are operably linked to a heterologous regulatory element. The porphyrin pathway gene can be a glutamyl-tRNA reductase, a ferrochelatase, a glutamate-1-semialdehyde 2, a 1-aminomutase, an aminolevulinate dehydratase, a hydroxymethylbilane synthase, a urophorphyrinogen III synthase, a urophorphyrinogen decarboxylase, a coporphyrinogen III oxidase, and a protoporphyrinogen oxidase, or any combination thereof.

Disclosed are plant leaves, roots, seeds, fruits, flowers, and vegetative tissue comprising a leghemoglobin protein in an amount of at least 0.1% of total protein in the plant leaf, the plant leaf comprising a leghemoglobin coding sequence operably linked to a heterologous regulatory element (e.g., a promoter, such as a leaf preferred promoter), such as contained in a recombinant construct.

In some embodiments, the plant leaves, roots, seeds, fruits, flowers, and vegetative tissue disclosed herein further comprise a sequence targeting the leghemoglobin to an intracellular compartment, such as a plastid or endoplasmic reticulum, operably linked to the leghemoglobin coding sequence.

In some embodiments, the plant leaves, roots, seeds, fruits, flowers, and vegetative tissue disclosed herein have the genome modified to introduce an insertion, deletion, or substitution into a native leghemoglobin gene, such as an insertion of a heterologous regulatory enhancer or heterologous promoter sequence operably linked to the native leghemoglobin coding sequence, or a substitution wherein the substitution creates or enhances a native leghemoglobin coding sequence regulatory enhancer or a native leghemoglobin coding sequence promotor sequence. In some embodiments, the plant leaves, roots, seeds, fruits, flowers, and vegetative tissue disclosed herein have been modified to replace all or part of a coding sequence of a non-leghemoglobin gene, such as a RUBISCO gene, a RUBISCO Activase gene, or a vegetative storage protein gene, with a leghemoglobin coding sequence.

In some embodiments, the plant genome is modified to introduce an insertion, and the insertion comprises a targeting sequence operably linked to the leghemoglobin coding sequence which targets the leghemoglobin to an intracellular compartment, such as the plastid or endoplasmic reticulum.

In some embodiments, the plant leaves, roots, seeds, fruits, flowers, and vegetative tissue, such as a transgenic leaf, express leghemoglobin, for example in an amount of at least 0.1% of the total leaf protein and do not contain a modification to a porphyrin pathway gene, such as for example, do not contain any or all of (i) a recombinant construct comprising a sequence encoding a glutamyl tRNA reductase, or a truncated portion thereof, (ii) a recombinant construct comprising a sequence encoding a ferrochelatase, (iii) a recombinant construct comprising a sequence encoding a glutamyl tRNA reductase binding protein, and (iv) a recombinant construct comprising a sequence encoding an aminolevulinic acid synthase.

In some embodiments, the plant leaves, roots, seeds, fruits, flowers, and vegetative tissue are from a soybean plant, a pea plant, a maize plant, an alfalfa plant, or a rice plant.

Provided are plants comprising one or more of the modified plant leaves, roots, seeds, fruits, flowers, and vegetative tissue described herein. In some embodiments, the plant comprising one or more modified plant leaves, roots, fruits, flowers, and vegetative tissue can further comprise a seed comprising a leghemoglobin protein in an amount of at least 0.5% of the total protein in the seed. The plant or seed can include a modification to replace all or part of a seed storage protein gene with a leghemoglobin gene coding sequence and/or an additional modification to enhance seed protein content, such as in a CCT-domain containing protein, a reticulon, a trehalose phosphate synthase, a HECT ubiquitin Ligase, a MFT (mother of flowering) polypeptide, and/or a raffinose synthase. In some embodiments, the plant comprising one or more modified plant leaves, seeds, fruits, flowers, and vegetative tissue can further comprise below-ground vegetative tissue comprising a leghemoglobin protein in an amount of at least 0.5% of the total protein in the below-ground vegetative tissue.

In some embodiments, a plant expressing leghemoglobin protein in one or more of its leaves, roots, seeds, fruits, flowers, and vegetative tissue in an amount of at least 0.1% of total protein, comprises a leghemoglobin coding sequence operably linked to a heterologous element, wherein the leaf does not contain one or all of a recombinant construct comprising a sequence encoding a glutamyl tRNA reductase, or a truncated portion thereof, a recombinant construct comprising a sequence encoding a ferrochelatase, a recombinant construct comprising a sequence encoding a glutamyl tRNA reductase binding protein, and a recombinant construct comprising a sequence encoding an aminolevulinic acid synthase.

In some embodiments, the plant genome is modified to introduce an insertion, deletion, or substitution into a native leghemoglobin gene such as an insertion of a heterologous regulatory enhancer or heterologous promoter sequence operably linked to the native leghemoglobin coding sequence, or a substitution creating or enhancing a native leghemoglobin coding sequence regulatory enhancer or a native leghemoglobin coding sequence promotor sequence.

In some embodiments, the leghemoglobin is expressed or present in one or more of the plant leaves, roots, seeds, fruits, flowers, and vegetative tissue described herein in a heme-loaded form, such that the leghemoglobin is in a complex which includes the leghemoglobin protein associated with a heme group (porphyrin bound to iron). Such a complex may be observed as a pink or red color or may be measured using in vitro techniques.

Provided are methods for extracting leghemoglobin protein from the plant leaves, roots, seeds, fruits, flowers, and vegetative tissue described herein, in which one or more of the leaves, roots, seeds, fruits, flowers, and vegetative tissue are contacted with at least one of a cellulase, a hemicellulase, and a pectinase under conditions sufficient to degrade the polysaccharides in the leaves, roots, seeds, fruits, flowers, and vegetative tissue and filtering the permeant from the residue.

Provided are extracts, fractions and isolates produced from the plant leaves, roots, seeds, fruits, flowers, and vegetative tissue described herein. The isolate can contain at least 0.2% leghemoglobin by weight of total protein, and at least about 50% of the leghemoglobin can be hemelated with an iron group. The protein fraction extracted from the plant leaves, roots, seeds, fruits, flowers, and vegetative tissue described herein, can comprise at least 0.1% leghemoglobin by weight of total protein.

### BRIEF DESCRIPTION OF THE DRAWINGS AND THE SEQUENCE LISTING

The disclosure can be more fully understood from the following detailed description and the accompanying drawings and Sequence Listing, which form a part of this application.

FIG. 1 is a schematic showing genome engineering of the leghemoglobin gene into the native soybean ribulose-1,5-bisphosphate carboxylase oxygenase small subunit (RUBISCO SSU) gene locus by RUBISCO-CR1/CR2 gRNA pair.

FIG. 2 is a schematic showing genome engineering of the leghemoglobin gene into the native soybean vegetative storage protein (VSP) gene locus by VSP-CR1/CR2 gRNA pair.

FIG. 3 is a schematic showing genome engineering of the leghemoglobin gene into the native soybean RUBISCO Activase (RCA) gene locus by RCA-CR1/CR2 gRNA pair.

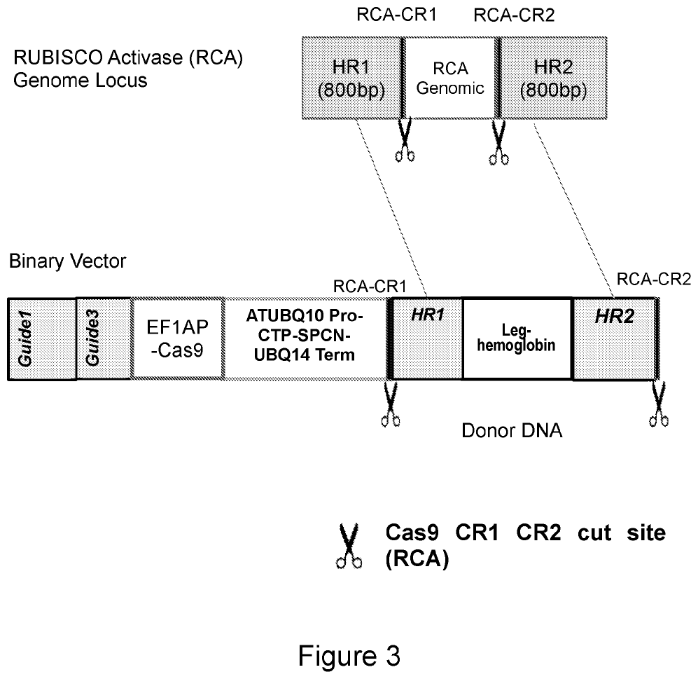

The sequence descriptions (Table 1) and sequence listing attached hereto comply with the rules governing nucleotide and amino acid sequence disclosures in patent applications as set forth in 37 C.F.R. §§ 1.831 and 1.835.

TABLE 1
Sequence Listing Description

| SEQ | | |
|---|---|---|
| ID | | |
| NO: | Name | Type |
| 1 | Glyma.20g191200 Leghemoglobin CDS | DNA |
| 2 | Glyma.20g191200 Leghemoglobin peptide | PRT |
| 3 | Glyma.20g191200 leghemoglobin genomic sequence | DNA |
| 4 | Rubisco SSUSP::Leghemoglobin | DNA |
| 5 | Rubisco SSUSP::leghemoglobin | PRT |
| 6 | Leghemoglobin-KDEL | DNA |
| 7 | Leghemoglobin-KDEL | PRT |
| 8 | RUBISCO SSU promoter (including 5′UTR) | DNA |
| 9 | RCA2 promoter (including 5′UTR) | DNA |
| 10 | Glyma.04g0898000 CDS | DNA |
| 11 | Glyma.04g0898000 peptide | PRT |
| 12 | Glyma.04g050400 CDS | DNA |
| 13 | Glyma.04g050400 Peptide | PRT |
| 14 | Ubiquitin promoter (including 5′UTR and intron1) | DNA |
| 15 | EF1A Promoter (including 5′UTR and intron1) | DNA |
| 16 | GM-RUBISCO-CR1 | RNA |
| 17 | GM-RUBISCO-CR2 | RNA |
| 18 | GM-VSP-CR1 | RNA |
| 19 | GM-VSP-CR2 | RNA |
| 20 | GM-RCA-CR1 | RNA |
| 21 | GM-RCA-CR2 | RNA |
| 22 | glyma.13g046200 RUBISCO SSU genomic sequences | DNA |
| 23 | glyma.13g046200 RUBISCO SSU peptide | PRT |
| 24 | glyma.19g046800 RUBISCO SSU genomic sequences | DNA |
| 25 | glyma.19g046800 RUBISCO SSU peptide | PRT |
| 26 | glyma.07g014500 VSP-A genomic sequences | DNA |
| 27 | glyma.07g014500 VSP-A peptide | PRT |
| 28 | glyma.08g200100 VSP-B genomic sequences | DNA |
| 29 | glyma.08g200100 VSP-B peptide | PRT |
| 30 | glyma.08g200200 VSP1 genomic sequences | DNA |
| 31 | glyma.08g200200 VSP1 peptide | PRT |
| 32 | glyma.15g218400 VSP2 genomic sequences | DNA |
| 33 | glyma.15g218400 VSP2 peptide | PRT |
| 34 | glyma.08g200000 VSP3 genomic sequences | DNA |
| 35 | glyma.08g200000 VSP3 peptide | PRT |
| 36 | glyma.11g221000 RUBISCO activase RCA2 genomic seq | DNA |
| 37 | glyma.11g221000 RUBISCO activase RCA2 peptide | PRT |
| 38 | glyma.02g249600 RUBISCO activase RCA genomic seq | DNA |
| 39 | glyma.02g249600 RUBISCO activase RCA peptide | PRT |
| 40 | glyma.14g067000 RUBISCO activase RCA genomic seq | DNA |
| 41 | glyma.14g067000 RUBISCO activase RCA peptide | PRT |
| 42 | Donor DNA for GM-RUbisco-CR1/CR2 design | |
| 43 | Donor DNA for GM-VSP-CR1/CR2 design | DNA |
| 44 | Donor DNA for GM-RCA-CR1/CR1 design | DNA |
| 45 | Globulin peptide | PRT |
| 46 | Globulin peptide | PRT |
| 47 | Glyma.04G037000.1 CDS urophorphyrinogen III synthase | DNA |
| 48 | Glyma.04G037000.1 polypeptide urophorphyrinogen III | PRT |
| | synthase | |
| 49 | glutamate-1-semialdehyde 2,1-aminomutase | DNA |
| | Glyma.04G002900.1 | |
| 50 | glutamate-1-semialdehyde 2,1-aminomutase | PRT |
| | Glyma.04G002900.1 | |
| 51 | Glutamyl-tRNA reductase-binding protein | DNA |
| | Glyma.08G222600 | |
| 52 | Glutamyl-tRNA reductase-binding protein | PRT |
| | Glyma.08G222600 | |
| 53 | RUBISCO SSUSP | PRT |
| 54 | Endoplasmic reticulum (ER) targeting peptide | PRT |

### DETAILED DESCRIPTION

The present disclosure provides compositions and methods for producing plant tissue such as leaves expressing the leghemoglobin protein, the leghemoglobin complex or a combination thereof. Leghemoglobin is a protein synthesized in soy root nodules upon colonization by nitrogen-fixing bacteria. As used herein, “leghemoglobin protein” or “leghemoglobin” refer to the globulin protein or polypeptide, whether unfolded or folded into a monomer and which may or may not have associated with it a heme group (porphyrin bound to iron). As used herein “leghemoglobin complex” or “leghemoglobin protein complex” refers particularly to the complex which includes the leghemoglobin protein associated with a heme group (porphyrin bound to iron). Such a complex, when present in sufficient quantities can impart a red or pink color to the cells or tissue containing the complex, detectable to the eye. As used herein with respect to the color of a plant or leaf in the transverse section, pink color means any shade of pink or red. The pink color of the plant tissue may be viewable, for example, under a microscope or through use of a camera and may be calculated after subtracting absorbance by chlorophyll for example.

Accordingly, one aspect of the disclosure provides plant tissue such as leaves comprising a leghemoglobin protein (e.g., the leghemoglobin without a heme group, the leghemoglobin complex, or a combination of both forms) in an amount of at least 0.01%, 0.05%, 0.5%, 1%, 1.5%, 2%, 2.5%, 3%, 4%, 5%, 6%, 7%, 8%, 9%, 10% or more and less than 75%, 50%, 25%, 20%, 15%, 10%, 5%, 4%, or 3% of the total leaf protein. In certain embodiments, at least 5, 10, 15, 20, 25, 30, 40, 50, 60, 70, 80, 90 or 95 percent and less than 100, 99.9, 95, 90, 85, 80, 70, 60 or 50 percent of the total leghemoglobin forms a complex with a heme group in the plant leaf. In certain embodiments, the plant leaf comprises a leghemoglobin coding sequence operably linked to a heterologous regulatory element.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

As used herein, “heterologous” in reference to a sequence is a sequence that originates from a foreign species, or, if from the same species, is substantially modified from its native form in composition and/or genomic locus by deliberate human intervention. For example, a heterologous regulatory element operably linked to a polynucleotide that is from a species different from the species from which the polynucleotide was derived, or, if from the same/analogous species, one or both are substantially modified from their original form and/or genomic locus, or the regulatory element is not the native promoter for the operably linked polynucleotide.

In certain embodiments, the plant leaf comprises a recombinant construct comprising a leghemoglobin coding sequence operably linked to the heterologous regulatory element. In certain embodiments, the heterologous regulatory element is a promoter. In certain embodiments, the promoter is a leaf preferred promoter.

The use of the term “recombinant constructs” herein is not intended to limit the embodiments to nucleotide constructs comprising DNA. Those of ordinary skill in the art will recognize that recombinant constructs, particularly polynucleotides and oligonucleotides composed of ribonucleotides and combinations of ribonucleotides and deoxyribonucleotides, may also be employed in the methods disclosed herein. The recombinant constructs, nucleic acids, and nucleotide sequences of the embodiments additionally encompass all complementary forms of such constructs, molecules, and sequences. Further, the recombinant constructs, nucleotide molecules, and nucleotide sequences of the embodiments encompass all nucleotide constructs, molecules, and sequences which can be employed in the methods of the embodiments for transforming plants and/or plant tissue such as leaves including, but not limited to, those comprised of deoxyribonucleotides, ribonucleotides, and combinations thereof. Such deoxyribonucleotides and ribonucleotides include both naturally occurring molecules and synthetic analogues. The recombinant constructs, nucleic acids, and nucleotide sequences of the embodiments also encompass all forms of nucleotide constructs including, but not limited to, single-stranded forms, double-stranded forms, hairpins, stem-and-loop structures and the like.

In certain embodiments, the leghemoglobin polynucleotides (e.g., leghemoglobin coding sequence) described herein are provided in expression cassettes (e.g., a plasmid, cosmid, virus, autonomously replicating sequence, phage, or linear or circular single-stranded or double-stranded DNA or RNA nucleotide sequence) for expression in a plant of interest or any organism of interest. The cassette can include 5′ and 3′ regulatory sequences operably linked to a leghemoglobin coding sequence polynucleotide. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the leghemoglobin coding sequence to be under the transcriptional regulation of the regulatory regions. The expression cassette may additionally contain selectable marker genes.

The expression cassette can include in the 5′-3′ direction of transcription, a transcriptional and translational initiation region (e.g., a promoter), a leghemoglobin coding sequence, and a transcriptional and translational termination region (e.g., termination region) functional in plants. The regulatory regions (e.g., promoters, transcriptional regulatory regions, and translational termination regions) and/or the leghemoglobin coding sequence may be native/analogous to the host cell or to each other. Alternatively, the regulatory regions and/or the leghemoglobin coding sequence may be heterologous to the host cell or to each other.

The termination region may be native with the transcriptional initiation region, with the plant host, or may be derived from another source (i.e., foreign or heterologous) than the promoter, the leghemoglobin coding sequence, the plant host, or any combination thereof.

The expression cassette may additionally contain a 5′ leader sequences. Such leader sequences can act to enhance translation. Translation leaders are known in the art and include viral translational leader sequences.

Generally, the expression cassette can comprise a selectable marker gene for the selection of transformed cells. Selectable marker genes are utilized for the selection of transformed cells or tissues. Marker genes include without limitation genes encoding antibiotic resistance, such as those encoding neomycin phosphotransferase II (NEO) and hygromycin phosphotransferase (HPT), as well as genes conferring resistance to herbicidal compounds, such as glyphosate, glufosinate ammonium, bromoxynil, imidazolinones, and 2,4-dichlorophenoxyacetate (2,4-D). The above list of selectable marker genes is not meant to be limiting. Any selectable marker gene can be used in the present disclosure.

In preparing the expression cassette, the various DNA fragments may be manipulated, to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

In certain embodiments of the methods and compositions described herein, the nucleic acid construct or expression cassette is introduced and expressed in a plant or plant leaf. The recombinant constructs or expression cassettes disclosed herein may be used for transformation of any plant species.

“Introducing”, “introduced” or the like is intended to mean presenting to the plant, plant cell, seed, and/or grain the inventive polynucleotide or resulting polypeptide in such a manner that the sequence gains access to the interior of a cell of the plant. The methods of the disclosure do not depend on a particular method for introducing a sequence into a plant, plant cell, seed, and/or grain, only that the polynucleotide or polypeptide gains access to the interior of at least one cell of the plant.

As used herein “operably linked” is intended to mean a functional linkage between two or more elements. For, example, an operable linkage between a polynucleotide of interest and a regulatory sequence (e.g., a promoter) is a functional link that allows for expression of the polynucleotide of interest. Operably linked elements may be contiguous or non-contiguous. When used to refer to the joining of two protein coding regions, operably linked is intended that the coding regions are in the same reading frame.

As used herein “regulatory element” generally refers to a transcriptional regulatory element involved in regulating the transcription of a nucleic acid molecule such as a gene or a target gene. The regulatory element is a nucleic acid and may include a promoter, an enhancer, an intron, expression modulating elements (EMEs), a 5′-untranslated region (5′-UTR, also known as a leader sequence), or a 3′-UTR or a combination thereof. A regulatory element may act in “cis” or “trans”, and generally it acts in “cis”, i.e., it activates expression of genes located on the same nucleic acid molecule, e.g., a chromosome, where the regulatory element is located.

An “enhancer” element is any nucleic acid molecule that increases transcription of a nucleic acid molecule when functionally linked to a promoter regardless of its relative position. Various enhancers are known in the art including for example, introns with gene expression enhancing properties in plants, the ubiquitin intron (i.e., the maize ubiquitin intron 1 (see, for example, NCBI sequence S94464)), the omega enhancer or the omega prime enhancer (Gallie, et al., (1989) Molecular Biology of RNA ed. Cech(Liss, New York) 237-256 and Gallic, et al., (1987) Gene 60:217-25), the CaMV 35S enhancer (see, e.g., Benfey, et al., (1990) EMBO J. 9:1685-96) and the enhancers of U.S. Pat. No. 7,803,992 may also be used. The above list of transcriptional enhancers is not meant to be limiting. Any appropriate transcriptional enhancer can be used in the embodiments described herein.

A “repressor” (also sometimes called herein silencer) is defined as any nucleic acid molecule which inhibits the transcription when functionally linked to a promoter regardless of relative position. The term “cis-element” generally refers to transcriptional regulatory element that affects or modulates expression of an operably linked transcribable polynucleotide, where the transcribable polynucleotide is present in the same DNA sequence. A cis-element may function to bind transcription factors, which are trans-acting polypeptides that regulate transcription.

An “intron” is an intervening sequence in a gene that is transcribed into RNA but is then excised in the process of generating the mature mRNA. The term is also used for the excised RNA sequences. An “exon” is a portion of the sequence of a gene that is transcribed and is found in the mature mRNA derived from the gene but is not necessarily a part of the sequence that encodes the final gene product. The 5′ untranslated region (5′UTR) (also known as a translational leader sequence or leader RNA) is the region of an mRNA that is directly upstream from the initiation codon. This region is involved in the regulation of translation of a transcript by differing mechanisms in viruses, prokaryotes and eukaryotes. The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

As used herein “promoter” refers to a region of DNA upstream from the start of transcription and involved in recognition and binding of RNA polymerase and other proteins to initiate transcription. A “plant promoter” is a promoter capable of initiating transcription in plant cells. In certain embodiments, the polynucleotides described herein are operably linked to a promoter that drives expression in a plant cell. Any promoter known in the art can be used in the methods of the present disclosure including, but not limited to, constitutive promoters, pathogen-inducible promoters, wound-inducible promoters, tissue-preferred promoters, and chemical-regulated promoters. The choice of promoter may depend on the desired timing and location of expression in the transformed plant as well as other factors, which are known to those of skill in the art. Such constitutive promoters include, for example, the core promoter of the Rsyn7 promoter and other constitutive promoters disclosed in WO 99/43838 and U.S. Pat. No. 6,072,050; the core CaMV 35S promoter; rice actin; ubiquitin; pEMU; MAS; ALS; and the like. Other constitutive promoters include, for example, those disclosed in U.S. Pat. Nos. 5,608,149; 5,608,144; 5,604,121; 5,569,597; 5,466,785; 5,399,680; 5,268,463; 5,608,142; and 6,177,611, which are known in the art, and can be contemplated for use in the present disclosure.

Generally, it can be beneficial to express the gene from an inducible promoter, particularly from a pathogen-inducible promoter. Such promoters include those from pathogenesis-related proteins (PR proteins), which are induced following infection by a pathogen, e.g., PR proteins, SAR proteins, beta-1,3-glucanase, chitinase, etc.

Of interest are promoters that are expressed locally at or near the site of pathogen infection. Additionally, as pathogens find entry into plants through wounds or insect damage, a wound-inducible promoter can be used in the constructions of the disclosure. Such wound-inducible promoters include potato proteinase inhibitor (pin II) gene, wun1 and wun2, win1 and win2, systemin, WIP1, MPI gene, and the like.

Chemical-regulated promoters can be used to modulate the expression of a gene in a plant through the application of an exogenous chemical regulator. Depending upon the objective, the promoter can be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (e.g., the glucocorticoid-inducible promoter, and tetracycline-inducible and tetracycline-repressible promoters).

Tissue-preferred promoters can be utilized to target enhanced expression of the target genes or proteins within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al. (1997) Plant J. 12(2): 255-265; Kawamata et al. (1997) Plant Cell Physiol. 38(7): 792-803; Hansen et al. (1997) Mol. Gen Genet. 254(3): 337-343; Russell et al. (1997) Transgenic Res. 6(2): 157-168; Rinehart et al. (1996) Plant Physiol. 112(3): 1331-1341; Van Camp et al. (1996) Plant Physiol. 112(2): 525-535; Canevascini et al. (1996) Plant Physiol. 112(2): 513-524; Yamamoto et al. (1994) Plant Cell Physiol. 35(5): 773-778; Lam (1994) Results Probl. Cell Differ. 20:181-196; Orozco et al. (1993) Plant Mol Biol. 23(6): 1129-1138; Matsuoka et al. (1993) Proc Natl. Acad. Sci. USA 90(20): 9586-9590; and Guevara-Garcia et al. (1993) Plant J. 4(3): 495-505. Such promoters can be modified.

Leaf-preferred promoters are well known in the art. For example, the tobacco ferredoxin-binding subunit of photosystem 1 psaDb promoter (Yamamoto et al. (1997), Plant J. 12(2) 255-265); the Arabidopsis glyceraldehyde 3-phosphate dehydrogenase gene promoter (Kwon et al. (1994) Plant Physiol. 105:357-67); the pine chlorophyll a/b binding protein of PSII cab-6 promoter (Yamamoto et al. (1994) Plant Cell Physiol. 35(5): 773-778); the wheat chlorophyll a/b binding protein of PSII cab-1 promoter (Gotor et al. (1993) Plant J. 3:509-18); the spinach ribulose bisphosphate carboxylase/oxygenase activase promoter (Orozco et al. (1993) Plant Mol. Biol. 23(6): 1129-1138); and the maize PPDK promoter (Matsuoka et al. (1993) Proc. Natl. Acad. Sci. USA 90(20):9586-9590).

“Seed-preferred” promoters include both “seed-specific” promoters (those promoters active during seed development such as promoters of seed storage proteins) as well as “seed-germinating” promoters (those promoters active during seed germination). Such seed-preferred promoters include, but are not limited to, Ciml (cytokinin-induced message), cZ19Bl (maize 19 kDa zein), milps (myo-inositol-1-phosphate synthase), and celA (cellulose synthase) (see WO 00/11177, herein incorporated by reference). Gama-zein is a preferred endosperm-specific promoter. Glob-1 is a preferred embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, globulin 1, etc. See also WO 00/12733, where seed-preferred promoters from end1 and end2 genes are disclosed; herein incorporated by reference.

In certain embodiments, the polynucleotides of the present disclosure can involve the use of the intact, native leghemoglobin genes, wherein the expression is driven by a cognate 5′ upstream promoter sequence(s).

In certain embodiments of the compositions and methods described herein, the promoter is a leaf-preferred promoter.

In certain embodiments of the compositions and methods described herein, the leghemoglobin coding sequence encodes a polypeptide having at least or at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2, and variants thereof. As used herein “variant” refers to a protein or polypeptide derived from a native protein or polypeptide by deletion or addition of one or more amino acids at one or more internal sites in the native protein or polypeptide and/or substitution of one or more amino acids at one or more sites in a native protein or polypeptide. Variants encompassed by the present disclosure exhibit a biological activity of the native protein or polypeptide sequence. Biologically active variants of a native protein of the embodiments can have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequence for the native protein as determined by sequence alignment programs known in the art. A biologically active variant of a protein of the present disclosure can differ from that protein by as few as about 1-15 amino acid residues, as few as about 1-10, such as about 6-10, as few as about 5, as few as 4, 3, 2, or even 1 amino acid residue.

As used herein “encoding,” “encoded,” or the like, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference).

As used herein “percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

Unless otherwise stated, sequence identity/similarity values provided herein refer to the value obtained using the BLAST 2.0 suite of programs using default parameters (Altschul, et al., (1997) Nucleic Acids Res. 25:3389-402).

Provided are polynucleotide and polypeptide sequences which have at least or at least about 50%, 55%, 60%, 65%, 70%, 75%, 80%, 85%, 86%, 87%, 88%, 89%, 90%, 91%, 92%, 93%, 94%, 95%, 96%, 97%, 98%, 99%, 99.5%, 99.6%, 99.7%, 99.8% or 99.9% and less than 100%, 99%, 95% or 90% identity to the polypeptides and polynucleotides of any one of SEQ ID NOs: 1-52, or to specified sequences within defined positions of any one of SEQ ID NOs: 1-52, such as disclosed herein.

In certain embodiments, the plant leaf genome has been modified to introduce an insertion, deletion, and/or substitution into a native leghemoglobin gene. In certain embodiments, the plant leaf genome has been modified to introduce a heterologous regulatory enhancer or heterologous promoter sequence operably linked to the native leghemoglobin coding sequence. In certain embodiments, the promoter sequence comprises a polynucleotide having at least 50%, 55%, 60%, 65%, 70%, 75%, 80%, 85%, 86%, 87%, 88%, 89%, 90%, 91%, 92%, 93%, 94%, 95%, 96%, 97%, 98%, 99%, 99.5%, 99.6%, 99.7%, 99.8% or 99.9% and less than 100%, 99%, 95% or 90% identity to any one of SEQ ID NOs: 8-9. In certain embodiments, the plant leaf genome has been modified to introduce a substitution that creates or enhances a native leghemoglobin coding sequence regulatory enhancer or a native leghemoglobin coding sequence promoter sequence.

The genomic sequence of the soybean leghemoglobin gene is provided in SEQ ID NO: 3 and modifications may be made to or include all or part of this sequence or to a homologous sequence corresponding to SEQ ID NO: 3 in the plant genome, including to specific regions identified herein. With respect to SEQ ID NO: 3, the regulatory region, including the promotor and 5′ UTR, is from nucleotide position 1 to position 2058, exon 1 is from position 2059 to position 2156, intron 1 is from position 2157 to position 2275, exon 2 is from position 2276 to position 2384, intron 2 is from position 2385 to position 2574, exon 3 is from position 2575 to position 2679, intron 3 is from position 2680 to position 2876, exon 4 is from position 2877 to position 3002, the terminator, including the 3′ UTR, is from position 3003 to position 5214.

In some embodiments, the modification is made from position 1-2058 of SEQ ID NO: 3, 100-2058 of SEQ ID NO: 3, 200-2058 of SEQ ID NO: 3, 300-2058 of SEQ ID NO: 3, 400-2058 of SEQ ID NO: 3, 500-2058 of SEQ ID NO: 3, 600-2058 of SEQ ID NO: 3, 700-2058 of SEQ ID NO: 3, 800-2058 of SEQ ID NO: 3, 900-2058 of SEQ ID NO: 3, 1000-2058 of SEQ ID NO: 3, 1100-2058 of SEQ ID NO: 3, 1200-2058 of SEQ ID NO: 3, 1300-2058 of SEQ ID NO: 3, 1400-2058 of SEQ ID NO: 3, 1500-2058 of SEQ ID NO: 3, 1600-2058 of SEQ ID NO: 3, 1700-2058 of SEQ ID NO: 3, 1800-2058 of SEQ ID NO: 3, or 1900-2058 of SEQ ID NO: 3.

As used herein an “amino acid deletion,” “deletion mutation,” or the like, refers to a mutation in which the indicated amino acid residue is removed from the polypeptide sequence, so that, when aligned to a reference sequence (e.g., SEQ ID NO: 2) the modified sequence does not have an amino acid corresponding to the indicated position of the reference sequence. An “amino acid insertion,” “insertion mutation,” or the like, refers to a mutation in which at least one amino acid residue is added to the polypeptide sequence, so that, when aligned to the reference a sequence (e.g., SEQ ID NO: 2) the modified sequence contains an additional amino acid corresponding to the indicated position of the reference sequence. An “amino acid substitution,” “substitution mutation,” or the like, refers to a mutation in which the indicated amino acid residue is replaced with a different amino acid residue, so that, when aligned to the reference sequence (e.g., SEQ ID NO: 2) the modified sequence does not have the same amino acid at the indicated position.

In certain embodiments, the plant leaf genome has been modified to replace all or part of a coding sequence of a non-leghemoglobin gene with a leghemoglobin coding sequence. Such an edited construct comprising an exogenous nucleic acid coding sequence operably linked to a native promoter in its native position in the genome would not be considered a recombinant construct, because the promoter and other regulatory elements are not exogenous to their native environment. For example, in an edited genome, the gene structure can remain largely unaltered, with the native seed-storage protein coding sequence being replaced by a different coding sequence, such as with a globulin protein, such as leghemoglobin. Such plants, seeds and cells may be referred to as modified or edited plants, seeds or cells. The non-leghemoglobin gene is not particularly limited and may be any gene which allows for expression of the introduced leghemoglobin coding sequence. In certain embodiments, the non-leghemoglobin gene is a gene that is highly and/or preferentially expressed in tissue such as leaves. In certain embodiments, the non-leghemoglobin gene is a ribulose-1,5-bisphosphate carboxylase-oxygenase (RUBISCO) gene (e.g., Glyma. 13g046200, Glyma. 19g046800), a RUBISCO Activase (RCA) gene (e.g., Glyma.11g221000, Glyma.02g249600, Glyma.14g067000), or a vegetative storage protein (VSP) gene (e.g., Glyma.07g014500, Glyma.08g200100, Glyma.08g200200, Glyma.15g218400, Glyma.08g200000).

In certain embodiments, the plant tissue such as leaves can increase expression of leghemoglobin which forms a heme complex without the need to target expression of the leghemoglobin to a protein storage vesicle or other targeted cellular compartment. In certain embodiments, the leghemoglobin coding sequence of the recombinant construct, the modified native leghemoglobin gene or coding sequence, and/or the inserted leghemoglobin coding sequence further comprises an operably linked targeting sequence. The operably linked targeting sequence can be part of the recombinant construct or can be introduced by genome modification. In certain embodiments, the targeting sequence targets the leghemoglobin to an intracellular compartment. In certain embodiments, the intracellular compartment is a plastid. In certain embodiments, the targeting sequence comprises a polynucleotide encoding a polypeptide having at least 65%, 70%, 75% 80%, 85%, 90%, 95%, or 99% identity to SEQ ID NO: 53. In certain embodiments, the intracellular compartment is the endoplasmic reticulum (ER). In certain embodiments, the targeting sequence comprises a polynucleotide encoding a polypeptide having at least 65%, 70%, 75% 80%, 85%, 90%, 95%, or 99% identity to SEQ ID NO: 54.

In certain embodiments, the targeting sequence, also referred to herein as a transit sequence, such as a plastid targeting sequence, is included and operably linked to a sequence encoding leghemoglobin, such as being placed just before the N′ terminus of a sequence encoding leghemoglobin, such that the targeting sequence targets expression of the leghemoglobin to an intracellular compartment such as the endoplasmic reticulum (ER) or a plastid. The targeting sequence and operably linked leghemoglobin sequence, such as occurs in SEQ ID NO: 4 or 6 or a polynucleotide encoding SEQ ID NO: 5 or 7, can be operably linked to a regulatory sequence in a recombinant construct and used to transform a plant. The targeting sequence can be operably linked to a leghemoglobin sequence, such as occurs in SEQ ID NO: 4 or 6, or a sequence encoding SEQ ID NO: 5 or 7, and can be inserted through genome editing to replace all or part of the coding sequence of a non-leghemoglobin protein such as RUBISCO, RCA, or VSP, such that the native regulatory elements of the non-leghemoglobin protein direct expression of the targeting sequence and the leghemoglobin coding sequence such that the leghemoglobin protein is expressed with a transit peptide and targeted to an intracellular compartment. The targeting sequence can be inserted into the native leghemoglobin gene, optionally with other insertions, or deletions or substitutions, so that leghemoglobin is expressed in the plant leaf from its native locus with a transit peptide and targeted to an intracellular compartment. In one embodiment the plastid targeting sequence is included at the N′ terminus of the coding sequence or polypeptide of interest. One example of a plastid targeting sequence is the Rubisco SSUSP plastid targeting sequence, such as encoded by the nucleotide sequence from position 1 to position 165 of SEQ ID NO: 4, with the corresponding peptide targeting sequence at position 1 to position 55 of SEQ ID NO: 5. The leghemoglobin coding sequence is from position 166 to position 603 of SEQ ID NO: 4 and the corresponding peptide form position 56 to position 200 of SEQ ID NO: 5.

In some embodiments, plant tissue, plant leaves and plants are provided which express leghemoglobin from two or more sources, constructs or genomic locations, such as from two or more of (i) a recombinant construct inserted into the genome, (ii) a genome modification in which the leghemoglobin coding sequence replaces all or part of a non-leghemoglobin coding sequence such as described herein (iii) a genome modification in which the native leghemoglobin gene is modified to include one or more of an insertion, deletion or substitution, such as into the regulatory region or coding sequence of the leghemoglobin gene and (iv) a plastid genome modification in which the plastid genome is modified to express a leghemoglobin coding sequence. In some embodiments, the two or more sources include at least one source in which the leghemoglobin coding sequence is operably linked to an intracellular targeting sequence, such as a plastid or endoplasmic reticulum targeting sequence as described herein, and another source in which the leghemoglobin coding sequence is not operably linked to an intracellular targeting sequence.

In certain embodiments, the plant tissue such as leaves and/or plants of the compositions and methods described herein further comprise a modification to increase the amount of leghemoglobin complex in the leaf. In certain embodiments, the modification to increase the amount of leghemoglobin complex comprises increasing expression of one or more (e.g., 2, 3, 4, 5, 6, 7, 8, 9, or 10 or more) porphyrin pathway genes. In certain embodiments, the porphyrin pathway gene is operably linked to a heterologous regulatory element. The modification can include the introduction of a recombinant construct into the genome of the plant, or the modification can include a gene editing modification, such as an insertion, deletion and/or substitution into the genes from which these polypeptides are expressed, such as to enhance transcription of the coding sequences of these genes. In certain embodiments, the porphyrin pathway gene is introduced into the leaf in a recombinant construct. In certain embodiments, the genome of the plant leaf is modified to increase expression of the porphyrin pathway gene. For example, in certain embodiments the plant leaf is modified to have one or more nucleotide insertions, substitutions, and/or deletions to generate a plant leaf comprising the one or more porphyrin pathway genes operably linked to a heterologous regulatory element. Porphyrin pathway genes for use in the compositions and methods described herein include, but are not limited to, glutamyl-tRNA reductase, a ferrochelatase, a glutamate-1-semialdehyde 2, a 1-aminomutase, an aminolevulinate dehydratase, a hydroxymethylbilane synthase, a urophorphyrinogen III synthase, a urophorphyrinogen decarboxylase, a coporphyrinogen III oxidase, and a protoporphyrinogen oxidase, or any combination thereof.

In some embodiments, the plant tissue such as leaves and/or plants comprising the plant leaves comprise modifications in genes that encode regulatory proteins that modulate expression or activity of enzymes contributing to heme production or hemelation of leghemoglobin. For example, genes encoding proteins that regulate glutamyl-tRNA reductase activity include, for example, glutamyl-tRNA reductase-binding protein (Glyma.08G222600), chloroplast signal particle 43 (Glyma.11G097200) and FLUORESCENT IN BLUE LIGHT (Glyma. 16G010200 and Glyma.07G041700) can be modified, such as by insertion, deletion or substitution to increase or enhance the formation of heme and/or the leghemoglobin complex in the plant leaf. Alternatively, the genes that encode regulatory proteins that modulate expression or activity of enzymes contributing to heme production or hemelation of leghemoglobin may be introduced into the plant and/or plant leaf using a recombinant construct comprising the gene.

In some embodiments, the plant tissue such as leaves and/or plants described herein containing leghemoglobin protein in an amount of at least 0.1% of total protein have a genomic modification which includes at least one of (i) a nucleic acid insertion of a genomic sequence, (ii) one or more nucleic acid substitutions, (iii) one or more nucleic acid deletions, and (iv) any combination thereof, wherein the genomic modification comprises (a) a modification made to the native leghemoglobin gene or (b) an insertion comprising at least a portion of the native leghemoglobin gene.

In certain embodiments, the plants and/or plant tissue such as leaves of the compositions and methods described herein further comprise at least one addition modification that increases total protein content in the leaf as compared to a control plant leaf (e.g., leaf not comprising the at least one modification). In certain embodiments, the plant leaf comprising the at least one modification comprises at least about a 1%, 1.5%, 2%, 2.5%, 3%, 3.5%, 4%, 5%, 10%, or 15% and less than 20%, 15%, 10%, 9%, 8%, 7%, 6%, or 5% percentage point increase in total protein measured on a dry weight basis, as compared to a control plant leaf. As used herein, “percent increase” refers to a change or difference expressed as a fraction of the control value, e.g. {[modified/transgenic/test value (%)−control value (%)]/control value (%)}×100%=percent change, or {[value obtained in a first location (%)−value obtained in second location (%)]/value in the second location (%)}×100=percent change. The modification may be introduced by a recombinant construct or by genome modification. Non-limiting examples of modifications include a modification of one or more of a gene encoding (i) a CCT-domain containing protein, (ii) a reticulon, (iii) a trehalose phosphate synthase, (iv) a HECT Ubiquitin Ligase (HEL or UPL3), (v) a MFT (mother of flowering) polypeptide, (vi) a raffinose synthase RS2, RS3, or RS4, such as disclosed in U.S. Pat. Nos. 5,710,365, 8,728,726, and 10,081,814 each of which are incorporated herein by reference in their entirety or (vii) any combination thereof.

Also provided herein are plants comprising any of the plant tissue such as leaves described herein. Such that in certain embodiments, provided are plants comprising a leghemoglobin protein in a leaf in an amount of at least 0.01%, 0.05%, 0.5%, 1%, 1.5%, 2%, 2.5%, 3%, 4%, 5%, 6%, 7%, 8%, 9%, 10% or more and less than 75%, 50%, 25%, 20%, 15%, 10%, 5%, 4%, or 3% of the total leaf protein. The plant may comprise any modification described herein to express the leghemoglobin in the leaf, such as, for example, the plant may comprise the leghemoglobin coding sequence introduced by genetic modification or by a recombinant construct. The plant may further comprise any additional modification described herein, such as, for example, a modification of porphyrin pathway gene and/or a modification to increase protein content. In certain embodiments, the plant modification or recombinant construct introduction results in the coding sequence being preferentially expressed in the leaf of the plant, or the modification or introduction may result in increased expression of the coding sequence in multiple tissues of the plant or in the total plant, or a combination thereof.

In certain embodiments, the plants described herein comprising plant tissue such as leaves described herein are elite plant lines (e.g., elite soybean line, elite pea line, elite alfalfa line, elite maize line). In certain embodiments, the plant cells, plant parts, seeds, and grain are isolated from or produced by an elite plant line. As used herein, “elite line” refers to any line that has resulted from breeding and selection for superior agronomic performance that allows a producer to harvest a product of commercial significance. Numerous elite lines are available and known to those of skill in the art of plant breeding (e.g., soybean, pea, canola, maize, alfalfa, wheat and sunflower breeding). An “elite population” is an assortment of elite individuals or lines that can be used to represent the state of the art in terms of agronomically superior genotypes of a given crop species, such as soybean. As used herein, the term “plant” includes plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, cars, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the disclosure, provided that these parts comprise the introduced polynucleotides.

Tissue of plants which can be modified according to the methods disclosed herein includes seeds, fruits and flowers as well as vegetative tissue (e.g., above-ground vegetative tissue and below-ground vegetative tissue). Vegetative tissue which can be modified according to the methods described herein includes non-seed plant parts such as roots, shoot buds, stems and leaves, including above-ground and below ground vegetative tissue. Above-ground vegetative tissue which can be modified according to the methods described herein includes stems, leaves, shoots and other vegetative above-ground tissues and excludes seeds and organs typically found underground for that plant such as roots, bulbs, tubers, corms, caudices, underground stems and rhizomes. Below-ground vegetative tissue which can be modified according to the methods described herein includes roots, bulbs, tubers, corms, caudices, underground stems and rhizomes, including for example potatoes, carrots, yams, beets, parsnips, turnips, rutabagas, yuca, kohlrabi, onions, shallots, garlic, celeriac, horseradish, daikon, turmeric, jicama, Jerusalem artichokes, radishes, and ginger and excludes organs typically found above ground for that plant. Seeds, fruits and flowers which can be modified according to the methods described herein include without limitation maize kernels, soybean seeds, alfalfa seeds, pea seeds, canola seeds, sunflower seeds, wheat seeds, barley seeds, rye seeds, oat seeds, tomatoes, bananas, grapes, apples, pears, durian, lychee, melons such as watermelon and cantaloupe, oranges, lemons, limes and citrus fruits, strawberries, blackberries, blueberries, raspberries and berry fruits, peaches, plums, nectarines, cherries, apricots, mango, avocado, pineapple, squash such as cucumber, pumpkin, zucchini, coconut, papaya, dragon fruit, fig, gooseberry, guava, jackfruit, kumquat, kiwifruit, persimmon, starfruit, allium, nasturtium, marigold, pansy, calendula, hibiscus, and roses.

The plant species and plant tissue such as leaves of the compositions and methods of the present disclosure can be any plant species or plant tissue for which expression of a leghemoglobin polypeptide described herein is desired, including, but not limited to, monocots and dicots. Examples of plants and plant tissue such as leaves of interest include, but are not limited to those of corn (Zea mays), Brassica spp. (e.g., Brassica napus, Brassica rapa, Brassica juncea), particularly those Brassica species useful as sources of seed oil, alfalfa (Medicago sativa), rice (Oryza sativa), rye (Secale cereale), pea, including (Pisum sativum), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatas), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Per Sea americana), fig (Ficus casica), guava (Psidium guajava), mango (Mangifera indica), olive (Olea europaea), papaya(Carica papaya), cashew (Anacardium occidentale), macadamia (Macadamia integrifolia), almond (Prunus amygdalus), sugar beets (Beta vulgaris), sugarcane (Saccharum spp.), oats (Avena sativa), barley (Hordeum vulgare), vegetables, ornamentals, and conifers.

In certain embodiments, the plant and/or plant leaf of the compositions and methods described herein is a legume crop species, including, but not limited to, alfalfa (Medicago sativa); clover or trefoil (Trifolium spp.); pea, including (Pisum sativum), pigeon pea (Cajanus cajan), cowpea (Vigna unguiculata) and Lathyrus spp.; bean (Fabaceae or Leguminosae); lentil (Lens culinaris); lupin (Lupinus spp.); mesquite (Prosopis spp.); carob (Ceratonia siliqua), soybean (Glycine max), peanut (Arachis hypogaea) or tamarind (Tamarindus indica).

In certain embodiments, the plant and/or plant leaf of the compositions and methods described herein is selected from the group consisting of soybean, pea, alfalfa, sunflower, maize, sorghum, rice, and brassica.

In certain embodiments, the plants described herein, e.g., plants comprising a leghemoglobin protein in a leaf or plant tissue (such as vegetative tissue), such as in an amount of at least 0.01%, 0.05%, 0.5%, 1%, 1.5%, 2%, 2.5%, 3%, 4%, 5%, 6%, 7%, 8%, 9%, 10% or more and less than 75%, 50%, 25%, 20%, 15%, 10%, 5%, 4%, or 3% of the total leaf protein or total tissue protein, further comprise a seed leghemoglobin, such as in amount of at least 0.01%, 0.05%, 0.5%, 1%, 1.5%, 2%, 2.5%, 3%, 4%, 5%, 6%, 7%, 8%, 9%, 10% or more and less than 75%, 50%, 25%, 20%, 15%, 10%, 5%, 4%, or 3% of the total seed protein. In some embodiments, the plants and seeds described herein may express leghemoglobin in both the seeds and leaves, and in some embodiments in other plant tissues such as stems and roots with modifications introducing through any of the techniques described herein, including without limitation genome editing and transformation with recombinant constructs, or any combination thereof. Accordingly, provided are plants comprising a leghemoglobin protein in a leaf in an amount of at least 0.01%, 0.05%, 0.5%, 1%, 1.5%, 2%, 2.5%, 3%, 4%, 5%, 6%, 7%, 8%, 9%, 10% or more and less than 75%, 50%, 25%, 20%, 15%, 10%, 5%, 4%, or 3% of the total leaf protein and a leghemoglobin protein in a seed of at least 0.01%, 0.05%, 0.5%, 1%, 1.5%, 2%, 2.5%, 3%, 4%, 5%, 6%, 7%, 8%, 9%, 10% or more and less than 75%, 50%, 25%, 20%, 15%, 10%, 5%, 4%, or 3% of the total seed protein. The leghemoglobin in the seed and leaf can be a leghemoglobin without a heme group, the leghemoglobin complex, or a combination of both forms. The modifications to increase seed, leaf, and/or plant tissue leghemoglobin may be introduced using recombinant constructs or gene editing in any combination. Modifications to increase soybean seed leghemoglobin content are known in the art, such as, for example, the modifications described in U.S. Pat. No. 11,359,206, which is incorporated herein in its entirety by reference.

In certain embodiments, the plants described herein, e.g., plants comprising a leghemoglobin protein in a leaf or above-ground vegetative tissue, such as in an amount of at least 0.01%, 0.05%, 0.5%, 1%, 1.5%, 2%, 2.5%, 3%, 4%, 5%, 6%, 7%, 8%, 9%, 10% or more and less than 75%, 50%, 25%, 20%, 15%, 10%, 5%, 4%, or 3% of the total leaf protein or total above-ground vegetative tissue protein, have been further modified to increase expression of a root or below-ground tissue leghemoglobin, such as in amount of at least 0.01%, 0.05%, 0.5%, 1%, 1.5%, 2%, 2.5%, 3%, 4%, 5%, 6%, 7%, 8%, 9%, 10% or more and less than 75%, 50%, 25%, 20%, 15%, 10%, 5%, 4%, or 3% of the total root protein or total below-ground tissue protein. The modifications to increase root or below-ground tissue leghemoglobin may be introduced using recombinant constructs or gene editing.

In some embodiments, plants are grown hydroponically in non-soil media, such as water, rockwool, perlite, vermiculite, sand, gravel, baked clay, sawdust, sphagnum peat moss, oasis rice hulls, polyurethane, or coconut fiber. Methods of harvesting or extracting leghemoglobin from whole plants including root or below-ground tissues or from root or below-ground tissues include the steps of growing plants hydroponically and processing the plants or below-ground tissues to extract the leghemoglobin.

In certain embodiments, the plants described herein comprise a below-ground tissue or root expressed leghemoglobin in combination with a leaf or above-ground tissue leghemoglobin in combination with a seed expressed leghemoglobin. The modifications to increase leghemoglobin into the plant may be introduced using recombinant constructs or gene editing, such as by directly modifying a plant cell at a different location which has been previously modified to express a below-ground, above-ground or seed leghemoglobin. The modifications to increase leghemoglobin into the plant may be introduced or combined by introgression in which a first plant comprising one or more of the modifications provided herein is crossed with a second plant comprising one or more modifications, at least one of which is different from the modifications comprised in the first plant and a plant or seed comprising at least one modification from the first plant and the different modification of the second plant is selected following the cross. Selfing, further crossing and backcrossing may be used if desired to adjust the genetics of the resulting plant. The total leghemoglobin protein in the plant may be in an amount of at least 0.01%, 0.05%, 0.5%, 1%, 1.5%, 2%, 2.5%, 3%, 4%, 5%, 6%, 7%, 8%, 9%, 10% or more and less than 75%, 50%, 25%, 20%, 15%, 10%, 5%, 4%, or 3% of the total plant protein.

In certain embodiments, the plants described herein, e.g., plants comprising a leghemoglobin protein in a leaf or plant tissue, or in any or all combinations of leaf tissue, plant tissue and seeds provide a yield of leghemoglobin per acre of at least or at least about 0.01 kg, 0.02 kg, 0.03 kg, 0.04 kg, 0.05 kg, 0.06 kg, 0.07 kg, 0.08 kg, 0.09 kg, 0.1 kg, 0.2 kg, 0.3 kg, 0.4 kg, 0.5 kg, 1 kg, 1.5 kg, 2 kg, 2.5 kg, 3 kg, 4 kg, 5 kg, 6 kg, 7 kg, 8 kg, 9 kg, 10 kg, 15 kg, 20 kg or 25 kg and less than or less than about 500 kg, 400 kg, 300 kg, 250 kg, 200 kg, 150 kg, 100 kg, 90 kg, 80 kg, 70 kg, 60 kg, 55 kg, 50 kg, 45 kg, 40 kg, 35 kg or 30 kg. For example, provided are peas, alfalfa, and/or soybean plants comprising a leghemoglobin protein in a leaf, and optionally in a seed, comprising a yield of leghemoglobin per acre of at least or at least about 0.01 kg, 0.02 kg, 0.03 kg, 0.04 kg, 0.05 kg, 0.06 kg, 0.07 kg, 0.08 kg, 0.09 kg, 0.1 kg, 0.2 kg, 0.3 kg, 0.4 kg, 0.5 kg, 1 kg, 1.5 kg, 2 kg, 2.5 kg, 3 kg, 4 kg, 5 kg, 6 kg, 7 kg, 8 kg, 9 kg, 10 kg, 15 kg, 20 kg or 25 kg and less than or less than about 500 kg, 400 kg, 300 kg, 250 kg, 200 kg, 150 kg, 100 kg, 90 kg, 80 kg, 70 kg, 60 kg, 55 kg, 50 kg, 45 kg, 40 kg, 35 kg or 30 kg.

Methods of cultivating and harvesting plants to provide a yield of leghemoglobin per area are provided. In some embodiments, the yield of leghemoglobin per acre may be at least or at least about 0.01 kg, 0.02 kg, 0.03 kg, 0.04 kg, 0.05 kg, 0.06 kg, 0.07 kg, 0.08 kg, 0.09 kg, 0.1 kg, 0.2 kg, 0.3 kg, 0.4 kg, 0.5 kg, 1 kg, 1.5 kg, 2 kg, 2.5 kg, 3 kg, 4 kg, 5 kg, 6 kg, 7 kg, 8 kg, 9 kg, 10 kg, 15 kg, 20 kg or 25 kg and less than or less than about 500 kg, 400 kg, 300 kg, 250 kg, 200 kg, 150 kg, 100 kg, 90 kg, 80 kg, 70 kg, 60 kg, 55 kg, 50 kg, 45 kg, 40 kg, 35 kg or 30 kg. The methods include one or more steps of planting plants modified as disclosed herein, growing the plants, harvesting plant tissue or the entire plant and extracting leghemoglobin from the plant or plant tissue.

In certain embodiments, the plant (comprising for example vegetative tissue, leaf, fruit, tuber, stem, root) expressing leghemoglobin as described herein further comprises a modification to reduce expression of storage or other proteins to help drive expression of protein towards leghemoglobin. The modification to reduce or knock out storage or other proteins can be done though genome-editing, transformation, or mutation or native trait breeding and may be combined with the modification expressing leghemoglobin by either genetic crosses, or by performing gene editing or transformation in the leghemoglobin over-expression plants, or by expressing the leghemoglobin cassettes into plants that contain the reduced or knocked out storage or other protein. With a reduction of storage or other proteins in plants, the content of leghemoglobin increases to compensate for the loss of storage or other protein through protein rebalancing.

In certain embodiments, the plant (comprising for example vegetative tissue, leaf, fruit, tuber, stem, root) expressing leghemoglobin as described herein further comprises a modification to reduce expression of storage carbohydrate or fats to help drive carbon towards expression of leghemoglobin. The modification to reduce or knock out expression of storage fats or carbohydrates can be done though genome-editing, transformation, or mutation or native trait breeding and may be combined with the modification expressing leghemoglobin by either genetic crosses, or by performing gene editing or transformation in the leghemoglobin over-expression plants, or by expressing the leghemoglobin cassettes into plants that contain the reduced or knocked out storage fat or carbohydrate. With a reduction in storage fats or carbohydrates in plants, the content of leghemoglobin increases to compensate the loss of storage fat or carbohydrate through carbon rebalancing.

In some embodiments, the plant or plant tissue is further modified to alter the fatty acid content in the plant or plant tissue, such as to increase oleic acid, reduce linolenic acid, or a combination thereof. The oleic acid may be increased by at least about 1%, 2%, 3%, 5%, 10%, 20%, 30%, 40%, 50%, 60%, 70%, 80% or 90% and less than about 95%, 90%, 80%, 70%, 60%, 50% 40%, 30% or 20%. The linoleic acid may be reduced by at least about 1%, 2%, 3%, 5%, 10%, 20%, 30%, 40%, 50%, 60%, 70%, 80% or 90% and less than about 95%, 90%, 80%, 70%, 60%, 50% 40%, 30% or 20%.

Also provided are methods for producing the plants and plant tissue such as leaves described herein. The methods comprising introducing the sequences described herein (e.g., leghemoglobin coding sequence, porphyrin pathway gene, and/or gene to increase protein content) using gene editing, recombinant constructs, or a combination thereof. The sequences may be introduced directly into leaf tissue. Alternatively, the sequences may be introduced into regenerable plant cell which can be grown into a plant comprising tissue such as leaves expressing a leghemoglobin protein.

The genome editing technology for use in the methods and compositions described herein is not particularly limited and may be any genome editing technique that allows for the introduction and/or targeted introduction of the desired polynucleotide.

In certain embodiments, the method comprises: (a) providing a guide RNA, at least one polynucleotide modification template, and at least one Cas endonuclease to a plant cell, wherein the at least one Cas endonuclease introduces a double stranded break at an endogenous gene to be modified in the plant cell, and wherein the polynucleotide modification template generates a modified gene that encodes any of the polypeptides described herein; (b) obtaining a plant from the plant cell; and (c) generating a progeny plant.

In certain embodiments the genome editing technique is selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, and any combination thereof.

In certain embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, and the like. In some embodiments, the introduction of a DSB can be combined with the introduction of a polynucleotide modification template.

The process for editing a genomic sequence combining DSB and modification templates generally comprises providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

TAL effector nucleases (TALEN) are a class of sequence-specific nucleases that can be used to make double-strand breaks at specific target sequences in the genome of a plant or other organism (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H—N—H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18-nucleotide recognition sequence.

Genome editing using DSB-inducing agents, such as Cas9-gRNA complexes, has been described, for example in U.S. Patent Application US 2015-0082478 A1, WO2015/026886 A1, WO2016007347, and WO201625131 all of which are incorporated by reference herein.

In certain embodiments the genetic modification is introduced without introducing a double strand break using base editing technology, see e.g., Gaudelli et al., (2017) Programmable base editing of A*T to G*C in genomic DNA without DNA cleavage. Nature 551(7681): 464-471; Komor et al., (2016) Programmable editing of a target base in genomic DNA without double-stranded DNA cleavage, Nature 533(7603): 420-4.

In certain embodiments, base editing comprises (i) a catalytically impaired CRISPR-Cas9 mutant that is mutated such that one of their nuclease domains cannot make DSBs; (ii) a single-strand-specific cytidine/adenine deaminase that converts C to U or A to G within an appropriate nucleotide window in the single-stranded DNA bubble created by Cas9; (iii) a uracil glycosylase inhibitor (UGI) that impedes uracil excision and downstream processes that decrease base editing efficiency and product purity; or (iv) nickase activity to cleave the non-edited DNA strand, followed by cellular DNA repair processes to replace the G-containing DNA strand.

Similarly, the method for introducing the recombinant constructs in not particularly limited and may be any method that allows for the recombinant construct to express the coding sequence in the leaf tissue.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606), Agrobacterium-mediated transformation (U.S. Pat. Nos. 5,563,055 and 5,981,840), Ochrobacterium-mediated transformation (U.S. Patent Application Publication 2018/0216123 and WO20/092494) direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

“Stable transformation” is intended to mean that the polynucleotide introduced into a plant integrates into the genome of the plant of interest and is capable of being inherited by the progeny thereof. “Transient transformation” is intended to mean that a polynucleotide is introduced into the plant of interest and does not integrate into the genome of the plant or organism, or a polypeptide is introduced into a plant or organism.

In specific embodiments, the sequences described herein can be provided to a plant using a variety of transient transformation methods. Such transient transformation methods include, but are not limited to, the introduction of the leghemoglobin protein directly into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91:2176-2180 and Hush et al. (1994) The Journal of Cell Science 107:775-784, all of which are herein incorporated by reference.

In other embodiments, the inventive polynucleotides disclosed herein may be introduced into plants by contacting plants with a virus or viral nucleic acids. Generally, such methods involve incorporating a nucleotide construct of the disclosure within a DNA or RNA molecule. It is recognized that the inventive polynucleotide sequence may be initially synthesized as part of a viral polyprotein, which later may be processed by proteolysis in vivo or in vitro to produce the desired recombinant protein. Further, it is recognized that promoters disclosed herein also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide disclosed herein can be contained in a transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided, and the transfer cassette is integrated at the target site. The polynucleotide of interest is thereby integrated at a specific chromosomal position in the plant genome. Other methods to target polynucleotides are set forth in WO 2009/114321 (herein incorporated by reference), which describes “custom” meganucleases produced to modify plant genomes, in particular the genome of maize. Sec, also, Gao et al. (2010) Plant Journal 1:176-187.

One of skill will recognize that after the expression cassette containing the inventive polynucleotide is stably incorporated in transgenic plants and confirmed to be operable, it can be introduced into other plants by sexual crossing. Any of a number of standard breeding techniques can be used, depending upon the species to be crossed.

Further provided are methods of plant breeding comprising crossing any of the plants described herein (e.g., soybean, pea, alfalfa) with a second plant to produce a progeny seed comprising at least one modification described herein. In certain embodiments, a plant is produced from the progeny seed. In certain embodiments, the method comprises crossing any of the plants described herein, e.g., plants comprising a leghemoglobin protein in a leaf in an amount of at least 0.01%, 0.05%, 0.5%, 1%, 1.5%, 2%, 2.5%, 3%, 4%, 5%, 6%, 7%, 8%, 9%, 10% or more and less than 75%, 50%, 25%, 20%, 15%, 10%, 5%, 4%, or 3% of the total leaf protein with a second plant comprising a seed leghemoglobin in amount of at least 0.01%, 0.05%, 0.5%, 1%, 1.5%, 2%, 2.5%, 3%, 4%, 5%, 6%, 7%, 8%, 9%, 10% or more and less than 75%, 50%, 25%, 20%, 15%, 10%, 5%, 4%, or 3% of the total seed protein to produce a progeny seed, and generating a plant wherein the plant comprises a leghemoglobin protein in the leaf in an amount of at least 0.01%, 0.05%, 0.5%, 1%, 1.5%, 2%, 2.5%, 3%, 4%, 5%, 6%, 7%, 8%, 9%, 10% or more and less than 75%, 50%, 25%, 20%, 15%, 10%, 5%, 4%, or 3% of the total leaf protein and a leghemoglobin protein in the seed in an amount of at least 0.01%, 0.05%, 0.5%, 1%, 1.5%, 2%, 2.5%, 3%, 4%, 5%, 6%, 7%, 8%, 9%, 10% or more and less than 75%, 50%, 25%, 20%, 15%, 10%, 5%, 4%, or 3% of the total seed protein.

In some embodiments, methods for processing leghemoglobin compositions extracted from the plant tissue such as leaves which express leghemoglobin are provided in which the leghemoglobin composition is contacted with at least one of a cellulase, a hemicellulase, and a pectinase under conditions sufficient to degrade the polysaccharides in the leghemoglobin composition and the permeant is filtered from the residue. leghemoglobin composition extracted from the plant tissue such as leaves is provided containing at least 0.1%, 0.2%, 0.3%, 0.4% or 0.5% leghemoglobin by weight (wt) total protein.

In some embodiments, an isolate is provided which comprises at least 0.1%, 0.2%, 0.3%, 0.4%, 0.5%, 1%, 2%, 3%, 4%, 5%, 6%, 7%, 8%, 9% or 10% and less than 25%, 20%, 15%, 10%, 9%, 8%, 7%, 6%, 5%, 4%, 3%, 2% or 1% leghemoglobin by weight of total protein, wherein at least about 25%, 30%, 35%, 40%, 45%, 50%, 55%, 60%, 65%, 70%, 80%, 85%, 90% or 95% and less than 99.9%, 99%, 98%, 97%, 96%, 95%, 90%, 85%, 80%, or 75% of the leghemoglobin is hemelated with an iron group.

In some embodiments, plants, leaves, roots, seeds, fruits, flowers, and vegetative tissue are provided which comprise at least 0.01%, 0.05%, 0.1%, 0.2%, 0.3%, 0.4%, 0.5%, 1%, 2%, 3%, 4%, 5%, 6%, 7%, 8%, 9% or 10% and less than 25%, 20%, 15%, 10%, 9%, 8%, 7%, 6%, 5%, 4%, 3%, 2% or 1% leghemoglobin by weight of total protein, wherein at least about 5%, 10%, 15%, 20%, 25%, 30%, 35%, 40%, 45%, 50%, 55%, 60%, 65%, 70%, 80%, 85%, 90% or 95% and less than 99.9%, 99%, 98%, 97%, 96%, 95%, 90%, 85%, 80%, 75%, 70%, 65%, 60%, 55% or 50% of the leghemoglobin is hemelated with an iron group.

The following are examples of specific embodiments of some aspects of the invention. The examples are offered for illustrative purposes only and are not intended to limit the scope of the invention in any way.

### Example 1

This example demonstrates the expression of soybean leghemoglobin protein in plant leaves.

A soybean leghemoglobin gene (Glyma.20g191200) was identified in the soybean genome. The gene (SEQ ID NO: 3) contains 4 exons, with its coding sequence (CDS) (SEQ ID NO: 1) encoding a leghemoglobin peptide (SEQ ID NO: 2). The soybean leghemoglobin sequence was expressed with no signal peptide, a chloroplast-targeting peptide or an endoplasmic reticulum (ER)-targeting peptide in plant leaves (Table 2). The Rubisco small subunit (Rubisco SSU) plastid targeting sequences were used to target the leghemoglobin protein to plastids. The Rubisco SSUSP plastid targeting sequence is encoded by the nucleotide sequence from position 1 to position 165 of SEQ ID NO: 4, with the corresponding peptide targeting sequence at position 1 to position 55 of SEQ ID NO: 5. The leghemoglobin coding sequence is from position 166 to position 600 of SEQ ID NO: 4 and the corresponding peptide form position 56 to position 200 of SEQ ID NO: 5. Four amino acids (KDEL) (SEQ ID NO: 54) were fused to the c-terminal of the leghemoglobin to provide endoplasmic reticulum (ER)-targeting, which is encoded by the nucleotide sequence from position 436 to position 447 of SEQ ID NO: 6, with the corresponding peptide targeting sequence at position 146 to position 149 of SEQ ID NO: 7. The leghemoglobin coding sequence is from position 1 to position 435 of SEQ ID NO: 6 and the corresponding peptide form position 1 to position 145 of SEQ ID NO: 7. Strong constitutive leaf-preferred promoter, such as a RUBISCO small subunit promoter (SEQ ID NO: 8) or a RUBISCO activase isoform 2 (RCA2) promoter (SEQ ID NO: 9) was used to drive the expression of the leghemoglobin in plant leaves. These expression vectors were introduced into plants, such as soybean and alfalfa, by Agrobacteria-mediated transformation. The results are described in Example 7. A similar technical approach can be used for expression of leghemoglobin in monocot leaves, such as maize and rice.

TABLE 2
Expression of Leghemoglobin by Protein Targeting in Plant Leaves

| Leghemoglobin (LH) with or without | Nucleotide | Peptide |
|---|---|---|
| Signal Peptide (Vector name) | SEQ ID NO: | SEQ ID NO: |
| No signal peptide::LH | SEQ ID NO: 1 | SEQ ID NO: 2 |
| RUBISCO SSUSP::LH | SEQ ID NO: 4 | SEQ ID NO: 5 |
| LH::KDEL | SEQ ID NO: 6 | SEQ ID NO: 7 |

### Example 2

This example demonstrates the improvement of soybean leghemoglobin expression level by porphyrin pathway engineering-glutamyl-tRNA reductase and ferrochelatase

To improve the plant leghemoglobin expression level in leaves, a porphyrin pathway engineering approach was employed. There are at least nine enzymatic steps for the porphyrin pathway leading to the heme biosynthesis. Among them, the glutamyl-tRNA reductase (glyma.04g089800) and ferrochelatase (glyma.04g050400) are tested for increasing heme production to facilitate higher leghemoglobin accumulation and heme loading in plant leaves. For this purpose, three additional soybean vectors are made, each of them contains the expression of glutamyl-tRNA reductase (SEQ ID NO: 10, 11) and ferrochelatase (SEQ ID NOs: 12, 13), in addition to the leghemoglobin expression cassettes in Example 1. The two biosynthetic genes are driven by strong constitutive promoters, such as Ubiquitin promoter (SEQ ID NO: 14) or EF1A promoter (SEQ ID NO: 15). In these three vectors, the expression cassettes of these two biosynthetic genes are stacked molecularly with the expression cassettes of the leghemoglobin with or without different signal peptide targeting sequences. These expression vectors are introduced into plants by Agrobacterial-mediated transformation. The results are described in Example 7.

### Example 3

This example demonstrates the improvement of leghemoglobin expression level by porphyrin enzyme modifications or expression.

A similar technical approach to the methods described in Example 2 is used to regulate other enzymatic steps for the porphyrin pathway, such as glutamate-1-semialdehyde 2,1-aminomutase, aminolevulinate dehydratase, hydroxymethylbilane synthase, urophorphyrinogen III synthase, urophorphyrinogen decarboxylase, coporphyrinogen III oxidase, and protoporphyrinogen oxidase. Examples of soybean genes for the porphyrin pathway that are used are listed in Table 3. Overexpressing these native metabolic enzyme genes in plant leaves is achieved by transformation of soybean with a recombinant construct comprising a coding sequence for these polypeptides, operably linked to regulatory sequences that provide for expression in plant leaves. Secondly, increased expression of these enzymes is achieved through gene editing. Feedback sensitive regulatory domains of these enzymes are identified and removed or inactivated by gene editing truncations, deletions, substitutions or insertions. It is expected that enhanced heme content in plants will contribute to produce increased leghemoglobin protein complex. The heme biosynthetic enzymes which are modified to be feedback-insensitive or are otherwise modified or edited to enhance enzyme expression, stability or activity are expressed in soybean seeds to further increase heme production, enabling higher leghemoglobin accumulation and heme loading in soybean seeds. Specifically, Glutamyl-tRNA reductase (GTR) enzyme activity is under combinatorial, post-translational control mediated by the proteins Fluorescent in Blue Light (FLU), Glutamyl-tRNA reductase-binding protein (GBP), chloroplast signal particle 43 (SRP43) (Table 4). Altered expression of a single or any combination of these three proteins achieved by gene editing, seed-preferred over-expression or RNA interference is expected to achieve higher level of heme-containing leghemoglobin by increasing heme-biosynthetic activity in developing seeds.

TABLE 3
Soybean Genes in the Porphyrin Pathway

| Enzyme name | Gene Model Name |
|---|---|
| Glutamyl-tRNA reductase | Glyma.02G218300 |
| | Glyma.04G089800 |
| | Glyma.06G091600 |
| | Glyma.07G184700 |
| | Glyma.08G064700 |
| | Glyma.14G185700 |
| glutamate-1-semialdehyde 2,1-aminomutase | Glyma.04G002900 |
| | Glyma.06G002900 |
| | Glyma.14G221900 |
| aminolevulinate dehydratase (HEMB1) | Glyma.04G247700 |
| | Glyma.06G115000 |
| hydroxymethylbilane synthase (HEMC) | Glyma.01G227400 |
| | Glyma.11G015400 |
| | Glyma.11G094700 |
| | Glyma.12G021100 |
| urophorphyrinogen III synthase | Glyma.04G037000 |
| | Glyma.06G037300 |
| urophorphyrinogen decarboxylase | Glyma.11G235400 |
| | Glyma.12G229700 |
| | Glyma.13G269900 |
| | Glyma.18G021500 |
| coporphyrinogen III oxidase (HEMF, CPOX) | Glyma.14G003200 |
| protoporphyrinogen oxidase (PPOX) | Glyma.10G138600 |
| | Glyma.02G007200 |
| | Glyma.19G245900 |
| | Glyma.08G173600 |
| ferrochelatase | Glyma.04G050400 |
| | Glyma.04G205600 |
| | Glyma.05G197600 |
| | Glyma.06G051100 |
| | Glyma.06G159900 |
| | Glyma.08G005000 |

TABLE 4
Soybean Genes Encoding Proteins that Regulate
Glutamyl-tRNA Reductase Activity

| Enzyme name | Gene Model Name |
|---|---|
| Glutamyl-tRNA reductase-binding protein | Glyma.08G222600 |
| chloroplast signal particle 43 | Glyma.11G097200 |
| FLUORESCENT IN BLUE LIGHT | Glyma.16G010200 |
| | Glyma.07G041700 |

### Example 4

This example demonstrates genome engineering of the leghemoglobin gene into the native soybean RUBISCO gene loci.

Ribulose-1,5-bisphosphate carboxylase-oxygenase (RUBISCO) is one of the most abundant proteins in plant leaves. For example, two RUBISCO genes and their expression levels in different plant tissues are listed in Tables 5. The genes encoding these proteins were used as the gene editing targets for soybean leghemoglobin over-expression in plant leaves as described in this example.

TABLE 5
Expression Profiling of RUBISCO Genes in Soybean

| | flower | leaf | root | seed | stem | SEQ IDs |
|---|---|---|---|---|---|---|
| Glyma.13g046200 | 2230 | 149584 | 66 | 813 | 2921 | SEQ ID 22 and 23 |
| Glyma.19g046800 | 9757 | 394066 | 463 | 2700 | 10343 | SEQ ID 24 and 25 |

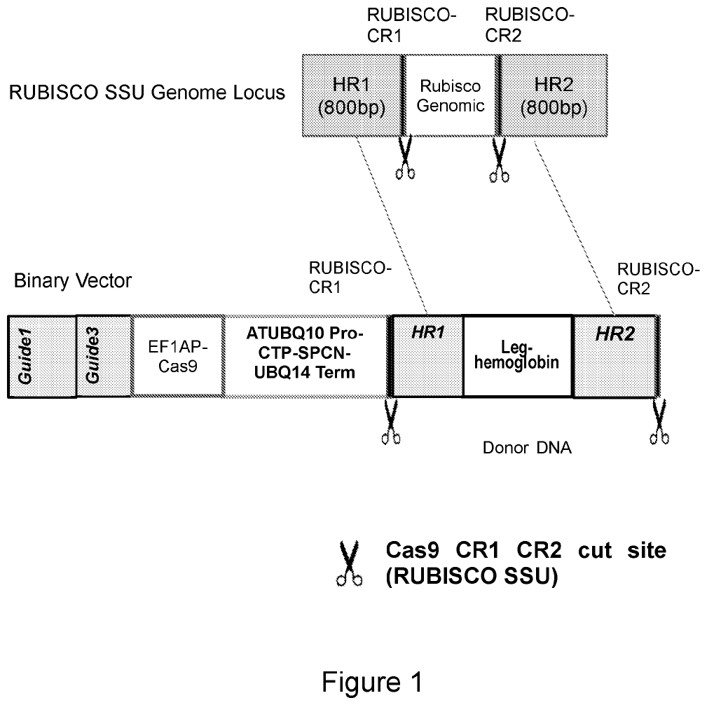

With the CRISPR/Cas9 system, specific gRNAs (GM-RUBISCO-CR1, SEQ ID NO: 16; and GM-RUBISCO-CR2, SEQ ID NO: 17) were designed to target the RUBISCO gene (glyma.13g046200, SEQ ID NO: 22 for genomic nucleotide sequences, SEQ ID NO: 23 for peptide sequences). The GM-RUBISCO-CR1 was designed to target a site near the beginning of the exon1 of the RUBISCO protein. The GM-RUBISCO-CR2 was designed to target a site near the end of last exon of the RUBISCO gene. As shown in FIG. 1, the binary vectors contained the CR1/CR2 gRNA combinations and their corresponding donor DNA templates (SEQ ID NO: 42). The homology recombination (HR) fragments were used to flank the leghemoglobin sequences to facilitate the homology-mediated recombination process. The CR1 or CR2 gRNA target sites were also used to flank the donor DNAs to enable them to be excised from the binary vectors for double strand break repair process. These sequences are defined in Table 6.

TABLE 6
Nucleotide Sequences of HR Fragments and
CR Cut Sites in Donor DNA Templates

| | Location in SEQ ID NOs |
|---|---|
| HR1 | Position 24 to position 823 of SEQ ID NO: 42 |
| HR2 | Position 1262 to position 2061 of SEQ ID NO: 42 |
| RUBISCO-CR1 | Position 1 to position 23 of SEQ ID NO: 42 |
| Cut Site | |
| RUBISCO-CR2 | Position 2062 to position 2084 of SEQ ID NO: 42 |
| Cut Site | |

The binary vectors are introduced into plants by Agrobacterium-mediated transformation. With site-specific integration of the donor DNA by homology-mediated double strand break DNA repair process, a genome editing variant was created by replacing the genomic sequences of the entire RUBISCO protein with the soybean leghemoglobin protein at the native RUBISCO gene locus. TO plants are generated and molecularly analyzed to identify the perfect gene integration variants. The leghemoglobin content in T0, T1 and homozygous T2 plants are analyzed as described in Example 7.

### Example 5

This example demonstrates genome engineering of the leghemoglobin gene into the native soybean vegetative storage protein gene loci.

There are other high abundant proteins in plant leaves, such as the vegetative storage proteins (VSP). Several VSP gene examples and their expression levels in different plant tissues are shown in Table 7. The genes encoding these proteins were used as the gene editing targets for soybean leghemoglobin over-expression in plant leaves as described in this example.

TABLE 7
Expression Profiling of Vegetative Storage Proteins in Soybean

| | flower | leaf | root | seed | stem | SEQ IDS |
|---|---|---|---|---|---|---|
| Glyma.07g014500 | 48765 | 115740 | 467 | 568 | 86675 | SEQ ID 26 and 27 |
| Glyma.08g200100 | 20810 | 13636 | 286 | 24 | 37693 | SEQ ID 28 and 29 |
| Glyma.08g200200 | 664 | 1362 | 98 | 40 | 49 | SEQ ID 30 and 31 |
| Glyma.15g218400 | 482 | 125 | 203 | 201 | 97 | SEQ ID 32 and 33 |
| Glyma.08g200000 | 6438 | 46 | 10 | 952 | 624 | SEQ ID 34 and 35 |

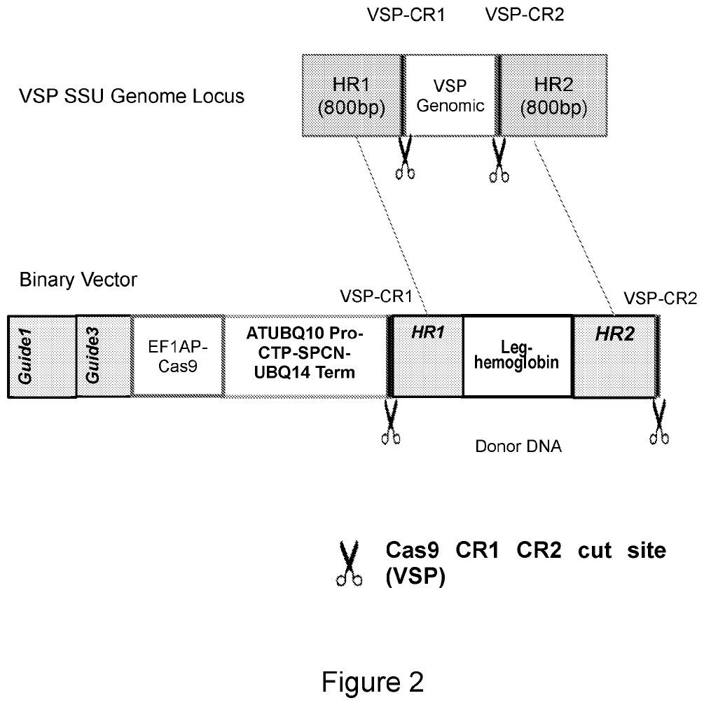

With the CRISPR/Cas9 system, specific gRNAs (GM-VSP-CR1, SEQ ID NO: 18; and GM-VSP-CR2, SEQ ID NO: 19) to target the VSP gene (glyma.07g014500, SEQ ID NO: 26 for genomic nucleotide sequences, SEQ ID NO: 27 for peptide sequences) were designed. The GM-VSP-CR1 was designed to target a site near the beginning of the exon1 of the VSP protein. The GM-VSP-CR2 was designed to target a site near the end of last exon of the VSP gene. As shown in FIG. 2, the binary vectors contained the CR1/CR2 gRNA combinations and their corresponding donor DNA templates (SEQ ID NO: 43). The homology recombination (HR) fragments were used to flank the leghemoglobin sequences to facilitate the homology-mediated recombination process. The CR1 or CR2 gRNA target sites were also used to flank the donor DNAs to enable them to be excised from the binary vectors for double strand break repair process. These sequences are defined in Table 8.

TABLE 8
Nucleotide Sequences of HR Fragments and
CR Cut Sites in Donor DNA Templates

| | Location in SEQ ID NOs |
|---|---|
| HR1 | Position 24 to position 823 of SEQ ID NO: 43 |
| HR2 | Position 1262 to position 2061 of SEQ ID NO: 43 |
| VSP-CR1 Cut Site | Position 1 to position 23 of SEQ ID NO: 43 |
| VSP-CR2 Cut Site | Position 2062 to position 2084 of SEQ ID NO: 43 |

The binary vectors are introduced into plants by Agrobacterium-mediated transformation. With site-specific integration of the donor DNA by homology-mediated double strand break DNA repair process, a genome editing variant was created by replacing the genomic sequences of the entire VSP protein with the soybean leghemoglobin protein at the native VSP gene locus. T0 plants are generated and molecularly analyzed to identify the perfect gene integration variants. The leghemoglobin content in T0, T1 and homozygous T2 plants are analyzed as described in Example 7.

### Example 6

This example demonstrates genome engineering of the leghemoglobin gene into the native soybean RUBISCO Activase gene loci.

There are other high abundant proteins in plant leaves, such as the RUBISCO Activase (RCA). Several RCA gene examples and their expression levels in different plant tissues are shown in Tables 9. The genes encoding these proteins were used as the gene editing targets for soybean leghemoglobin over-expression in plant leaves as described in this example.

TABLE 9
Expression Profiling of RUBISCO Activase Genes in Soybean

| | flower | leaf | root | seed | stem | SEQ IDs |
|---|---|---|---|---|---|---|
| Glyma.11g221000 | 1051 | 45465 | 70 | 966 | 234 | SEQ ID 36 and 37 |
| Glyma.02g249600 | 595 | 7145 | 41 | 218 | 374 | SEQ ID 38 and 39 |
| Glyma.14g067000 | 1170 | 2881 | 83 | 162 | 585 | SEQ ID 40 and 41 |

With the CRISPR/Cas9 system, specific gRNAs (GM-RCA-CR1, SEQ ID NO: 20; and GM-RCA-CR2, SEQ ID NO: 21) to target the RCA gene (glyma.11g221000, SEQ ID NO: 36 for genomic nucleotide sequences, SEQ ID NO: 37 for peptide sequences) were designed. The GM-RCA-CR1 was designed to target a site near the beginning of the exon1 of the RCA protein. The GM-RCA-CR2 was designed to target the end of last exon of the RCA gene. As shown in FIGS. 5, the binary vectors contained the CR1/CR2 gRNA combinations and their corresponding donor DNA templates (SEQ ID NO: 44). The homology recombination (HR) fragments were used to flank the leghemoglobin sequences to facilitate the homology-mediated recombination process. The CR1 or CR2 gRNA target sites were also used to flank the donor DNAs to enable them to be excised from the binary vectors for double strand break repair process. These sequences are defined in Table 10.

TABLE 10
Nucleotide Sequences of HR Fragments and
CR Cut Sites in Donor DNA Templates

| | Location in SEQ ID NOs |
|---|---|
| HR1 | Position 24 to position 823 of SEQ ID NO: 44 |
| HR2 | Position 1262 to position 2061 of SEQ ID NO: 44 |
| RCA-CR1 Cut Site | Position 1 to position 23 of SEQ ID NO: 44 |
| RCA-CR2 Cut Site | Position 2062 to position 2084 of SEQ ID NO: 44 |

The binary vectors are introduced into plants by Agrobacterium-mediated transformation. With site-specific integration of the donor DNA by homology-mediated double strand break DNA repair process, a genome editing variant was created by replacing the genomic sequences of the entire RCA protein with the soybean leghemoglobin protein at the native RCA gene locus. T0 plants are generated and molecularly analyzed to identify the perfect gene integration variants. The leghemoglobin content in T0, T1 and homozygous T2 plants are analyzed as described in Example 7.

### Example 7

This example demonstrates the characterization of soybean leghemoglobin expression in soybean leaves.

For the 6 transgenic constructs and 3 gene editing experiments as described in previous examples, the leghemoglobin contents in leaves of T0 plants, segregating T1 plants and homozygous T2 plants are analyzed as described below.

### Sample Preparation

Leaf samples of T0 plants, segregating T1 plants and homozygous T2 plants are lyophilized. The lyophilized leaf samples are placed in a Spex Certiprep ½×2″ polycarbonate vial with cap (cat #3116PC). A ⅜″ stainless steel ball bearing is added. Grinding is performed in a Spex Certiprep 2000 Geno/Grinder at 1500 strokes/min for three 30 second intervals with a 1-minute rest between each cycle.

Alternatively, samples are ground with a pestle, in the presence of liquid nitrogen, in a precooled mortar. The powders are then lyophilized for 48 h and kept at −20° C. in a desiccator until processed.

Moisture content determinations are performed according to American Oil Chemists Society (AOCS Official Method Ba 2a-38, modified for small samples) as follows: weigh powdered sample material (approximately 100 mg; to an accuracy of 0.1 mg) into a pre-weighed (and recorded) 13×100 mm glass tube VWR (53283-800) and weigh again, place samples into a forced air oven preheated to 130° C., allow material to dry for 2 h, remove tubes into a desiccator cabinet and allow to come to room temperature before weighing again, cap tube and save residual dried material for subsequent combustion analysis for protein (see below), store in a desiccator for further analysis.

### Total Protein Analysis

Protein contents are estimated by combustion analysis of the oven dried or lyophilized powders described above. Analysis is performed on a Flash 1112EA combustion analyzer (commercially available from Thermo) running in the N-protein mode, according to the manufacturer's instructions, using aspartic acid as the standard. The powdered samples, 30-40 mg, weighed to an accuracy of 0.001 mg on a Mettler-Toledo MX5 microbalance are used for analysis. Protein contents are calculated by multiplying % N, determined by the analyzer, by 6.25. Final protein contents are assumed to be at a dry basis for the oven dried material and on an as measured basis for the lyophilized material.

Calculation of Moisture Content. The as is moisture content of the tissues is determined after oven drying using the following formula:

$$Moisture = \frac{( wt . tube + tissue as is - wt . tube ) - ( wt . tube + tissue dry - wt . tube )}{( wt . tube + tissue as is - wt . tube )} \times 100$$

### Quantitation of Globin Protein by LC-MS-MS

The amino acid sequence of the globin protein (Table 1; SEQ ID 2) is assessed in-silico for potential trypsin digestion sites and the suitability of the resultant peptides for quantitative mass spectrometry. The following criteria are applied: the peptide is between 6 and 20 amino acids in length, the amino acids within the peptide are unlikely to undergo secondary modifications, the absence of sulfur containing amino acids, and solubility and iso-electric point.

Using these criteria, a number of potential peptides are identified. These are further analyzed using an online application available from Thermo Fisher Scientific at thermofisher.com/us/en/home/life-science/protein-biology/peptides-proteins/custom-peptide-synthesis-services/peptide-analyzing-tool.html. Based on the output of this application peptides are selected. The sequences of these peptides are subjected to a BLAST search using the NCBI Protein BLAST (protein-protein) program blast.ncbi.nlm.nih.gov/Blast.cgi?PROGRAM=blastp&PAGE_TYPE=BlastSearch&LINK_LOC=blasthome and are determined to be unique to the soybean globin sequence within the soybean (Glycine max) genome. The peptides are synthesized as follows:

Peptide 1:
SEQ ID NO: 45
K.ANGTVVADAALGSIHAQKA
[position 78-95 of SEQ ID NO: 2]
Peptide 2:
SEQ ID NO: 46
K.AITDPQFVVVK.E
[position 96-106 of SEQ ID NO: 2]

Where the “.” indicates the enzymatic digestion site and the parenthetical values denote the amino acid residue position relative to the N-terminal end of the mature globin protein.

Peptide stocks, at a concentration of 500 ppm, are prepared and stored as aliquots at −80° C. These stocks are used to further assess the suitability of the peptides for quantitative analysis. Peptide stocks are infused into the Mass Spectrometer (SCIEX 5500 Qtrap; SCIEX LLC, Redwood City, CA USA) to optimize the parameters for detection. Upon analysis, the best candidate is selected. Following optimization of fragmentation in the collision cell, a surrogate daughter ion with the highest abundance is chosen to develop quantitation against. A second confirmatory ion is also chosen.

### Sample Preparation

Powder samples of ˜10-20 mg (weighed and recorded to an accuracy of 0.1 mg) were placed into 1.2 ml Micro Titer Tubes (Fisher Brand 02-681-376). Extraction buffer, 8 mM (3-[(3-Cholamidopropyl)dimethylammonio]-1-propanesulfonate hydrate, (CHAPS); 0.1% Triton X-100, pH 8.4 was added at a tissue weight to volume ratio of 25. One small steel ball was added to each vial and after capping the samples were extracted in a Geno/Grinder; 1150 oscillations per minute for 30 seconds. The contents of the homogenization tube, minus the steel ball, were quantitatively transferred to clean 1.5 ml microfuge tubes and the samples were cleared in a microcentrifuge; 10,670×g for 10 minutes. The supernatants were transferred to clean 1.5 ml microcentrifuge tubes and the samples were again centrifuged; 10,670×g, for 5 minutes. Total soluble protein concentrations of the supernatants were determined using the Bradford assay and the results were used to normalize samples to 1 mg soluble protein per ml, by dilution with trypsin digestion buffer (100 mM Ammonium Bicarbonate; 0.05% Tween-20; pH 8.3). Samples were prepared for trypsin digestion by adding 50 ul of the protein normalized extract to 100 ul of trypsin digestion buffer, 6 ul of 0.25M DTT (dithiothreitol; in digestion buffer) and incubating them at 95° C. for 20 minutes. Iodoacetamide, 6 ul of 300 mM stock was added to each sample and they were incubated in the dark for one hour at room temperature. Trypsin (Pierce, MS Grade; Thermo Fisher Scientific) 10 ul of 0.1 ug/ul stock, was added to each sample and they were incubated overnight at 37° C. in a static incubator. The tryptic digestions were terminated by the addition of 10 ul of 10% formic acid. Samples were then analyzed using UHPLC-MS-MS analysis.

### LC/MS/MS Methods

Quantitative analysis of the tryptic digests is performed on a UHPLC (Agilent 1290) with SCIEX 5500 Qtrap detector, operating in the positive ion mode. Samples and standards (10 ul injections) are separated on a Waters Cortex C18, 2.7 um (2.1×100 mm) reverse phase column maintained at 40° C. The solvent flow rate is 300 ul/min with starting conditions of, 90% solvent A (99.9% MS grade Water; 0.1% Formic Acid)-10 solvent B (99.9% Acetonitrile, 0.1% Formic Acid). The conditions are ramped to 60% solvent A-40% solvent B over a 7-minute period, followed by a further ramp to 10% Solvent A-90% Solvent B over 0.5 min. The solvents are then returned to the starting conditions, over a 3-minute period and the column is equilibrated under the starting conditions for a further 3 minutes before the next injection. An Electrospray Ionization (ESI) source is used to introduced samples into the MS. Source parameters are as follows: Declustering potential 135 (V), Temperature 350° C., and Ion Spray voltage 350V. An MRM (Multiple Reaction Monitoring) detection technique is used to identify and quantitate the product ion (m/z: 816.6) using a collision cell energy of 35 (eV) to fragment the parent +2 molecule (m/z 608.9). Another product ion (m/z: 444.3) is used to confirm identity (based on the presence or absence). Quantitation is performed against a standard curve of the peptide that had been taken through all of the sample preparation steps described above.

In other experiments the UHPLC method is modified to accommodate samples with higher levels of globin expression. These changes can include: (1) reducing the injection volume from 10 ul to 2 ul; (2) shortening the elution profile (90% solvent A; 10% solvent B to 60% Solvent A; 40% Solvent B) to 5 minutes (from the original 7 min); (3) the second ramp, to 10% Solvent A-90% Solvent B is increased to 1 min (originally 0.5 min), (4) the ramp back to starting conditions (90% solvent A; 10% solvent B) is shortened to 0.5 min (from, for example, 3 min) and these conditions are maintained for 3.5 min to allow the system to fully equilibrate before the next sample injection.

To improve the extraction efficiency and make the sample preparation more uniform the sample preparation is modified as follows; powder samples of 10+/−0.5 mg (weighed and recorded to an accuracy of 0.1 mg) are placed into 1.2 ml Micro Titer Tubes (Fisher Brand 02-681-376). Extraction buffer, 8 mM (3-[(3-Cholamidopropyl)dimethylammonio]-1-propanesulfonate hydrate, (CHAPS); 0.1% Triton X-100, pH 8.4 is added at a tissue weight to volume ratio of 50. One small steel ball was added to each vial and after capping the samples are extracted in a Geno/Grinder; 1150 oscillations per minute for 30 seconds and then on an end over end rotator for 10 minutes; the genogrinding step is then repeated. The contents of the homogenization tube, minus the steel ball, are quantitatively transferred to clean 1.5 ml microfuge tubes and the samples are cleared in a microcentrifuge; 10,670×g for 10 minutes. The supernatants are transferred to clean 1.5 ml microcentrifuge tubes and the samples are again centrifuged; 10,670×g, for 5 minutes. Total soluble protein concentrations of the supernatants are determined using the Bradford assay and the results are used to normalize samples to 1 mg soluble protein per ml, by dilution with trypsin digestion buffer (100 mM Ammonium Bicarbonate; 0.05% Tween-20; pH 8.3). Samples are prepared for trypsin digestion by adding 25 ul of the protein normalized extract to 125 ul of trypsin digestion buffer, 6 ul of 0.25M DTT (dithiothreitol; in digestion buffer) and incubating them at 95° C. for 20 minutes. Iodoacetamide, 6 ul of 300 mM stock is added to each sample and they are incubated in the dark for one hour at room temperature. Trypsin (Pierce, MS Grade; Thermo Fisher Scientific) 10 ul of 0.1 ug/ul stock, was added to each sample and they were incubated overnight at 37° C. in a static incubator. The tryptic digestions are terminated by the addition of 10 ul of 10% formic acid. Samples are then analyzed using UHPLC-MS-MS analysis.

The modified extraction method is expected to result in an average of 97% (range 95.5-100%) of the soluble protein being extracted in the first extraction. This would represent an average of 71% (range 62-78%) of the total protein content of the extracted material.

### Example 8

This example demonstrates the genome engineering of the leghemoglobin gene into the native loci in alfalfa, maize, rice and other plant leaves.

The technical approaches described in examples 1 to 6 are adapted for leghemoglobin expression in leaves of alfalfa, maize, rice or other plants by site specific leghemoglobin integration into high expressed gene loci, such as the RUBISCO gene, VSP gene, RUBISCO activase gene, or other highly expressed genes. Leghemoglobin is expressed in the leaves and the leghemoglobin content is extracted and measured according to Example 7 and is expected to be in the range of 0.01% to 10% of the total leaf protein.

### Example 9

This example demonstrates the genome engineering of the leghemoglobin gene into the native loci in soybean alfalfa, maize, rice and other plant roots.

The technical approaches described in examples 1 to 6 are adapted for leghemoglobin expression in roots of soybean, alfalfa, maize, or rice by site specific leghemoglobin integration into high root expressed gene loci, such as sulfur transporter genes (Yoshimoto 2002), phosphate transporter genes (Mudge 2002, Koyama 2005), ribonuclease LX gene (Kock 2006), and Lysyl-tRNA-synthetase-like protein (Giritch 1997). Leghemoglobin is expressed in the roots and the leghemoglobin content is extracted and measured according to Example 7 and is expected to be in the range of 0.01% to 10% of the total root protein.

### Example 10

This example demonstrates the genome engineering of the leghemoglobin gene into the native constitutive expressed gene loci for production in all parts of plants, including plant leaves, roots, seeds, fruits and tubers

All the technical approaches described in Examples 1-9 can be adapted for leghemoglobin expression in all parts of plants, including leaves, roots, seeds, fruits and tubers, by site specific leghemoglobin integration into constitutive expressed gene loci, such as ubiquitin genes (Hernandez-Garcia 2009), translation elongation factors (EF1a) genes (U.S. Pat. No. 8,710,206), and actin gene (Meagher 1999). Leghemoglobin is expressed in all plant parts. The leghemoglobin content is extracted and measured according to Example 7 and is expected to be in the range of 0.01% to 10% of the total plant protein.

### Example 11

This example demonstrates the expression of soybean leghemoglobin in Alfalfa leaves

The expression vectors described in the Example 1 were introduced into alfalfa by agrobacterium-based transformation. The transgenic plants were molecularly characterized to select single copy events. The leghemoglobin contents in the leaves were characterized (Table 11) by methods described in the Example 7.

When the RCA2 promoter was used, the vector that did not contain a signal peptide coding sequence gave a high expression of leghemoglobin; the leghemoglobin protein content ranged from 0.012% to 0.028% on leaf dry weight basis or from 0.028% to 0.065% on total leaf protein basis. When the RUBISCO SSU chloroplast signal peptide was used with the RCA2 promoter, the leghemoglobin was either undetectable or extremely low, and ranged from 0.001% to 0.002% on leaf dry weight basis or from 0.002% to 0.005% on total leaf protein basis. The ER-targeting KDEL sequence gave intermediate results with the RCA2 promoter; leghemoglobin content ranged from 0.007% to 0.015% on leaf dry weight basis or from 0.016% to 0.035% on total leaf protein basis.

The RUBISCO SSU promoter gave similar results as the RCA2 promoter. When the RUBISCO SSU promoter was used, expression with the no signal peptide vector resulted in high expression of leghemoglobin, with leghemoglobin protein content ranging from 0.009% to 0.010% on leaf dry weight basis, or from 0.025% to 0.027% on total leaf protein basis. When the RUBISCO SSU promoter was used with the RUBISCO SSU chloroplast signal peptide, the leghemoglobin was either undetectable or 0.001% on leaf dry weight basis, or from 0.003% to 0.004% on total leaf protein basis. The ER-targeting KDEL sequence gave intermediate results with the RUBISCO SSU promoter; leghemoglobin content ranged from 0.004% to 0.005% on leaf dry weight basis, or from 0.011% to 0.013% on total leaf protein basis.

Our results demonstrate leghemoglobin accumulated up to 0.028% on leaf dry weight basis or 0.065% on total protein dry weight basis in alfalfa leaves. The non-targeted leghemoglobin provided higher leghemoglobin accumulation in the leaves.

Alfalfa is a multi-harvest crop per year, with an average of 4 tons/acre yield at about a 20% dry weight basis. The accumulation of 0.028% of leghemoglobin on a leaf dry weight basis can produce around 203 g of leghemoglobin per acre. With various numbers of cropping per year, ranging from 2 to 10 times per year based on the geographical locations, about 400 g to 2000 g of leghemoglobin can be produced per acre per year. As a comparison, with 0.5% leghemoglobin accumulation of soybean proteins in soybean seeds, with an average soybean yield at 51.4 bushel per acre, about 2436 g of leghemoglobin can be produced in soybean seeds per acre. Expression of leghemoglobin in plant leaves provides an alternative approach for leghemoglobin production in plants. Additionally, a combination of leghemoglobin production in both leaves and seeds in plants, such as soybean, alfalfa, and pea, will provide even more economic value.

TABLE 11
Quantification of Leghemoglobin content in alfalfa leaves

| | | | % globin | % globin |
|---|---|---|---|---|
| Sample | | Targeting | of leaf dry | of total |
| # | Promoter | sequence | weight (DW) | protein (DW) |
| 1 | RCA2 | No signal | 0.021 | 0.049 |
| 2 | Promoter | sequence | 0.028 | 0.065 |
| 3 | | | 0.018 | 0.042 |
| 4 | | | 0.021 | 0.049 |
| 5 | | | 0.012 | 0.028 |
| 6 | RCA2 | RUBISCO | Below detection | Below detection |
| 7 | Promoter | SSUSP | 0.002 | 0.005 |
| 8 | | | 0.001 | 0.002 |
| 9 | | | Below detection | Below detection |
| 10 | RCA2 | KDEL | 0.015 | 0.035 |
| 11 | promoter | | 0.015 | 0.035 |
| 12 | | | 0.011 | 0.026 |
| 13 | | | 0.014 | 0.033 |
| 14 | | | 0.011 | 0.026 |
| 15 | | | 0.007 | 0.016 |
| 16 | RUBISCO | No signal | 0.009 | 0.025 |
| 17 | SSU | sequence | 0.010 | 0.027 |
| 18 | promoter | | 0.010 | 0.026 |
| 19 | RUBISCO | RUBISCO | Below detection | Below detection |
| 20 | SSU | SSUSP | 0.001 | 0.003 |
| 21 | promoter | | 0.001 | 0.003 |
| 22 | | | 0.001 | 0.004 |
| 23 | | | Below detection | Below detection |
| 24 | RUBISCO | KDEL | 0.004 | 0.010 |
| 25 | SSU | | 0.005 | 0.013 |
| 26 | promoter | | 0.004 | 0.011 |
| 27 | | | 0.004 | 0.013 |
| 28 | | | 0.004 | 0.012 |
| 29 | | | 0.004 | 0.011 |

All publications and patent applications in this specification are indicative of the level of ordinary skill in the art to which this invention pertains. All publications and patent applications are herein incorporated by reference to the same extent as if each individual publication or patent application was specifically and individually indicated by reference.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

1. A plant leaf comprising a genomic modification, the genomic modification comprising one or more of (i) an insertion of a leghemoglobin coding sequence into a native non-leghemoglobin gene such that the leghemoglobin coding sequence replaces all or part of the native non-leghemoglobin gene coding sequence, (ii) an insertion of a heterologous regulatory enhancer or heterologous promotor sequence operably linked to a native leghemoglobin coding sequence, and (iii) a substitution or deletion wherein the substitution or deletion creates or enhances a regulatory enhancer or promotor sequence of a native leghemoglobin coding sequence, wherein a leghemoglobin protein is expressed in the plant leaf.

2. The plant leaf of claim 1, wherein the native leghemoglobin coding sequence encodes a polypeptide having at least 90% identity to SEQ ID NO: 2.

3. The plant leaf of claim 1, wherein the leghemoglobin protein is expressed in an amount of at least 0.05% of the total leaf protein.

4. The plant leaf of claim 1, where native non-leghemoglobin gene is selected from the group consisting of a RUBISCO gene, a vegetative storage protein, a RUBISCO Activase gene, or any combination thereof.

5. The plant leaf of claim 1, wherein the insertion further comprises a targeting sequence operably linked to the inserted leghemoglobin coding sequence or the native leghemoglobin coding sequence, wherein targeting sequence targets the leghemoglobin to the plastid or endoplasmic reticulon and comprises a polynucleotide encoding a polypeptide having at least 95% identity with SEQ ID NO: 53 or 54.

6.-7. (canceled)

8. The plant leaf of claim 1, wherein the plant leaf further comprises a recombinant construct comprising one or more porphyrin pathway genes operably linked to a heterologous regulatory element, wherein the plant leaf has been modified to have one or more nucleotide insertions, deletions, or substitutions to generate a plant leaf comprising the one or more porphyrin pathway genes operably linked to a heterologous regulatory element and wherein the porphyrin pathway gene is selected from the group consisting of a glutamyl-tRNA reductase, a ferrochelatase, a glutamate-1-semialdehyde 2, a 1-aminomutase, an aminolevulinate dehydratase, a hydroxymethylbilane synthase, a urophorphyrinogen III synthase, a urophorphyrinogen decarboxylase, a coporphyrinogen III oxidase, and a protoporphyrinogen oxidase, or any combination thereof.

9.-11. (canceled)

12. A plant leaf comprising a leghemoglobin protein in an amount of at least 0.1% of total protein in the plant leaf, wherein the plant leaf comprises a leghemoglobin coding sequence operably linked to a heterologous regulatory element.

13. The plant leaf of claim 12, wherein the plant leaf comprises a recombinant construct comprising the leghemoglobin coding sequence operably linked to the heterologous regulatory element and a targeting sequence operably linked to the leghemoglobin coding sequence, the targeting sequence targeting the leghemoglobin to the plastid or endoplasmic reticulum and comprising a polynucleotide encoding a polypeptide having at least 95% identity to SEQ ID NO: 53 or 54.

14.-18. (canceled)

19. The plant leaf of claim 18, wherein the plant leaf genome has been modified to introduce (i) an insertion of a heterologous regulatory enhancer or heterologous promoter sequence operably linked to the native leghemoglobin coding sequence, or (ii) a substitution wherein the substitution creates or enhances a native leghemoglobin coding sequence regulatory enhancer or a native leghemoglobin coding sequence promotor sequence.

20. The plant leaf of claim 18 or 19, wherein the plant leaf genome has been modified to introduce an insertion, and the insertion comprises a targeting sequence operably linked to a leghemoglobin coding sequence of the leghemoglobin gene, the targeting sequence targeting the leghemoglobin to the plastid or endoplasmic reticulum and wherein the targeting sequence comprises a polynucleotide encoding a polypeptide having at least 95% identity with SEQ ID NO: 53 or 54.

21-22. (canceled)

23. The plant leaf of claim 12, wherein the plant leaf genome has been modified to replace all or part of a coding sequence of a non-leghemoglobin gene with a leghemoglobin coding sequence, wherein the non-leghemoglobin gene is a RUBISCO gene, a RUBISCO Activase gene, or a vegetative storage protein gene.

24. (canceled)

25. The plant leaf of claim 23, wherein the plant leaf genome has been modified to introduce an insertion, and the insertion comprises a targeting sequence operably linked to the leghemoglobin coding sequence which targets the leghemoglobin to the plastid or endoplasmic reticulum and comprises a polynucleotide encoding a polypeptide having at least 95% identity with SEQ ID NO: 53 or 54.

26.-27. (canceled)

28. The plant leaf of claim 12, wherein the plant leaf further comprises a recombinant construct comprising one or more porphyrin pathway genes operably linked to a heterologous regulatory element or wherein the plant leaf has been modified to have one or more nucleotide insertions, deletions, or substitutions to generate a plant leaf comprising the one or more porphyrin pathway genes operably linked to a heterologous regulatory element, wherein the porphyrin pathway gene is selected from the group consisting of a glutamyl-tRNA reductase, a ferrochelatase, a glutamate-1-semialdehyde 2, a 1-aminomutase, an aminolevulinate dehydratase, a hydroxymethylbilane synthase, a urophorphyrinogen III synthase, a urophorphyrinogen decarboxylase, a coporphyrinogen III oxidase, and a protoporphyrinogen oxidase, or any combination thereof.

29.-31. (canceled)

32. The plant leaf of claim 1, wherein the plant leaf does not contain (i) a recombinant construct comprising a sequence encoding a glutamyl tRNA reductase, or a truncated portion thereof, (ii) a recombinant construct comprising a sequence encoding a ferrochelatase, (iii) a recombinant construct comprising a sequence encoding a glutamyl tRNA reductase binding protein, and (iv) a recombinant construct comprising a sequence encoding an aminolevulinic acid synthase.

33. The plant leaf of claim 12, wherein the leghemoglobin coding sequence encodes a polypeptide having at least 95% identity to SEQ ID NO: 2.

34.-44. (canceled)

45. The plant leaf of claim 1, wherein the plant leaf is a soybean leaf, a pea leaf, a maize leaf, an alfalfa leaf, or a rice leaf and wherein the plant leaf comprises a leghemoglobin complex comprising the leghemoglobin protein associated with a heme group.

46. (canceled)

47. A plant comprising the plant leaf of claim 1.

48.-74. (canceled)

75. A method for extracting leghemoglobin protein from the plant leaf of claim 1, the method comprising contacting the leaf with at least one of a cellulase, a hemicellulase, and a pectinase under conditions sufficient to degrade the polysaccharides in the leaf and filtering the permeant from the residue.

76. An isolate produced from the plant leaf of claim 1, the isolate comprising at least 0.2% leghemoglobin by weight of total protein, wherein at least about 50% of the leghemoglobin is hemelated with an iron group.

77. A protein fraction extracted from the plant leaf of claim 1, wherein the protein fraction comprises at least 0.1% leghemoglobin by weight of total protein.


SEQUENCE LISTING (54 sequences)

SEQ ID NO: 1 - DNA, 438 bp, Glycine max
  source 1..438: mol_type = genomic DNA
atgggtgctttcactgagaagcaagaggctttggtgagtagctcattcgaagcattcaaggcaaacattcctcaatacagcgttgtgttctacacttcgatactggagaaagcacccgcagcaaaggacttgttctcgtttctatctaatggagtagatcctagtaatcctaagctcacgggccatgctgaaaagctttttggattggtgcgtgactcagctggtcaacttaaagcaaatggaacagtagtggctgatgccgcacttggttctatccatgcccaaaaagcaatcactgatcctcagttcgtggtggttaaagaagcactgctgaaaacaataaaggaggcagttggggacaaatggagtgatgaattgagcagtgcttgggaagtagcctatgatgaattggcagcagctattaagaaggcattttag

SEQ ID NO: 2 - protein, 145 aa, Glycine max
MGAFTEKQEALVSSSFEAFKANIPQYSVVFYTSILEKAPAAKDLFSFLSNGVDPSNPKLTGHAEKLFGLVRDSAGQLKANGTVVADAALGSIHAQKAITDPQFVVVKEALLKTIKEAVGDKWSDELSSAWEVAYDELAAAIKKAF

SEQ ID NO: 3 - DNA, 5214 bp, Glycine max
  source 1..5214: mol_type = genomic DNA
tctaactaagtaagaaattgaagtattttataaaaaaaaattgactcatcgaatttataaagtaatttatgtaacaaaaattaagagtaatttaaaagcaatacatagatgaataggagtgtttttattaggaagaataagagtgttaataatatcacacgtgaacctcttaaatcaagtaagctaaatctctttggtaaaattaagattcacctacattgtttttttttttttatcaatacctacatcaatataaaaacaataattttttatcaatacctacattaatataaaaaaaataaatgatacagttacattaaatattcagagtaaaagctttgacactcatttatttttatctaactcaaatataattttctctattaaataatatttatggtctaaattttttttagctatattgcagctcgagtcttgatatcatgacatcttagccaactaaagctcaactggctcaaaggcaggaatatttcaaaaaaaattatttcatatatattaatatattgattaagaaaataaaaaaattatactaaaagaaccatgactttacccagcaccaaggtgttcaattttcaaaagccacaccactggaagaatgagaagccaacatagtctttcatggactaacacatccacaaacgtcacggttgtctttcatgcaccacacgaaattgcctttttctttattcttcaagaaattatggcatcaattagtcttaatagtcaaattcgtaaaaactaacaaattgacttttaaaaaattaaaaaattcaaattaatcacgggatatgtataaattatgatagactaatcattgtaaataagttaatgataaattctcaaaaagataatttatttttaaattaattattaaacattataactaaataataaaatgatctcacaaattaaatgacatgactaatttgttatattttttcatatttgaaaactacaattttatgtcatttttatggattaatttatatacttttaagaataaatttaactatttactccataaattatcgattattgatgtttactaattttgaagtttttgctaacaagtgtcgtcctaaagtaatttaattttcactcattaacaaaaaaataatttaagaattataaaattagtttaataattgaaacgagaagaaaggaagaacagattgtgagttacagtctctttgtcccctaacaattaatatttaatgataaaaaatagtaatttgactgttaaaaattataagaaagaaaataatatattttataaattctttagtatccgaattgtatatatatttattcaatactgattgatacaattcatattaatataatcgagtttttactgaacatacatttattaaaaaaaatctctctagtgtccatttattcggcgagaagccttctcgtgctttacacactttaatattattatatccccacccccaccaaaaaaaaaaaaactgttatatctttccagtacatttatttcttttatttttacaaaggaaacttcacgaaagtaattacaaaaaagatagtgaacatcatttttttagttaagatgaattttaaaatcacacttttttatatttttttgttacccttttcattattgggtgaaatctcatagtgaaactattaaatagtttgggctcaagttttattagtaaagtctgcatgaaatttaacttaataatagagagagttttggaaaggtaacgaatgttagaaagtgtgatattattatagttttatttagattaataattatgtttacatgaaaattgacaatttatttttaaaattcagagtaatacttaaattacttatttactttaagattttgaaaagatcatttggctcttcatcatgccgattgacaccctccacaagccaagagaaacttaagttgtaatttttctaactccaagccttctatataaacacgtattggatgtgaagttgttgcataacttgcattgaacaatagaaataacaacaaagaaaataagtgaaaaaagaaatatgggtgctttcactgagaagcaagaggctttggtgagtagctcattcgaagcattcaaggcaaacattcctcaatacagcgttgtgttctacacttcgtaagttttctcttaaagcatgtatctttcattctctgtttttcctttcgacattttttgtgtttgaaaagagatagtgtcaatgtgagtgggtatttttttttattaaaaattaacaggatactggagaaagcacccgcagcaaaggacttgttctcgtttctatctaatggagtagatcctagtaatcctaagctcacgggccatgctgaaaagctttttggattggtaagtatcatccaactaaaattatagctattttatgtgattaattttaagattaaacatgtatttaacactcttaaacatgtatttaacactcttaagattaaacatgtatttaactaaaacatgtatttgctgattattttttttttataattatcttgtcacatattatatattttttgaattgtaggtgcgtgactcagctggtcaacttaaagcaaatggaacagtagtggctgatgccgcacttggttctatccatgcccaaaaagcaatcactgatcctcagttcgtggtatgataaataataaaatgttacaataaatgcacatatacttaaattttacatggtgcagtgttatgatcatcatttttgtttagtaatgaatttacttaaaatcttaaattatgtactttttgaaagttttatatggaattttaattatagggaaaaatgtaagagctaatccattagtgatgttttgtctgtaggtggttaaagaagcactgctgaaaacaataaaggaggcagttggggacaaatggagtgatgaattgagcagtgcttgggaagtagcctatgatgaattggcagcagctattaagaaggcattttaggatctactattgccgtcaagtgtaataaataaattttgtttcactaaaacttgttattaaacaagtccccgatatataaatgttggttaaaataagtaaattatacggtattgataaacaatcttaagttttatatatagttccatatactaaagtttgtgaatcataatcgatgctttatataaataagttcataagaatttatatgttttaaaattattttacccctataaattcaaataaacttaactaaatgggatagatgttttcctacgaaaaaaatgaaaataagtaacagtaaaatgcgttagaaatagttattgtggtagataaattcaaatacattgaaaatatttttatcaattagattaatatgccaatgtttctgattttattactttaaagaatagcaggggtaagagggggattagcactcaaaaactgacatgaggccagaatgttaccatggtagcgcaatcgatcaactattaacatatacagtaaatctaaaagattaagggttcgtttcgtagaattaaaaaaaataattcaagttttgaattaaaatatgatttaaaatgttaattctatattttttttctttttcactttctttctctacaaatgagaaattagaagctcaattccattgcacggtatactcaaaccataattacaatttatcacgcgtaaatagcagaacgtacgggttgctgagagactaacaaagatattcaaggtccaatttgttatacatttccgccactgatgaaagaacattactatgcaaattccttatagaagaaaacatttacccttctcaattctcatttgcagcaaaatcacactctttaagctaattgattgattaaactggtgtagaattataaataaatcaataaaatgagatcattttgaacaaagtgaagttgatttctcacttcacaaagttcgccaatactgatggtaacttttaggtgtgaaataagatgggctgaataaagttttgaataaattggtttcatgagcctatttgaaaaatggtttgttttgtgtgagcgttttatttaggagaatgacgaaacacatatttcgccaagagaaataatgaaataggtgcatatttcgtcaataggaacaacgaaataggtgtatgttaggtcaggtgcagtgtgcagagtggattttgtcatccttattggcgaaataggtggtgctgttgctttgtcaatttatctaatagtgagttgaaaataatttatctaataatgagtcagaaataatttatctaataatgagtcaaaaccgccaaaaaatccacaaagcagcaccacttatttcgccaatgaggatggcaaaatccactctgcatgctgcacttatttcgccaatgtggatggcgaaatccactctgcacactgcacccctacgcgctgcatactgctgcacctgacctgacctacacctatttcgctattccggttggcgaaatatgcacctatttcactgttcctcttggcgaaatatgtgtttcgccattcctttcagcaaaacgctcacacaaaacagaccacattagcaaaatgtttcaaaacagattgggaattagtttctaaaagaaaccccccgaagtcaatttgctataaaatttaaacttatttaaataattgagtgcaactgtaaacttaaatgtatttttttttgtttaaacatataagtacacaaataatgaatccaaatataataaattcactaataactcaactcggtcatctttgaaatcatgaatatatttattatatctatacataatccaacaattatataatgttttatttaacaaacagtgattaatgtattgaaattaaaattaaatcattcaggtaataccaaaattcaatcattagcagaaataatttttgatcagattttatttacttgttcgctaaattttaaattagttagagtttcttttctttaatgaaccggagggtcaagataaaaaaatacattttttttttaataattatcatattataacaggtagttatttcaaagcaattcaatgcttcatttcaaccaccaacatttaaaagaaataaaatacaaaatagtactcgtggttttgtgtctggaaagctccaaccataatttaatccaaaagttgtctattgtaactttagcttaaataaaccatggtttaagactttca

SEQ ID NO: 4 - DNA, 603 bp, synthetic construct
  source 1..603: mol_type = other DNA
atggcttcctcaatgatctcctccccagctgttaccaccgtcaaccgtgccggtgccggcatggttgctccattcaccggcctcaaatccatggctggcttccccacgaggaagaccaacaatgacattacctccattgctagcaacggtggaagagtacaatgcatgggtgctttcactgagaagcaagaggctttggtgagtagctcattcgaagcattcaaggcaaacattcctcaatacagcgttgtgttctacacttcgatactggagaaagcacccgcagcaaaggacttgttctcgtttctatctaatggagtagatcctagtaatcctaagctcacgggccatgctgaaaagctttttggattggtgcgtgactcagctggtcaacttaaagcaaatggaacagtagtggctgatgccgcacttggttctatccatgcccaaaaagcaatcactgatcctcagttcgtggtggttaaagaagcactgctgaaaacaataaaggaggcagttggggacaaatggagtgatgaattgagcagtgcttgggaagtagcctatgatgaattggcagcagctattaagaaggcattttag

SEQ ID NO: 5 - protein, 200 aa, synthetic construct
MASSMISSPAVTTVNRAGAGMVAPFTGLKSMAGFPTRKTNNDITSIASNGGRVQCMGAFTEKQEALVSSSFEAFKANIPQYSVVFYTSILEKAPAAKDLFSFLSNGVDPSNPKLTGHAEKLFGLVRDSAGQLKANGTVVADAALGSIHAQKAITDPQFVVVKEALLKTIKEAVGDKWSDELSSAWEVAYDELAAAIKKAF

SEQ ID NO: 6 - DNA, 450 bp, synthetic construct
  source 1..450: mol_type = other DNA
atgggtgctttcactgagaagcaagaggctttggtgagtagctcattcgaagcattcaaggcaaacattcctcaatacagcgttgtgttctacacttcgatactggagaaagcacccgcagcaaaggacttgttctcgtttctatctaatggagtagatcctagtaatcctaagctcacgggccatgctgaaaagctttttggattggtgcgtgactcagctggtcaacttaaagcaaatggaacagtagtggctgatgccgcacttggttctatccatgcccaaaaagcaatcactgatcctcagttcgtggtggttaaagaagcactgctgaaaacaataaaggaggcagttggggacaaatggagtgatgaattgagcagtgcttgggaagtagcctatgatgaattggcagcagctattaagaaggcatttaaggacgagctgtga

SEQ ID NO: 7 - protein, 149 aa, synthetic construct
MGAFTEKQEALVSSSFEAFKANIPQYSVVFYTSILEKAPAAKDLFSFLSNGVDPSNPKLTGHAEKLFGLVRDSAGQLKANGTVVADAALGSIHAQKAITDPQFVVVKEALLKTIKEAVGDKWSDELSSAWEVAYDELAAAIKKAFKDEL

SEQ ID NO: 8 - DNA, 1745 bp, Glycine max
  source 1..1745: mol_type = genomic DNA
tcagaggaacctattatgaagcacttatcttgccgacataggctttctgctctccactaatctttcgaaacttcattttgtcacatgttactaagttgaaaaagaaaatattgttttagaagctaaggatgaaatagtagctaaaggaaagtgatatagaatttgtagtaaaatggaagagtgtaaagctatttactatttgttatttgctttagtacatagaatggataactcaagtgctggttccattaccaaatttcagtggttttggttgaatatggaatacagtggaagtggtgcagaagaggggagggaatgtttggggtacatgatttgaaaaaaggggagaatgattagaagagatagaaaattggcagaccaacattgaaggattcataggaatgttgtgtgttagtaaattggacaaatgttgctcttgatattttcagcatatcattcatcttagaagcccaactgttcttttttatatatattctctgttgccttcctttccttcaaaacacggcttttgaattttgaaccattaactgagttggaatagtggtaaaatcatgtagatttttcttttcgttttgtggggatatgaatacgaaatatccttacaaccagacaaagctgtggctcagaagaaagagaaacatgcctcactgctagaatcactagaactcacatcttgtatacaaactgttataccaaactgagaattttacaatttccaaagatgcaattctcaaccattaaccaatttcatcttaaacactaacagttcttcagtcttgaactttccatgctgaaaatttgttgcctatttcttttataggcagtggcttcttatgagagtcacactctctgaacttcttacgtaatacttgacttttcattttgtgctttataattgttatctcatgcattgcacttttaagataaggttgttgtgcattgtttggttgagtgcaacgatttactgattaccatagctacttgtggatcttatattttcaaaagtgtatgcttggtcattaatcaaccaagcatgataagccccatcatctaccaccccttctataccatatacgcattttgcttatctacattgctactgttacactcaaacaagtctaacagataatacgttaagttaatgaacatttttagtaatattattataaggattggccaatgtaattgtgaagagagaagcatgctttaagctacccaacaaaatggataagagtttcagttgatatggttctcttgtttcttttccaataaaaaaccaactttaaaatataaaatttactgtaacaaaggaacaaagagttttcacttaatccatgaatgagaaaggatggtcacaaaatatgttaggttaatatggaatgagggcactgtgcaaactacacaaataatatcaattccaccaccatcacacatttacgttcttccaaggaagagataagataatggagcctccacgtgtcacctccacatggtacctaacaataaggctaccattcaaaattttcctcactcgtgtggcctatatgctgtaatgtcatcacttattcaatccaacggttgtaacttttcggcaaccaatcctctccatttcacaccattggattagtactacacaaatcacactattatatatagcaagtttgagcagaagcttggatatctggcagcagaaaaacaagtagttgagaactaagaagaaggaa

SEQ ID NO: 9 - DNA, 2438 bp, Phaseolus vulgaris
  source 1..2438: mol_type = genomic DNA
tgaatgatgtaattaggtgataaaagcacactatattcttatgatataaaaagataaagtagatttgtagaagtctaatgtcttgttatttttcctcttctttcccaaagataaggcatgcaaatactctcatgcaaacaacacttttctccatgcacaatcaaactaattttccagacattttactcttatattgcaatgtcatatctcaggttgtcaacttttacaaacaattcgagttactgtaaatttgtatcactattggagatttttctaccaactttttacaaaacaactatagtgaataaatgtgttattttttatataatgtattataaaaaaactgagataacaaaagaaaaaaaataaagaatatgaaaaaaaaaatatttcttctattttcttttctttacatcaatcaaaatagagagccttaatagattcaagctgtacattcattttagtctagataaaagataaaatataaaacaaagagacaataaaaaataaaattaaaaaagtaaaataaaataaagactagacatatatacaaagagaaacaactaagaattataaaagataaaattaatattaattttcttataacaaccttacatccatattctttgttacaatgttatcaataaaaaaaataaaaaaaataattaaattaaatcaaaataataagtaaaattgaatatgttttagacacattcatttatataatataatatgaaatttattttaactttaaaccatctaaatacttagtacgaaaagaattggaataggttattatgtaggtttaggttttaaaggttaggatttagtataaaaataattgaaataggttattctatgtgtttaggtttttttttatcatcaaagaatgaaattaaattaagagggatacaaaaggggtatccccacccttttacataaaccagaccacattaagaatctccaagaataaaaagatgaacaaacatcccagaaacaatagaccgataatgttttctgcataagctagacattctatatgtttaggttttcaaagttaggatatatgattgaaatgagaaaatgaaaatattaaacaagcttatattttagataattaaacatcaaataatcaattttttgtagttttaaatttgtttcttgttatttggaagcttaacagtgaagaaaaagcagtgaagttgtgaatttcctttagttgcaagtgaagaatgttggccacacagaacagatagaagaaagaaggagagagacatggtagggtcaagggatgtaatagggctcaatccccttcattcatgaaattatctgagactcaccaattaaatttcttgtttctaccaacaataattcatcttcaaatgaacccattggctccaatgtggggtcattagtaccatctctacctctgtaaaaagacaacctcaaaaattatgccacttaccaattctaatattattactcttatgattggttatcaagattgaagatgcttgaatttgaataaacgacacaagttcttcaaggcttgtgtggcaaacaaatctccttaattctggtggagatagaaaaagctgcaatgacaacctcaaaatcctcgactaagagggacaaaaaaccttggaggcacagtatcttgccacgtggctgctgttcaatcttctatgtgtggatcttaggcctctgtgaccaatccttttgagcacaggcaactactataagtcagacgtacccttcgcttatactaagccatccaccactgaatttgcaactcaacaacaaccttaccctttctctccacaacagcacttccagatctttgtacgatatctttcctactttcttacactctttcccttttcatgtcttctttgatatccatctttatctagcatcatggtaatcattgcatgatgaatgctcaagtgttaggaaatgtaaaaacaagtaggctcgcacatgtttattcagctcacgtaaccctctgtcacttccaaacaagggttactatatcttcattttatgtgtcatagttgtaaagtgtgtcatggttgtatagttacatgctgagtttaaatatatgagtttcggactcttatgttcatgttggatatacatatcgaatatcgacatcctgatattagtaggacacgtatcggtgaagtatccaattcaaaaaatatttattgaatttataataattttagttcggttctaacataatttaaaaaaaaaatacattaatcttcttaaaacttaaacttattatgaaaaacatatttgtaatccaaacaaaatctaagagtatcagtgtctgtgttgtgctattggtgcattaagttgcttgatttgcatgtttatatggctcatgattgtgcatggtataactagagttactgtgatgtgacattgcagtt

SEQ ID NO: 10 - DNA, 1620 bp, Glycine max
  source 1..1620: mol_type = genomic DNA
atggccgtttcaaccactttctccggtgccaaattggaggctctattgctcaaatgttcttcctcctcttcctcaccaccgccttcaaggtcatcattcaccacttttcccggccaaaacagaagaaccctcattcagagaggggttattcgctgcgacgctcagccctctgatgcatcatctgttgctccaaataatgccaccgctctctccgctcttgagcagctcaagacttctgcagctgatagatatacaaaggaaagaagcagcattatcgccattgggctcagtgtgcacactgcacctgtggaaatgcgtgaaaaacttgccattccagaagcagaatggcctagagctattgcagagctgtgtagtctgaatcatattgaagaagcagctgttctgagtacctgcaatcgaatggagatatatgttcttgccctgtcccaacatcgtggtgtcaaagaagtcatggaatggatgtcaaaaacaagttctgtccctgtttcagagcttagccagcaccggtttttactttacaacaatgatgccacacagcatctttttgaagtatcagcaggtcttgactctcttgttttgggggaaggtcaaatcctttctcaggttaagcaagttgttaaagttggacaaggagttaacggctttgggagaaatatcagtgggctattcaagcatgcaattactgtcgggaaaagggttagaactgagactaatattgcttctggggcagtttctgtgagctcagctgccgttgagttggcctatatgaagttacctgaagcctcacacgataatgccaggatgttggttattggtgctggcaagatgggaaagcttgtgatcaaacatttggtggcaaaaggttgcaaaaagatggtggttgtcaatagaactgaggagagagttgctgcaatacgtgaagaactgaaggatattgagattatctacaaacccctttcagaaatgctcacctgtgctggcgaagcagatttagttttcaccagtactgcatcagaaaacccattattcttgaaagaacatgtcaaggaccttcctcctgcaagtcaagaagttggaggccgtcgctttttcattgatatctctgttccccggaatgtgggttcatgtgtctcagaccttgagtctgtgcgagtttacaatgttgacgaccttaaagaggttgtggctgccaataaagaggatcgcctaagaaaagcaatggaagcacaggcaatcattgctgaagaatctaagcaattcgaagcttggagggactcactggaaactgttcctactattaagaaattgagggcttatgctgaaagaatcaggcttgctgagcttgagaagtgcttaggtaagatgggtgatgatataccaaagaaaacgcggagagctgtggatgaccttagtcggggtatagtgaataagttgcttcatggtccaatgcaacatttaaggtgtgatgggaacgacagccggactcttagtgagacactggagaacatgaatgctttgaataggatgttcaaccttgagacagaaatatctgttttggaggagaagattcgagcaaaggtcgaacaaaaccagaaatga

SEQ ID NO: 11 - protein, 539 aa, Glycine max
MAVSTTFSGAKLEALLLKCSSSSSSPPPSRSSFTTFPGQNRRTLIQRGVIRCDAQPSDASSVAPNNATALSALEQLKTSAADRYTKERSSIIAIGLSVHTAPVEMREKLAIPEAEWPRAIAELCSLNHIEEAAVLSTCNRMEIYVLALSQHRGVKEVMEWMSKTSSVPVSELSQHRFLLYNNDATQHLFEVSAGLDSLVLGEGQILSQVKQVVKVGQGVNGFGRNISGLFKHAITVGKRVRTETNIASGAVSVSSAAVELAYMKLPEASHDNARMLVIGAGKMGKLVIKHLVAKGCKKMVVVNRTEERVAAIREELKDIEIIYKPLSEMLTCAGEADLVFTSTASENPLFLKEHVKDLPPASQEVGGRRFFIDISVPRNVGSCVSDLESVRVYNVDDLKEVVAANKEDRLRKAMEAQAIIAEESKQFEAWRDSLETVPTIKKLRAYAERIRLAELEKCLGKMGDDIPKKTRRAVDDLSRGIVNKLLHGPMQHLRCDGNDSRTLSETLENMNALNRMFNLETEISVLEEKIRAKVEQNQK

SEQ ID NO: 12 - DNA, 1446 bp, Glycine max
  source 1..1446: mol_type = genomic DNA
atgaacgcaacctcatactctgctcttccttctacgttccgcagtctccatcatcggaatttctcagcgttttgttctgatatccaaaatcctggctatgttgattgccattcaaattgtaataagtctacatctcaagcgtctttgtttttgtgttccgactccaacagtagaagaaatggtgtttttggtagaccactttgtgtgaacccctctggcaggagaaacctagttggtccagctttttattctctggagactagtgcttatgacgtggctgctttagaatctccttcccgtgttgcagaagaaaaagttggtgtgctgcttctcaatctaggaggaccagagacattgagtgacgtgcaaccttttctgtttaatctttttgcagatcctgatatcattcgtcttccaaggttgtttcggtttctccagcgaccattggcaaaattgatttctgtacttcgggctcctaaatccaaggaagggtatgctgctattggtggtggctctcctttacgaaaaattacagatgaccaggcacttgcaattaaaatggctttggaagcaaagggcatctcttcaaatgtctacgttgggatgcgatactggtacccatttactgaagaagcaattcagcaaattaagagggacagaataacaaggcttgtggtactacccctttatccccagttttctatatccacaactggatcaagcatccgtgttcttgagcatatattcagggaagatgcctacttgtctaagctccctgtttccattataaactcttggtatcaacgagaaggttatattaagtcaatggctaacttaattcagaaagagctccagagtttttctgaaccaaaagaggtaatgatatttttcagtgcccatggtgtacctgtcagttacgttgaggaagctggggatccataccgagaccaaatggaggagtgcatcttcttgatcatgcaagagttgaaagctagaggaattagtaatgagcacactcttgcttatcagagtcgagtgggtcctgtacagtggctgaaaccatatactgatgaagttctcgttgagcttggccaaaaaggtgtgaagagtcttttagctgttccagtgagttttgtgagtgagcatattgaaacccttgaagaaattgacatggagtacaaggaattggctcttgaatctggcatcaagaattgggcacgtgtacctgcccttggtgttaccccttccttcattacagatttagcagatgcagtaatagaagctctcccatcagcaacagcaatatatgcaccgaccagaacctctgaagatgttgatcatgacccagttagatattttatcaagatgttctttggttcaatcttggcattcatcttgttcttgtcacccaaaatgatcacggcattcaggaatcatgtcatttag

SEQ ID NO: 13 - protein, 481 aa, Glycine max
MNATSYSALPSTFRSLHHRNFSAFCSDIQNPGYVDCHSNCNKSTSQASLFLCSDSNSRRNGVFGRPLCVNPSGRRNLVGPAFYSLETSAYDVAALESPSRVAEEKVGVLLLNLGGPETLSDVQPFLFNLFADPDIIRLPRLFRFLQRPLAKLISVLRAPKSKEGYAAIGGGSPLRKITDDQALAIKMALEAKGISSNVYVGMRYWYPFTEEAIQQIKRDRITRLVVLPLYPQFSISTTGSSIRVLEHIFREDAYLSKLPVSIINSWYQREGYIKSMANLIQKELQSFSEPKEVMIFFSAHGVPVSYVEEAGDPYRDQMEECIFLIMQELKARGISNEHTLAYQSRVGPVQWLKPYTDEVLVELGQKGVKSLLAVPVSFVSEHIETLEEIDMEYKELALESGIKNWARVPALGVTPSFITDLADAVIEALPSATAIYAPTRTSEDVDHDPVRYFIKMFFGSILAFILFLSPKMITAFRNHVI

SEQ ID NO: 14 - DNA, 1943 bp, Glycine max
  source 1..1943: mol_type = genomic DNA
tgattgcggttacatcatgtacggaaaaataattctaatccttgatttaaatttgatcttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgctttgataaaattaattggaaagtataaaatggtagaaaataataaattataattaatttaaataagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttctgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacag

SEQ ID NO: 15 - DNA, 2176 bp, Glycine max
  source 1..2176: mol_type = genomic DNA
aatcaaattatatatgtaaagcaattacagtttatcaaactttatttatggaaataatttattatcacatttattttggtttataaattttaaattaaaatatcacctaaataaaaataatttttaacatgacttattgtcctaaataaattatttccgtaaattaaataaaatgaagtttttttctttcaaagaatctaaatggtcataatgagaattctctaaaaaaatacataatgagaataattatggaatttatttattaataaaaattaatagcattttgatagacaattaataaaattttaaaaataaccatatagaaataataatttttttactatcggttccaattaaaataatgataaaaaataaaatagattattaattgatattgatatgaaatttaaataaagaatataatcatatattttattgatatatgatatgatatagattaattgatattgattttgatatggaatttaaaaataatataataattgtttttatttattaatacgtgtaatcaaataattctcactttttgaatcaatcagtgtacttaaagataatatcagttgaatattttttatccttttacgtgtgctgtgagacattatcatcaattgtgttgtatatgatatatagatatagatatataaatatatagattgagtgatataatatatttaaaatataaattatatatatgttttaatatatttttgcatatatatatatatttgtaaaaactagaagtattttttcatgagataattattatcgagttgaataagtctattatttgtgagagccaaccatatttatatatgtgattaaattttatctttgtgaaattaaaaataataaaaaataccttaaaatcataataatagaaaaacttatatttataatttaccattatacttaaaaaaaattaaataaatattataaatataaatactatcgagtaatggccgcgctagggtttttgagaaaaaatcttcccacgcactcaactgcactgtacggcgtcgttttcacagccgcataatagaagccgcgttccccaacccttcctcacaacattctcggaccctccagcaccgtcacccaaacaaatatccacgcggtagtaggcgcgtgaaacaaactctaatccgaactacgagacgtgagaagcacgcgctttagcgagcgtttcaattgtcgctacgaaagcagagaaggatacaaacggaactagggtaaattagtaagggtaatttcgtaaacagaagaaaagagttgtagctataaataaaccctctaaccctcgtcgcattacttctcttcacacctttgttcactcttcttctcttgcggctagggttttagcgcagcttcttctaggttcgttatctaccaccgttctatggattttattccttctattcgtgtttattctattggtttatgttgcttgcaatatgttttttctgaatctgtcgtcgttgtcttcaattttatccatgtttcagagatcaattttgtttgtgtagtatgtgcttattcttcttcttttcgttcgagttgttaataacggtgctatggtgttttcaaaagtgttttttttattacttttgatttaaagtttttttggtaaggcttttatttgcttgttatattcaaatctttggatccagatcttatataagtttttggttcaagaaagtttttggttactgatgaatagatctattaactgttactttaatcgattcaagctaaagttttttggttactgatgaatagatctattatctgttacttttaatcggttcaagctcaagttttttggttactgatgaatagatctatatacgtcacagtgtgctaaacatgcccttgttttatctcgatcttatgtatgggagtgccataaattttgttatgtctatttttttatctgttggaatcatactgagtttgatgcgttacgattgagcatacctatttttgggcttgttgtatggtgggtatttagatcttaatctttttatgcttatgaaaggttttgtaatgacaaaggtcttaatgttgttaaacttttatttttactttatatggtgtgttgatgtgttatggttttgacaactttttttttttctggatttttgcagatttaaggaag

SEQ ID NO: 16 - RNA, 20 nt, synthetic construct
  source 1..20: mol_type = other RNA
ggctggggaggagatcattg

SEQ ID NO: 17 - RNA, 20 nt, synthetic construct
  source 1..20: mol_type = other RNA
gtttcaatgacttagaagcc

SEQ ID NO: 18 - RNA, 20 nt, synthetic construct
  source 1..20: mol_type = other RNA
gaacagtgtgatgaaaatga

SEQ ID NO: 19 - RNA, 20 nt, synthetic construct
  source 1..20: mol_type = other RNA
gataagatgcgttcaaatag

SEQ ID NO: 20 - RNA, 20 nt, synthetic construct
  source 1..20: mol_type = other RNA
gagaaattaaagtgggaaaa

SEQ ID NO: 21 - RNA, 20 nt, synthetic construct
  source 1..20: mol_type = other RNA
gctttcttccaatcttgaat

SEQ ID NO: 22 - DNA, 8488 bp, Glycine max
  source 1..8488: mol_type = genomic DNA
cgatatcatggaaggtgggacgcggggataaattcaaattttgggaggataattggacagggggagacaattgtttattagagaaatatcaaaggttatacaatatatcggttcagcaaaaccaaataatttagcatatgggagcatacaaggaaacaggatgggaatggaatttcaagtggaggagaccactatttgacaatgagatagatcaggcggttgcatttattcaagaggtggaggggatcagaattcagaatgacattgatgaccagtgggtgtggaaggctgatcttagtggaaggtactcggttaagagtgcttatagtgtgcttagacaggatgtactagaggaggccccagctgaagaatttaaggaattatggaagctcaaagtcccatctaaagtggcaatgtttgcgtggaggttactcaatgataggctgccaaccaaagataatttgaggaggaaacaggtagagctgaatgattatctatgtcctttttgcagcagcgtcgaggagagcgcgagtcatctattttttcactgcagcaaagtacttccgctttggtgggaatccttgtcttgggtgaatatggtggttgtcttcccacaacacccaagacagcactttatccagcacatgcatcaatcgtatgaaggcttgatggcgaaaagatggaaatggtggtggttagtattgacatggtccatttggaagcacagaaatagcattattttctctagcagcaccttcaatgcaaataaaatgatggaagacgcagtgtttttattatggacttggctgagacatttagagaagggttttcaatctcactttaatcaatggtcttcaaatattagagaaggctttcgatgtaatcgaggataggaatctagaccacatacttgtattagtttcattatgtggttcattttctgatcatgattaactacatgcttcatactaaccatatcagaattgtaatctgtcttttgtacgtttagtacctctggtactcaattaattcaatacaatattttggctgataaaaaaaaaaagatatccaataacactttgcttatatgtatgtattatatatgttgcttgctgctgatattaatatatgctggttttatcaacttgctaagaattcttaaatggaatggttgtcactagtagatgctgatatacattggatgcatagagtagagtgttttgttttgttttttttttttgttttttttttagttgcttacatttgcatcgggataacttttaaatatgctctagggcctagaccaatgtccacttgttcagaaagtaatgaaattgaaacatcaaatgcactgatcagttatttatatgcatgattaatgtgcaacaggttctttgcttttatccctctccaacttgcgtatttggcttgagggctattgagtcgaaggagcaaggtgttagggaggaaaaagaaatggctatagcagatgtatgtaaccaaaattggttcctaaacatgttatctctgcaatttgtttttccctcctgtcgtctaatatcacggactattttgctccctagatggagtatatgatagagaagagggcaaagaagaaagtatatgctcgtttcaaggaaattgcacgtctttaaggaaagagacttcctccaaacccatatcccagtccattcaaggagatacaagctgaggagaggaaataagttcgtgaacagttagttgttgtaaattagaaaatttattagttgtgttaggtaactgtgcaggaaaattttaccaaaacttcatgcaagtgtctgtttttccaggccacccatgtaggtttgttagttggtacacaagatctaaattggtaatcaggatttgtatattttctctttttacctttaacattatttgcttcatgaacaaaatatcatagattaattttcaatataatatattttaagttgacttttttgaatgttaaaaaaaaattgaatctgttttagtcaagcagccatatatagcttaagtgttcaagtcatgtggaggtgtcggttaaactgtttgatctaacaaaattttagtcagagacctagtgagtagtgtaggacacgtgttgtacagatgtcgagaaagtgttggtgctacaaatttgtctaatcactgtgcttaacaagtttcatcttattattcagtaactattgtgtttttctttagcagccttttccaatacttatctcttgttctgttcttgtcatccccacttgtactcactattacatcttattctctgtatctttgcatgtagttaagtacatgatcagattttgtggcatccagacacgagatctctcagtatccttttaattattcttgtcatatgcaaagcagcacataacagtttgaagcaattttaatgtgaaatctgtccgatgcatgttaattcccaggttctatcttaaagttgttcttctgacaatgtcatccatttttagcttttggttgttaactttcaaattacaagtaaggtggcagctgttggcgaagaattctgattagttgcgtcctctgatgtataaatactttatggttacatatccgtatttgtatcatacttgtattgaatacccataggcatataataaggctgtctaagccggttatgcattcaatctagacttttattttcaaacttcttatatacaatatttctttaaatagtgaagcaattgttatcataactataatattttatttaaatttggctatgattatgtttttttaagtaaattctataagagattgacgaccaatgatgataggatgatgaagatggcccatcaatataaatatcatctatcctaagaaataaagaaatcaataaaatataaaatatgtaaatataatgttatgtatcacattgcacctaccacttgtacgagttgtatccaacgctatgtaacaaaatgtgtcagatatcagttagataagattcagttcatcaggcagattggataagagtattgctaatatatatcttaaaaaaaggtttcctatttgacagaatgtgggtgtttttgtccatctaggagcttttttcccatgtaaccagacagataattggtatctttaaatttcaaagggtggtccttcacacctaattggagatctaaattgtttatatgtcttcactaataaactgtgtattgctaaatagaagatttacttaagttttttcatgtgaggaagcaagggaggacaaggaaaacggtgaactctcaatgttcagcattttcagaggaacctattatgaagcacttatcttgccgacataggctttctgctctccactaatctttcgaaacttcattttgtcacatgttactaagttgaaaaagaaaatattgttttagaagctaaggatgaaatagtagctaaaggaaagtgatatagaatttgtagtaaaatggaagagtgtaaagctatttactatttgttatttgctttagtacatagaatggataactcaagtgctggttccattaccaaatttcagtggttttggttgaatatggaattcagtggaagtggtgcagaagaggggagggaatgtttggggtacatgatttgaaaaaaggggagaatgattagaagagatagaaaattggcagaccaacattgaaggattcataggaatgttgtgtgttagtaaattggacaaatgttgctcttgatattttcagcatatcattcatcttagaagcccaactgttcttttttatatatattctctgttgccttcctttccttcaaaacacggcttttgaattttgaaccattaactgagttggaatagtggtaaaatcatgtagatttttcttttcgttttgtggggatatgaatacgaaatatccttacaaccagacaaagctgtggctcagaagaaagagaaacatgcctcactgctagaatcactagaactcacatcttgtatacaaactgttataccaaactgagaattttacaatttccaaagatgcaattctcaaccattaaccaatttcatcttaaacactaacagttcttcagtcttgaactttccatgctgaaaatttgttgcctatttcttttataggcagtggcttcttatgagagtcacactctctgaacttcttacgtaatacttgacttttcattttgtgctttataattgttatctcatgcattgcacttttaagataaggttgttgtgcattgtttggttgagtgcaacgatttactgattaccatagctacttgtggatcttatattttcaaaagtgtatgcttggtcattaatcaaccaagcatgataagccccatcatctaccaccccttctataccatatacgcattttgcttatctacattgctactgttacactcaaacaagtctaacagataatacgttaagttaatgaacatttttagtaatattattataaggattggccaatgtaattgtgaagagagaagcatgctttaagctacccaacaaaatggataagagtttcagttgatatggttctcttgtttcttttccaataaaaaaccaactttaaaatataaaatttactgtaacaaaggaacaaagagttttcacttaatccatgaatgagaaaggatggtcacaaaatatgttaggttaatatggaatgagggcactgtgcaaactacacaaataatatcaattccaccaccatcacacatttacgttcttccaaggaagagataagataatggagcctccacgtgtcacctccacatggtacctaacaataaggctaccattcaaaattttcctcactcgtgtggcctatatgctgtaatgtcatcacttattcaatccaacggttgtaacttttcggcaaccaatcctctccatttcacaccattggattagtactacacaaatcacactattatatatagcaagtttgagcagaagcttggatatctggcagcagaaaaacaagtagttgagaactaagaagaaggaaatggcttcctcaatgatctcctccccagctgttaccaccgtcaaccgtgccggtgccggcatggttgctccattcaccggcctcaaatccatggctggcttccccacgaggaagaccaacaatgacattacctccattgctagcaacggtggaagagtacaatgcatgcaggtaagacaactccacacatatatacacacaagaggcaccaaaaagtttaaaattcatcttacacatttatatatgctccaaaatgttacttaatttaacatgttagtcataggttacttaaattaacatgttagtcataggaaaatgtgcacatgtgcatgtttgttaactaacttttcatgcaaattactaggtgtggccaccaattggcaagaagaagttcgagactctttcctacttgccagacctcgatgatgcccaattggcaaaggaagtcgaataccttctaaggaaaggatggattccttgcttggaattcgagttggaggtcaatttcttgcaaccccttttggtttaattatagtttcttagtcttagtttgtttcaacttattttgaagaaataatcgcttctcttgctggcgtgctaaaagtacttttggaaacaaaaaaaaaaaaaaaaaaaagagaagtggttgttttccaaaatttaagtttatctaatggatttttctatctcagacttatcaatattgatcaaatctgtccctattattacaaaacaagcacttggttttgagttttgatggttgatatgtttgttgtgtttgtatatagcacggtttcgtgtaccgtgagcacaacaggtcacctggatactatgatggacgctactggaccatgtggaagctgcctatgtttggctgcactgatgcttctcaggtgttgaaggagcttcaagaggctaagactgcataccccaatggcttcatccgtatcattggatttgacaacgttcgccaagtgcagtgcatcagcttcatcgcctacaagcccccaggcttctaagtcattgaaaaatttgcaaagcatctgtagccaccccactttgtttgttgtacttaaactacattcccatttgtttttgctttatgagatttcatcatcctgtatttttggtttctgttttcggacttcaatggaaattaatggatgagaactaatgaataagctattgtgttgtgttgctttgtttccaaataacttcaagaacccattgtccttgcatttccatcttgtgggttgaaattagtctcttctaaatttaagttaattgtgtcactaaatgatggttaacaaaggaattctttctgcgttattcaaagtacacagcatagtttagttgagcttggccttactttgtcacaaatctgatgtatgaatgttctttatactctggttttccaagaaatgaagttcaagagatagtttgtaaacatgttgctactgaactttcaaaaccaaacatgagccccataattcttggaaacacagatattttgtatggtatatagcaacaaaatctactaaggaactgctaccaaaccgttttgttatttatgtgcaaagaaagcgatatcaaattcaaatttttgttggtttcagaaacttttctttccaagcaataaccatcaactgcaactaaaccgttttgtttgttccatttctcatctcctcactgcaattttcaaattatgatttggataacacataaaaacacaatcagatttggtcatgctctggttctaccaaatacttttttttttctagaattgcctgaatctgatccttagctgacaattaatctcaagatgattctattgtttgtaacttgatagttatggattagcaactgatagataaatcatggttctaagttgtgagtagttataggtaacttatctcgacaccgttgttagtaggttgcattaatatttagattggaccctttcgatataacaatgatacaataagataaagaaatagataagtttcacctcctttactcccatgaccacatcatgacatattcaaagtcaatggtgaatacacaaatcaaaggattttttttccaaatatttatttgtggatgtgaatatactcatcctagaagcaagtttctttaaatatactttgtttaataatctttttgtgaacatcaattctttcttaatccaactgttatatcgaaaattctcactcagtagatcaaatatgcaaacttttaatcaatattatattaatccaagtataacagactcaaattctttaaagaaggtcttgatctcaaattttatgaataaaaaatattgttaggaggagagacatcacactaaacataatcaaccaagtcatgacaaagtcatacaattcaccaaacaattgatattttaccaatcaacttcattttaatgagtattatacttttttttaaaatacaagtaatatgataatattacataatataatagttaaggatccgtatgggtggtttttaattggaagtttaaaatttttaaaaataacaatcaatttttagttttagttacaaaagtttttaaaactagtctttaaagtttggttgctttctaatttttagttttaaaatactcattttaaaagttttcaatcctattaaaatgagtgtgtttttatggtgatgatgacgttaacaacaataatgatagtatcaatggtggtgatagtaatgttagtgacttattggtgacaataagtgccaaatggtactgctagcttatagtggtggctagtgatgattgtgctggtagtagtagtagtagtgatcgtggtagcggtgttaatggtgattgcaataacaatagtgtgacaacaatagtgtcaacagtgatggtagcaatagtgatgatggtgacaatgttgtaagtgaagacaatagtgatggtagtaatggtgatggtaatatgttggcagtcaagacaataatagtgctcgtggtggtgacaatggtgatgacaacaacaatagtatcggcagaggtggtgatcgtgctaatggtggttacggcgacgatgttggtgatgattgtagggaaggtgcagtgtcggtggtggtggtggttgtgacagaagtgctggtgctagcgatggtggtgagaagtgttattatcaaatgtaaagtgcctgtacttttttaaaattaaaaataatatttttcttgattttaaaaatgaaggtaaaaatattttgaaattgaattaactattttagttttattttttgtcaaatacaatttgttttaaaaatttacagttgaaactaaaaaataaaaaaaagtcacacacatcctgacccggaccctaagtctttagaattaattagtattctataaaaaaaatttagcatgctttaagtatttactcaattattcttatagtgaatagttccctttcgt

SEQ ID NO: 23 - protein, 178 aa, Glycine max
MASSMISSPAVTTVNRAGAGMVAPFTGLKSMAGFPTRKTNNDITSIASNGGRVQCMQVWPPIGKKKFETLSYLPDLDDAQLAKEVEYLLRKGWIPCLEFELEHGFVYREHNRSPGYYDGRYWTMWKLPMFGCTDASQVLKELQEAKTAYPNGFIRIIGFDNVRQVQCISFIAYKPPGF

SEQ ID NO: 24 - DNA, 8506 bp, Glycine max
  source 1..8506: mol_type = genomic DNA
tgaaaaaaaatgagaagtgccaatgaagtgtttgtattataacagatgtccctatatcatttttcttttatagatgaggaattaaaaatatgattttatttaaggaacattctatttttctaggaacattttcttatttacaggaataaaaatatactattcaaacttttttttcctgcagggtgaaaatttgagtattattatctttttgactgtgtttacaagagcgccacctgcaacatttcctagagctgaaaaaaattagaatatgctctgctcttaagcatgaatatttatctgaatttctaaataagcgcagaaactgcttagttaggctttatgttgacagctaaaatattgaaaatgttgagttcagtatgattgttgtaccattgttctcaataatattgatcatgttcatgactgatgtttattcaattgattggaggctatgttagcctttagtgctgctcaagtatttttatgtgacagatctgtcataatgaatatggtatttcaaatgcatgtggaatatggtatttttctctagttataatgaatatttctcatctgtagtctgttatacaaatgtttttatataatagatctgtcacatttttttcaaatgcatttggaatatgtatttttctctagttataatttctcatttagctacttgctgagtggattcaagtataatctgacactctgagttcctttccatagaaaaagtggaaactgaaaagaggtgtactttctctcattctctttcttcattttgtcttgccctttctgggtgtggactgagttcttaaatatagaactactcatgtgaaagattatatgtctctgctctgtttctgatatgtgatatctgataactctttgcttatatgtatatatattatatatgctggttttatcaacttggtaagaattcttaaaatgaaatgtttgttactaatagacaatgttatcaatggtggatggtggaatgtagcggaaagtcaaaattccaccacatgaacacagaattgtggcctatggtgctgtgttatgagtcacacattgagtagagtaaactacttggtgtggtacatagtccttgaggttcttcacctaatgaaccagtcttttgggttgagctcttctcttgtgcttaatcctaacaattaatgcttttggaatgactgattcatattggatgcatagaattagagtagggtattttttttccaagttgcctacttttaaatatgccctagatcatgtccacttagttcatatggatgattaattttcagtaggttctgtgcttttgatccccctccatctctcgtatttggattgagggttcttgagttaaaggagcaaggtgttagtaaggaacaagcaatggctatggcagatgtagtaacgtgaactggttcctgaacatgttagctctgcaatttgtttttcccttgtgttctcttatcctctaataatccactttggcttttttgcttcctagatggagagtatctgacagagaagagggcaaaaaagaaagcatatacctgtttgaggaaaaaacccatatcctagtcctatcaaggaaatacaagctgaggagaggaaattgtgaagcaatagaaagctgagtcagcagagagattcagagagggggtgattggtgagatgtagaggggaaaagttacactactttgaaagtttggctcttgaacaaatgatctttgacaataacatttactgttcagccactgaaaagttatttgttgtaaataagaaaatctatttgttgtgttagataactgtagaggaaaattttaccaaaatcttatgcaagttttctgtttttccaggccacccatgtaggtttgttagttggtacaaaagaactaaatagataatcaagatttgtatatttactctttttatttttatgattatgagcctcatgaccaaaatattatgcgattaattttcaatataatatattttaacttgactttttgaatgttaaaaaattgaatctgttttataatatcaatgttcttgaagtatccagccatatagatagcttaagtgttcaagtcatgtggatgtgtcaagtaaactgtttgaactaacaaaacttttgttagacttagtgagtagtgtaggacacatgttgtacagatgtcgagaaagtgttggtgctaaaaatttgtctaatcactgtgcttaacaagtttcatcttatcattcagtaactattgtgtttttctttagcagccttttccactacttttatctcttgttctgttcttgtcatcctcacttgtactcagggtcacttgttgcatcttattctcttgatctttgtatgtagttaagtacattatcagattttgtggcatgcagacacgagatctctcaccatccttttaattattttcatcataagcagcacataacactgtttgaagcaattttaatgtgagctcctttggatggatgctaattcccaggttctatcttaaagttgttcttctgacaatgtcatccatttttagctttaggttgttaaccaaaatagaagcaaggtggcagctgttgctgaagaactctgattatttgtgtcctctgatgtacagatactttatggtcacatatccatatttgtatcgtacttgtaatgaatacccataggcgtataataaggctgcctaagtcgggtatgcattaaatctagactgttttattgaaacttcttacatacactatttctttaaatagtgaaacaattgttatcattaagttttttaataaattcgataagagattgaacaccaatgatgttaggatgatggagatgacccatcaatagatatcatctatattaagaaataaagaaattaaaatataattttatgtatcacattgcacctaatacttgtatgagttgtatccaaagccatgaaacaaaatgtgtcagatatcagttagataagattcagttcagcaggcaggttggataagagtattgcttatgtatatcttaaaagaaagtttcctatttgaccgaatgtgggtgtttttgtccacctaggagctttttaccatgtaaccagacagataattggtatcttaaaatttcaaagggtggtccttcacacctaatttggagatctaatttgttgatatgtcttcactaatagactgtattgcaaaattagtaaattgaagatgacattactcaagtttttcatgtgaggaaggaagggagcacaaggaaaacggtgaaacctcactgatcagcattttcagaggaacctattatgaagcactcaacttgctgacataggctttatgatctgcactaatcttttgaaacttcattttgtcacatgttactaagttgaaaaagaaaatattgttttaggagctaaagatgaataagtagctaaaggaaagttatatagaatttgtagtaaaagggaagagtgtaaacatatttgcttcagtacacagagtggatgactcaagtgctggttccattaccaaatttcagtggttttggttgaatatgtaagtcagtggaagtggtgctgaagattggagggaatgtttggagtacatgattagaaaaggggagaatgattaaaagaaatagaaaattggctgactaatattggggattcataggaatgttgtgtgttagtaaattggaaaaatgttactccattgatattttcagcataccattcatctaagaagcataactgttcctttttatatattttctgttaccttcctttccttcaaaacatggcttttgaattttgaaccattaactgagttggaatagtggtaaaatcatgtagatttttgttttcgttttgtggggatatgaatgagaaaaatccttacaaccagacaaagctgtggctcagaagaaagagaaacatgcctcactgctagaatcactggaactaacatcttgtaaacaaactgttataccaaactttcctaaggtgcaattctcaaccattaaccaatttcatctcaaacaacactatcagttcttcagtcatgaacttcccatactgaaaatttgtcacctatttcttttataggcagtggcttcttatgagagtcacactctctgaacttcttatgtaaaacttgacttttcattttgtgctttataattgttatctcatgcattgcccttttaagataaggttgttgtgcattgtttggttgagtacaacgatttactgatgaccataactatttgtggatcttatattttcaaaagtgtatgcttggtcattaatcaaccaagcttgataagccccatcacctaccatccccttctatgccatatacacattttgcttatctacattgctactgttactctcaaacaagtctaacaaataataagtgaagttaatgaacatttttagtaatattattataaggattggccaatgtaatggtgaacagagaagcatgctttcagctacccaacaaactggataagagtgtcagttgatatggttctcttgttttttttttttttttttccaatgcaaaatcacctttaaaatataaaagatcactgtaacaaagggacaaagggttttcacttaacccatgcatgagatatgatggtcacaaaatatgttaggtttaatatggaatgagggcacagtacaaaccactcacaaataatatcaaactccaccaccatcacacattttacgttcttccaaggaagagataagataatgaagcctcctccacgtgtcacttccacatggtacctaacgataaggctaccattcaaaattttcctcactcgtgtggccaatatgctgtaatgtcatcacttattcaatccaacggttgtaacttctcagcaaccaatcccctccatttcacaccatcggattagtactacacaaatcacactattatatatagtaagtttgagcagaagcttggatatctggcagcagaagaacaagtagttgagaactaagaaggagaagcaaatggcttcctcaatgatctcttccccagctgttaccactgtcaaccgtgccggtgccggcatggttgctccattcactggcctcaagtccatggctggcttccccaccaggaagaccaacaatgacattacctccattgctagcaacggtggaagagtgcaatgcatgcaggtatgacaactccacacatataaatacacaagaggcaccaaaatgattataattcatgttacatatttaggcatgtacctaaatgttacttaaataaacatgttagtcataggttgcttaaattttagtcataggaaaatttgcacatgtgctagttaatctgttaacccctttactcaattttcatgcaaattaattactaggtgtggccaccagttggcaagaagaagtttgagactctttcctacctgccagaccttgatgatgcacaattggcaaaggaagtagaataccttcttaggaagggatggattccttgcttggaattcgagttggaggtcaatttcttgcaaacacttttggtttaatcatagattttcttattcttagtttgtttcaacttattttcaagaaataatcgcttctctagctagtgtgctaaaagtacttttggaaacaaaacaaaaaatggttgtgttccaaaatttaaatttatctaacagatttttctatctgagagttatcaatatgtatcaaatctgttcctatctctgaccaaattttattcttccaaaacaagcacttggttttgagttttgatggttgatctgcttgtttgcagcacggtttcgtgtaccgtgagcacaacaggtcaccaggatactacgatggacgctactggaccatgtggaagctgcctatgtttggttgcactgatgcttctcaggtgttgaaggagcttcaagaggctaagactgcataccccaacggcttcatccgtatcatcggattcgacaacgttcgccaagtgcagtgcatcagcttcatcgcctacaagcccccaggcttctaagtgtgaaaaatttgcaaagcatttgtagccgccaccccactttgtttgtttgtacttaaactacattcccatttgtttttgctttttgagatttcatcatcctgtatttttggtttctgttttcggacttcaatggaaattaatggatgagaactaatgaataagctattgtgttgtgttgctttgtttcaaaataacttcaataacccatcgtccttgcatttctctctagtgggctgaaattaatcttttctaaatttaagttaattaattgtgtctctaaatgatggttaacagaggaactgtgttattcaagtactctgcatagtttagctgagcttggccttactttgtcacaactcacaaatctgatgtatgaatgttctttatactttggtcttccaagaaatgaagttcatgacatatatagtttgtaaacatgttgctaggaagcttctgaactttcaaaaccaaatatgaacttgataattcttggaaacacagatattttgtatagtacatagcaacaaaaaaccactaaggaactgctaccaaatcgttttgttatttatgtgcaaagaaagcgatgtcaaattcaaatttttattggtttcagaatcttttctttccaagcaataaccaccaactgcacgaaaccgttttgtttgttccatttctcactctcctcactgcaatattcaaattctgatttggaaaacagcacataaaaacacaagatttggtcatgctctggttctaccaaatactacatttgtttctagaattgccagaatctgatccttagctgacaattaatctctagaagattcttttgcttgtaacttggtagttatggattagcaactgataaataaatcatttagtgggctctatgttgtgagtagtgataggtaacttatctcgacaccttttagtagattgcattaatatttagactggacccttttgatagctacatttgtgaacaagaatacaatacgataaagaaatagataagaagtttcacctcctatactcccatgaccacatcatgacatattcaatgccaatcatgaatatacaaaccaataggatttgttttcttccaaatatttatttgtggacgtgaatctactcaccctagaagcaagtttcgttaaatttactttgtttaataattttttgtgaacattcattcttttttaattcaactgttatataaaaatatctcactaagtagatcaaatatgcaaacttttaatcaacattatgttgatccgaggataattaaattcaaattccttaagaaaggtcttaagttcaaattttgtgaatggaaaatataattaaaaaaaaagacataactaaagtagctagtcaagtcatgataaagtcttacaattcatgaaataactaatattatatcaatcaacttcattttaatggatagtagtattattcttttaaaatacaagtaaatgttaactataagataatctttatggtaatatattacaaaatataatagttaagggtctgtttgggtggttgttagttgtaagtttcatttttaattttaaaccagtctttaaaatttggttagttttctaattcttagttttaagataaagccatttaaaaaaatttcaatccttttaaaattaatttaaaaggtgataatgtcaacataggttatggttctagtgttagtagaggcgaccagaaatgtcattgtcaaatgtagagtgtgtactttttttaaattaaaatcaaaattacaaatgttttttttctttattttgaaaataaagataaaaaagtgttttgagatagtcaatcatttcaacttcattttctgcccaaagcttttgattttgaaaattacatttaaaaataaaattaaaaaactcacaattacctgaccaaaccttaagtcttttaaattaattaatattctataaagaaatagtatttttaaaatatttatataatttaagtatttacttatttttagagtgaatagatccctttcgtttgtttcattcatcatacagaatcaataatacactggaatattcgcttatatatatatatatatatatatatatatatatatatatatatatatatatatatatataaaatgtacatggaaaaaataattttccattagtgtattaaaatttgaagagagacagacaaatatatccttatttccatagagcctgcaaaaacctaactatccagtcaatattttatatttccgtagtttttctgacaaaccaatttctttaataaagttttcaaataagttatataacatatcataatattcaaggttttccaagagaaatttaaaaattgttattttgacaagataagtgctacataattgtgtggtctatagcgactaatgagtcaaggcatcgtgact

SEQ ID NO: 25 - protein, 178 aa, Glycine max
MASSMISSPAVTTVNRAGAGMVAPFTGLKSMAGFPTRKTNNDITSIASNGGRVQCMQVWPPVGKKKFETLSYLPDLDDAQLAKEVEYLLRKGWIPCLEFELEHGFVYREHNRSPGYYDGRYWTMWKLPMFGCTDASQVLKELQEAKTAYPNGFIRIIGFDNVRQVQCISFIAYKPPGF

SEQ ID NO: 26 - DNA, 8635 bp, Glycine max
  source 1..8635: mol_type = genomic DNA
aaaaaagagaaaacaaaacttaaaaatgagattttaaaatatcagaaagagaaaaacttctagcttttaattttttatatttatttttcttttgttcttgataattttttaattttttatttaagataaaattaaaaggagaaaaaaattcaatggacaacacatgcgctctgaataatgcacgttcagagatggtgacaaatgatctaccaaaaaaaaaagcagcattgcaacctacagaaaagagatggtgacatagggtggtgagtcaccggcatttatcataatgctctagacccaccagcatcaaccaattagatatgcacgatttttaggggaaaaaaaaatctaagtgtccacctcattttgggtgaattgacgtagaaggaggagcaagattagaaaaaaaaaaaagaagtaaaattaaagaacatgataaataaactttatgtaatgaaaaatttaacaaaaagaacatttaataatatttttatcctcaaatttgtgtcaatactattttagatccccgaaatttaaatttgttttttttttctcaagtctataagttatcttttgagtttcttgtgattgatttttttcccgtaaagatgtatagtttgtggcatttttttattacaagaaaaactaaaaaaaataaaattgagaaactaaaaaaataaattaagatgtttgtctctagtgtagcttttaaaatatattgtaatgctgtataacatagaatttataaaaaaaaattagtaaatttattttctatttaacgagtttttctgttgattttataattttaataaatgttaatcaataatttattaaaaattatcttattacaaataactcttaatgataaggatgagataaaggcccaacacctatcaaaaattgccgcagggtagttgaaacgtgcttcttatgccgttgtgactttgtggtccaaaattaaccgtacaatctgcaattattgcaatgctccgtctttttttagcgtctgtatgtcaccacttggcacgggcacttaccgataaaatataaaatgtataaacaaaagtaattacaatttgaatataatttaaaacaaaaaaaataaaaagtatattttagcatttatatatataattattatgttgaaatatcttttatctttaatctttattctttataccgaatctaaaaaaattcataagtctataatttaagacaatttatgaaattgagatttgattattaaagatagaataaaataactcaatcattaattgattttttatttaagtattcttcaacaattattgtaagatctttacggctattcattaccaatacgaataatgatattatgatttttgtcgactggaatttttttgttcttcaataagacctttgtgactatttagatggggagaaaaaaaattgtatgacgattcggtatattgaaaaccataacctcataatgtgacaatattgacatctgatcatttaagttcatatcattttatttagttgtctaacagactcacctaacttaacaacctaaaatatactcaataatgataaatagttaatataatgatcttacaatattatgtaattttgtcatttcgcaactctgttcatgttagatatgtataacattatatgttgtagaacaatttaacacttattgaagaaaataaaacaaaacgtgaatggttttagatgttgcacactttatttatcatatctagtgtggtattcaccattgtagttagatttgataatcttaattttcttttgtaattgaagttcaagtttaacttttaaagatttgaaaatgtatagagactgaggcatctcatcaattaaagaaagagtagtcatctatgaacataataaaatacatttgtctattccatgaatccataagaaagataccacgaatatttgtaagtattagttctaagacattcttaaatctttcaacacttaaattccttttatttgtttgttcttcctttgtgcactcaatacagacttaaacgtttgacaaataaaaaggcccaaaaatttcctccgacacaagctttttgaattctctatatagagatgtggcctaagcgcccatgctataaagtgacaaaattctcatttaatttatgtttcataccatgtgaatcctttgtaacatttctttataggagcacttaatatcaattaaagacacataaccaacatcacttgaattacatgagagactaactttttatttccaaatgaacaagagtaactggatttgtccaaaatagaaatagaaattgaatttcacctaatagatgatgtaacacacacacacacactaaaataagggtctaaagaaatttttcaagcaaacacttggtttgaaaaggatgacaaaaaatactaagagaatgcttaataaaaccgttaagagatgtagaaacacttaatttttactcattcactcaaatagagttatatctagttcttctttacacaatcgtaaagggtttcactaatcaaaacttgattacaaagcaaatattatgtcttgctggctatacaagtattattctagtcacttctacactatcttagactccccctaaacttaagaaaaacctaagaatggtttaacattaagtcagtctaggctttcacaagtagtagtttgaataaatacaagtattcaatagcactcaatgagttggataagtaataaagcaaaaataaaagataactttgtttagcaaaggataaacaataagagctaataaattgtctagtgatttctgtagagttttgcttcagagttattcacgttttctcgtaattttttgaagtcgtgcttttgggtagagtcttccttttaatagatttcaatgaaggattcattatggtattaatgttaagtctttgaaatgattgttgtctcacattggagataagattatgtgacatgctttgatttgaaaggcaaagaataaaaatacataatactgcatgtgctcctagaggaatattttgtgtagactttctattatcttttattttttcattttttaaaattcaaatgttaacaattcaaataaagagagaaactaaaatttcataaaagagaatacgtgtattaattttatttttggttgacatgactttttcatactaagttggacaccaattgtgtgtgagacttataccatcttgtgagtgttgaacattcttagtgtataacactgataatataaaggggtagacacttttggtcttatagcgtgtgtgatgctgattaataattaacaaaatattttcttttttgagtgtggatgatatatgtgaataacacttaagtcctttaataactttgctcacgtccacttgtcataaacttattaatatatcaaaaatttgtattttttgttaaacatcaaaataggtataaatggtcgtccaaacctaacaatgagcatttaatgttaatatgaattgaagatcatattaacatgtattacatcggacctgagttctttgttgttcctatctcagttgtccccatgctgtttcttgaaaatcacacaattttaaattaaatcataaaattaaatattaacacaaactaaatagataacatcgaatcagctaaaaaaaagacaacaaaaaaaccaatttagtattacatctttcaaacaagacatatgttgcctaaacaattttttggtacatttcttaattgaaaattttacactattctgaataatttattagatacataatatctatgtgcaaatataaaatccatacaacttttcagtgttataggtgaactcatgtatcacgtgttctaataagtctctatatattcagatttaagtgataagagtggaccccgaaaagaaactgtttttttatatatataaacaatactatgctataataaattttatatatatatatatatatatatatatatatatatatatatatatatatatatatatatatattaagaagaagaaaaaaagcgcaagagcatcgatcaaatgaacgaataatgctcaattttcttttattaaaagtataatttaatttatacatattgtatgatgtaatattggtataaaatatttaacatatttgatgataaatatttactaaaaataaaaatttgtatgattatctttatcaacacaattttttttccattcataaaatttgaatgtgactttacttaagagattcaatataattaactatcatcaaacagcttcctggtatatgaacatgcaggctttttctttgtaatctgaggggtatttcttatgcctcatatgaatattattctctctttgccgtttcagaaaaaaaaaaaaaaacttaagaatatacatgcatatatatatatatatatatatatatatacatttatcttaaatatatattttaaagtaattattataaaaataaattattttataataaagaaccatctatgatcaaataaatgatatatataacttgaatttgcctgctgttagataatttaacaactaattaactgtaagtaatctcagttccttaccgctagaaaagttaacatttaatgttctgacacattcagaatttgttatgtacgtcctttacattacacccaatactttatctttcaaaatgagtttctcacgttaaaaaaaaaaaaaaaaaagttgcatccaacagactttaatttgtacaaaatgcgttttgattattaaattctaaaaaattaacctacctgattgttttttttaaaaaaaaaatatgttcaaccaaattctattgaagattaaaaaaaaagtagacaaaaaacataaaagaaaataacattctggactagtaaaatgaattattggaccgtaaaaattagcttattacagttcagaaatattttatcctctatgcagtaggcagaggtattgtctatttaaagaagcaaatcctcgatgaggaaagtgcacaagtgagtttgttgatcgagataactagaaacagtgtgatgaaaatgaaggtccttgttttcttcgttgctacaattttggtagcatggcaatgccatgcgtacgatatgttccctctccgaatgaacactggctatggtgcccgtactccggaggtgaaatgcgcaagttggaggcttgctgtggaagcacacaacatctttggctttgagaccattcctgaagagtgcgttgaagcaacaaaggaatacatccatggcgaacaatatagatcagactccaaaacagttaaccaacaagcttacttttatgccagagacctcgaagtccatcccaaggacacatttgtgttcagtatagatggcaccgtactctctaatattccatactacaaaaaacatggatatgggtattatgctcttagttaattttcttctaatactctctttttccgtttttgctattactaggttactcccttatatgcgcatcatttattttcaactatttttggtattgatcgagtttcggtgtttggcccgactaacaacattaaggtttctcatcacatgcatgtagatttgaaaccttcattatatatgacattttatgattagatataatataaagaaaaagtttcattgtgagtgcataaatatatatatatacagacacaggcacatacatgctttcaggatcgtgttcattgttcttaatgcaatattttatgtgatagggtggagaaatttaactcgaccttgtatgatgaatgggttaacaagggcaatgcaccggcactgcccgagactcttaagaattacaacaagctggtgtctcttggcttcaagattatattcttgtcaggaagaacactggacaaacaggccgtaacagaagcaaacttaaagaaggctggctaccacacatgggaaaaattaattctcaagtcagttttcttttttctctatactatattctcaagtcaattaattctcaacgttgcaccaaatctgccaggaaggaacagtttatagataatcagaaattatataattattttaagtacatggtgcgatatgagtttgaaatactagtagctaattggatctatttttggttggtgcagggatccacaagatccttccactccaaatgcagtttcatacaaaacagcagcgagagagaaactgataaggcagggatacaacattgttgggatcattggagaccaatggagcgatctcctcggaggccacagaggcgaaagcaggacctttaagcttcctaatcccctgtactacattcagtagttccttcacctctcaacaatctagcttgctcctatctatctatatgtaataagatgcgttcaaataggggcactagcttcactttatgttgtttcagctgtgtgtcaacaagtcatgtaagagacttcactatgttgttttctctttcgtggctttaataaattatgcgcccttgttccatactttgatcttttcttttcttttctttttctttttaagtcaaagtaattcattattatcaaactaatgaaaaatactaaccaatatcctaagagaattggttaaagaatttaaaataataatatttttattaagagacacaaaattatgttatttataaattttttttattttcatataacttacacaattaatatttttgtcttttagttttataacaaatgtcttaaaaacactaattaaaaagattctttaaaaaatagtcttaagaaaatctgaggaacttagctaggttttacaaactaatataaaatgtgaattaaatatattttttccatttaataatttagtattttttttcatccattacattattttgaataataaaaaatattataaagtgttttaaggatgaaaaataaaagaatatattttaaataaaataaaaaagaaatttgcaggaaaaaaatatctaaaagataaagattttataaaatcgagatcgggaaagaagtgcacaaaatcgacgtcgtgaaagaagtgcacaaaatcgacgtcgtctattacaccatataattacactgtgaaccgacagttggtattaacacaatggcccaatagagtagaccgagaaataattatctgacaaatatactgttcactaaaaacaaaaaaatgatgagaaagcctgaaaattacactatatatatatatatatatatatatatatatatatatatatatatatatatatatatatgaaatataataactttatactatcatttaattaaaaataattatttaaattattttaaaataattattttaaaaatcaataaatttatcatacataataaatatttaatataaatattgattaaatgattatataaaaaatttatactatctttgtataatccttttttcatataaaaatattagtgaatggtgttttgagtttcaaaattttataaaatttcgttttaatttctaatttataaatattactcttttaatataccctttaatttctaattttaaaaatattactcctaatacagagaaattttagttttaaaattccaaaatcgagtagttttgatttctatattttttgaaatatacatatttggtccctgtataagatagaaaaacattagttttatatattacgtttggatattttttataaaaaaaatcaaccatgaaaaatattcattttaaaagtcagaactcagaatcaaaattcaaagtttagaaactttttaagtaaaaacattgttaacccattaaaatgttgtcatcacatacatgtaactaatgctaaatttaattacgtttatcttttattatatataaacatcttaaaaatctattaaaaaatactaggtacaatcacactcatttaactatatattttttattgatgatgctatttttttggtattttggggttgctgaagaacttgacggtttttgtctacaaccctcctctaacctaaaaaaataaattggttggcgagagtattgcagcaaacatttgctggagtatgtttgacgcttaagaagcttttgaattaaaatataaatctcacaaaaatatataaatttatcctttaatattatatctcttctctttatccaagacaccaaaagtctcttgcaactttagcactgttagcgactttctgacaattagtcacgcgcactccctcattgtgtctgtactctggagtcaaactcatccattttggaagcgaaataatgcttttgggtgaaaaaattatctctcatgctttagtccgtgtactgtttggactttggagtgtatgattttatattaactttatattactttttctgcttttgggtgtggaaaaaattatctctaatgctttgtttgtttcaatgcacggaatgatttagagagatggaaaataacttcatattttgtccctgtaccgtttggactttggagtatattgagaaagaaaggaataaaagtatcagtttgtggttccatattaactttatatatcgtctaatttagaacgaaaaggagtattttctatacaaagataaaaatattattatttacaatatatctcatgttttactattattattaaaatatag

SEQ ID NO: 27 - protein, 267 aa, Glycine max
MKMKVLVFFVATILVAWQCHAYDMFPLRMNTGYGARTPEVKCASWRLAVEAHNIFGFETIPEECVEATKEYIHGEQYRSDSKTVNQQAYFYARDLEVHPKDTFVFSIDGTVLSNIPYYKKHGYGVEKFNSTLYDEWVNKGNAPALPETLKNYNKLVSLGFKIIFLSGRTLDKQAVTEANLKKAGYHTWEKLILKDPQDPSTPNAVSYKTAAREKLIRQGYNNLACSYLSICNKMRSNRGTSFTLCCFSCVSTSHVRDFTMLFSLSWL

SEQ ID NO: 28 - DNA, 11535 bp, Glycine max
  source 1..11535: mol_type = genomic DNA
ttttaaattttaaattgaatcaaaataaatcaacatatttgagtctctttgggttgataagtttatctaaaaagacatctaaaataaatgaaataatttttttcatcagttcaaattaacttgtagaaattaactatcttacctttttaaaaaaaattactaaaaaaatcttatacaaattagcttatgcataacattagaaaaaatcatttgagattttttcttaaaaattcttatgcataaatttatccaaacaaatcattaatcatatacgataatttataattaaatcataatataaaaaattacacactaaatatataaactatttattcatatatatatatatgtatatatatatatatatgtatagacacacgcacgcgcacacatgttttcagcatcttgttcattgttcttaatgtaattacaagttgctgtctcttggcttcaagattgtgtggtccggaagattacttgacaaaaatgccgtaacagaagctaacttaaaaagggatggctaccacacacacatgggaaaagttaattctctagtatgtttccttttttctctctatacatataatttctttatcgttcatatataacaactataactaactattaaacataaaattaatttagttagttattagaagttatttgagtaagttggttggttactcaagttatagttattagaagttatttaagtaagttaatgttggttactcaagttagttattttctatctttatataaatacatcaactttgtaatactttgatcaatgaatgaatgaattgaatgaatcctataactacttttcatctatctttcatttctttcattcctaatatggtatcagaagtttttgcttccgtgtctcttacttcctattaaagctatttatggttctacccggtggtgtagatagtgctgttagcccttgctttgtggcgtcgagccctgactcgagggggcgtgttgcgagtcccacatcgggtggttcacattgatgattaagttcttatatgactgggtaggttatcccctttatgaatcattttttaagggaacctttcggatacttggttggttctttttttttctctctcatggccatagatgtcattgatgattaagttcttatataactgggtagaccatccccttatgaatcatttttttaaggggacctttcggatgcctcttggttctttttttttctctcatggtcatggatgtcattgatgattaagtttttatataactgggtaggccacccccttatgaatcattttttaagggaacctttcgaatgcctggttggttctttttttttttttctctaatggccatgactgccaatgatgattctaaccccttcattcttcattcttctgaaaatcttgacattgtcttagtttctcatcctttcatcgtgttttcggcaaaggcttagttgctgatgtggtgttgtggatgaattggcgtggaacggttgcggtgctcgtttatgccaccgcattgtgatacttcttcgagcgtgttggttacaatttattatcctttgtccccaaagttttttgccaacaagctttgatgcttgctttggactcttggaagtcgtcgaagttggtcgaagatttctcgaagtttttattttgatcaatgtattttcaacatgtttcgctttatttcccgttttcaattttttttcttttttcttttatctttcttctttaatgttgatgtattgtattctccaagttagaagtgttttcaacacattccaacttgtggggggtattaaacataaaattaatttaattagttattagaagttatttgagtaagttagttggttactcaaattatagttactagaagttatttgagtaagttagtgttggttactcaaattagttattttctatctttatataaatacatcaactttgtaatactttgatcaatgaatgaatgaatgaatcttaaaactattcatctatcttccatttttttcattcttaatactaacgaacaaaaattatgaagtttacttgtgaagggagtgagaaactagttgttgatagggccaaaattctgcttaattacttccggaggagctacgttgctggtccaatatccactgagaaatgggtacaaagatttactctcatcaaacagatggaaaaagtttataaagaatatttttatagtttacgtttaaaaactgcttcaaaattataattttgtcaattatttgaagttaagttgtaaaatatatcttcctcaattttctaacacagttgttaatgtaatagtctagtgccggcttcgaccatatcataaacaaaattaatttaagtgcacggaaattaacttgtaattattcctagctagccctgttggggtaagaaaatttagtcgagcagcaaggtgtttgatgatgttctccagatccaaaaatttcatatatataattgaaaattaattttgttgcttattataagaaagtgtacagttaattaggagcatggttattaatgttagagatattaatccacgacctattagttactttaattaaaaataacatttatttagcattctcattgtttctctatctctttttcatatttttaataccttaattgatagtatattttttttaaaaagtattttgtttatattatgtagtgcttctttattttattgttataatttgtagtaataggaaaaaaatcaaatagaaagtagggatttggtttttaaaactcaaaataaaaaataaaagttaaaaaattaattcaatctcaatatacagtactcttgataaattttataaaaaagtaaaaaaaacagattaaaaataatttatataatattatatttctttaaaaatttgcttaatgccctaaagttcctaaccacggtcatatatataaatttattttttatgaattatcatattcaaggttatcaaattagaaagtttacgcagactcgtaagagttttatagactcgactcgtagattcaactcgtagactcgtaagagtctacttcatataaaaataataacaaaatatttgtaaaaaacatactaattaaacatttcaaatatataataaagcaaaacagtaaatcataaagttccgaatatttaaaccaagtctagtaataatacattacttgtagaggttatagtagtggtagatcattctcatcgagagtttgatattattagagaacaagagatttgatattattaaatgtgagaattttgtatttgagaataacatgctcaatgaagatatgttgaatgctgaacacacataaaacaataaaaaaatgacttacattttcgtttaatatttttaacttgctaactcgttgactcggtgataaacttgagagtctatcgagtttactcagaatttatagagtttactagaatctactaaaaaaaagtttactcaagagtcaactcggagaggacaagtggactcgtgaactcgtaagaattaacgagttaactctagagtttgataagcatgatcatatcttttaagtaattgtatgcaaaattgatgtgttagatttatgtcacaagatttattctttatatttatatctatattctatatttcaatactaggggatcggaaaaaagaatttaattttatatcgttgctgataaagaaaattgtagacgtactgaatagaagattgatatttagaggtgtcaacgcattctaaatagtcggttatgttaaaatccataatatataagatatctatatatcaaatatcttttatttctaaattgctaccttttctgaaagatgctactaagttttataataaaattagcataaaataaaattttcacctcccaattttaaaccgaactaacactaagttagcacttttaaaatataatttgaaataagttctatcaaaaagttatgttaaatatatttaaaaaaaaaatttactatctacaatagtcttactcaagtaaagtgagttatttttaatattaatagagaatatctgtggattatactaaaaaaatattaattttagtttgattcgcattgatccaattccaaccatggaactgatgactcctgttaacaacgccttgttttttcaaagattaattttattatttatttattatatcacatattatatttaagcttttctctctttattttttattttcttttacaatagtaaacaattgggattgaacataaatttttatacatctatataccccgaactctatttttattaggctgacactaacgagttggatctcaatcacctctattatttatttcttcatattcttttcgtataaactataaagggatatttaggtacaaataaaaaaaaaaccctagaaccttcaagatgttgtagttgaacgcatgtatcacgtgcattaattagtctctatctatccatattttaggtcacgagagtggaccccgaaataaattgcaattattattttaaaaattatgctaaattaagaacatttatttatacattaatgcgtttttattgttaaattctaaaaattagcctacttaatatattttaaaaaataaaattatgtttaatctaatttctgttgtttatactaaaaataatgatttctgttgaagagaaaaaagagacaaaaaaaaaaataacatttaggaatactacaataaattattggaccgttaaaatcagcttacagctcactaaggttttgtcctcaagccaaataattgaggagtaagactatgaatatcaggaatagtttatttaataaaagaatttgtgtttgatttttaatctagtaaagtaagagaaacttgtgatttcttacacaggataacacttttgatttaattttgagataaaagtgatacgtgtagatctaggaagaggcgttgcctatttaaagaagctaaccctccacaaggaattaaggtgcaagagtttgttgtgagctataagctagtttatcgtgaggagaatatgaagttgtttgttttctttgttgctgcagtagttttggtagcatggccatgccatggcgcaggctaccaaaggttccctctccgaatgaaaactggctatggtgagcgttcttcggaggtaaaatgcgcaagttttaggcttgctgtggaagcacacaacatccgagcctttaaaaccattcctgaagagtgcgttgaaccaacaaaggactacattaatggcgaacaatttagatcagactctaaaacagttaaccaacaagctttcttttatgctagtgaacgcgaagtccatcacaacgacatatttatattcggcatagataacaccgtactctctaatatcccatactatgaaaaacatggatatgggtacgtatgctcttaaatctttattttcctcagttttccatttttgcatgctaattactagtttgtttccttatttgtcacttattttcaactattttttgtattgatcgagtttcggtttggccctgtcaacaacatgtgtggcaattaaggttaattaatctgtcatgcatatattattatttgaaatctaacaattcgttgtggatctatatatgcgtgtgttcctttgttaacatttcagattatccaacccttctgacaacatgaagcaatgttaacaccatgaagaataatgcgcaattatatttgaaatctaacaagatatatagatgttaaccatccatctcaatttccaggagcatatagatgtacgtagggaagagaaattaaagacagagaaaatatataaaagaatagaaataagatgaaagaaaaagtaaagatataattatgattaacaattggataaaatggtattatgttccagtcttccagaccagccatattgtcatgattttgttagggaaagccttattgtcatgataagtatgaaaaaaatatatattattatttattaattaaatgttagaaaatataatagtgattttaatataattattagagaaatagaaaattcatcatatgtgacaggttttgattggataaaagtattgtaaactttcttattacaatgtacgggcatggactatttattcatatataagtgtaaagtgctttgaggatggtgttcattgactcaatatacgaataatttattgatagggtggaggaatttaatgaaaccttatatgatgaatgggttaacaagggcgacgcaccggcattgccagagactcttaaaaattacaacaagctgttgtctcttggcttcaagattgtattcttgtcaggaagatatcttgacaaaatggccgtaacagaagcaaacctaaagaaggctggcttccacacatgggagcagttaattctcaagtatgtttccttttttctctctatagatataacttatttatcttttatatataacaagtgctgttggttaacaagtcattgatatcaataatactgaattcaaccatttcaaataatgtgatgagtttgaaatactaattggatctatttttggttggtgtagggatccacatcttatcactccaaatgcactttcatacaaatcagcaatgagagagaatctgttgaggcagggatacagaattgttggaatcattggagaccaatggagcgatctgcttggagaccacagaggcgaaagcaggacctttaagcttcctaatcccatgtactacattgagtagtaccttcacctctctcaacaatctagctagagtttgctcctatctatatgtaataaggtatgctgatatgcactattcaaataggagcattagctatgtttgttaatgtcactttatgttatgtgggtaagtcacctaagacactccacgtacctacttgttgtctcttacgcggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcacaaaaaaacaaacacaaaaagggagtgcattttccagggcatttttgtaataaaaaacagttaaaagggagtgcaatagaaatataggggtgtggaaatagtgatttgagcacgtcttgaagcaaattgggttgttcagttttgggctggccgaagaaaattaaaagatagtggagctcaacccaactaattttcttctattccactttaatctaaccctcaattaattagtttaattttatgactaatttttttaatgcacatacaatgaattctaaaaatttacttcacacacaaactatcaatttatataaattcattatacttatttcaaaagcgaactaattataaactctttgcattattttggtttagctagaaatacatgcaattccacaaatagttacaaacacagttattcgagtgatggatggatgtctcaattatggctgcacaaatacacgagttgtgccttagggagagggggcgggggaatcatgcttttgggtggaaaaaattcatttctaatgctttgtctgtttcactgaaaagataattagtctattcataagaggagatgagatcatatgaaaaatataatcgattctttcatttgtttggactagtagtcatccgtgaaagttttgggtttaatattgatattttaacaataaatccaataaatcttagtgtaatgttagatatattgattttttttttggaaaagaagtgtgtgcgagctgcttatttcaaagaatagagagatgaaaaacaacttcatgtttactacgtgtatacatttggactttggagtatctggagaaaggaaggaataaaagtatgcacgtatcatgtatggttccatatattaactttatatctcgtttaatttagaacgaaaagggaagaaaattagttttttttttctttcaatacgaagataaaaatattataatttatattatatctcatgttttactattaaggcattatctttttcctctttaattgtttttgtcatgaaataacataaaaagtcattttgttgtttttttagttttaatttttttatatcaaactccaactatattttgcttttaaaatataaactgtagagatatgccgtcgattttttaaatataaaataccagcaaggaccacgagcatttatctagtctctatatatttaaaagttaggactagagtggacccaacagtaaaagcgactgcaaatatttttgttggcatgataaatgattgtgaatgagttacgatttttatattatagcagtatttaataaaaagtaaattgaaatttgaaaatcaatactttaatgatataggtatgtttggattaatttctcccgaaatacttctagaagataaataaaaaataactttttgtaagttaatttttgcactaattatagaagctgtatcacataatttttttaaagataacttttaacttatgaaaaattaattttaatcaataaagaaatttatttcatttcttgtttttgaactttagaagtgtttataaagatgtttatataaacatactgatatcaataaaaattagaatgaaagttaccaattcgatatatattaataaaaaattataaattatacatcacgtgtgctgattttaaaaagtttttttaaaaaattattaaaatcgtagataacaattacgacttttgtatctcattcgttaaaataaatcataagactaaaattattttattttgtttcattttttcacactgttttcttaatacttaaaataaaataatagttttaagaagaaaaaaaattaaatttaaataaataagaactaaattatttatgaatggattgaattatcaaatttgattaaattgattttaaatttgatcaaaattgaatcaagctggtctataaacactccaatgtagagggcataaccttccgaattggatgctttatttccggagtataaaaaaagaaagaattaaaatctaaaaacaatgtcaacaaacattgttaatttgatatgattaccagatataatcttatcttttaaatacgttattttttggagatccctataatactcatgtttgtgtaaatgaaacaaaatatacgtttaaaaagatgaaatccttaaatgagtcttacccggttgaaagacactctgagttcaccaatgtaatcaagcaaaaaaaattaccattaacgtaagtttacgaatccttaattcactaataaagacaaacattaagttaatggggataaattaaataactaattgtttttcatatatgttttattcattgttattatctacaaatttaattaaaaagttatatatatattacagttcacagatattataatgtctatataaagaagaaaaccctacgccaggaagatgcaaaagttagtagtagtgaagaaaatgaaagtccttgttttctttgttgctatagttctagcagcatggcattgccatggctcagaccatgaccatgaccatggccacacctaccaaattttccctctgagaatgaaaactggccatggtggccactacattccggaggtatcatgccaaagttggaggcttggtgtggaagcacacaacgtcattgactggaaaaccgttcctcaagattgcgagggatatattgggaactacatgcttggcgaacaatatagatcagactccaaaatagttaaccaacaagcttatttctatgctaaaaccctcaatatcactgccaaaaccgcatgggtgttcgacatagatgagactacactctctaatctcccttactatgctgaccatggatttgggtatgactttcttttgtttgtctatgttattgttagttatttacttacagtaaattacataaactttcttagatttaaagaaattatacctgtacgtctttcttccttttttgacacacctacacacttttcttaatttttaaaacatacacaaactttcttaaatattattcagctagttgagtgaagtcatatgcaacatttattgagtatttttcttcaaaaataacgtggttttcctcctttgagtataaaacatttttattaaaaaaatattttgcaacaaaattcaatcaagttctcacaccagtaaatgagtcaaatgaatgtcgagtaacaaaaccgtagaatcaatctcataccaacaaaagcataagaaaaatgaacaattataagttttccaaatcaaatgaaattactccaaccaaattattatgatccaacacacaaaatcacctaatcaatttcataccaacaataataacattaaatctaaatatattttaaaaatagagaaaaaaacatgtgttatttctttaaatgtgaaaagaattttgtatgagttttaaaaaagaagaggaagcatattaaacctacagggagatttttgtaatttatttttacttatttgtcaaaaaatttcaactgcgtacttttagtaagttgttagttaattgcatatataatcacttaaaaaaaacatatacctttttcttttaacgttagatgaccacttagactaaataataataactcaagtgaatttgaatctttaaagctaggcatcggataaaacaaagtgttagctaaaactaaaagaaataataacagaccttaattttttttcttatgtgcagtgcaggctcgaccgattctgtgattaattagtcagatttattttcttaaacctgactatttctacgtatcccaattttttttgtagtggtttgtgttaaatcatctaaattaaatatctgattattacattgatttttgggatcgaattattaattaaacggttcaattttattttatttttcatccagctaaacattcttccgtatacagtatatcttatgagaaaaatggttattaacattataaaaagttttattatccaattataattaataatatttgtaaatttattgaattttaaaataattattaaattcatatctcattatatatttatagttaaacttttttacttaaaacaataattaatttcccgatcatagggtggagttatataatgaaacatcgtttaataaatgggttgaccttggcgaggcaccggcactgccagagagtctcaaattgtacaagaaactgttgtctcttggcatcaagattgtattcataacaggaagaccactggatcaaaaggctgtaactgctaccaacttaaagcttgctggatatcacacatgggaaaagttaattaccaagtaagtttctttttctctatattttttttataattcc

SEQ ID NO: 29 - protein, 254 aa, Glycine max
MKLFVFFVAAVVLVAWPCHGAGYQRFPLRMKTGYGERSSEVKCASFRLAVEAHNIRAFKTIPEECVEPTKDYINGEQFRSDSKTVNQQAFFYASEREVHHNDIFIFGIDNTVLSNIPYYEKHGYGVEEFNETLYDEWVNKGDAPALPETLKNYNKLLSLGFKIVFLSGRYLDKMAVTEANLKKAGFHTWEQLILKDPHLITPNALSYKSAMRENLLRQGYRIVGIIGDQWSDLLGDHRGESRTFKLPNPMYYIE

SEQ ID NO: 30 - DNA, 10328 bp, Glycine max
  source 1..10328: mol_type = genomic DNA
ttaaaaaataaaattatgtttaatctaatttctgttgtttatactaaaaataatgatttctgttgaagagaaaaaagagacaaaaaaaaaaataacatttaggaatactacaataaattattggaccgttaaaatcagcttacagctcactaaggttttgtcctcaagccaaataattgaggagtaagactatgaatatcaggaatagtttatttaataaaagaatttgtgtttgatttttaatctagtaaagtaagagaaacttgtgatttcttacacaggataacacttttgatttaattttgagataaaagtgatacgtgtagatctaggaagaggcgttgcctatttaaagaagctaaccctccacaaggaattaaggtgcaagagtttgttgtgagctataagctagtttatcgtgaggagaatatgaagttgtttgttttctttgttgctgcagtagttttggtagcatggccatgccatggcgcaggctaccaaaggttccctctccgaatgaaaactggctatggtgagcgttcttcggaggtaaaatgcgcaagttttaggcttgctgtggaagcacacaacatccgagcctttaaaaccattcctgaagagtgcgttgaaccaacaaaggactacattaatggcgaacaatttagatcagactctaaaacagttaaccaacaagctttcttttatgctagtgaacgcgaagtccatcacaacgacatatttatattcggcatagataacaccgtactctctaatatcccatactatgaaaaacatggatatgggtacgtatgctcttaaatctttattttcctcagttttccatttttgcatgctaattactagtttgtttccttatttgtcacttattttcaactattttttgtattgatcgagtttcggtttggccctgtcaacaacatgtgtggcaattaaggttaattaatctgtcatgcatatattattatttgaaatctaacaattcgttgtggatctatatatgcgtgtgttcctttgttaacatttcagattatccaacccttctgacaacatgaagcaatgttaacaccatgaagaataatgcgcaattatatttgaaatctaacaagatatatagatgttaaccatccatctcaatttccaggagcatatagatgtacgtagggaagagaaattaaagacagagaaaatatataaaagaatagaaataagatgaaagaaaaagtaaagatataattatgattaacaattggataaaatggtattatgttccagtcttccagaccagccatattgtcatgattttgttagggaaagccttattgtcatgataagtatgaaaaaaatatatattattatttattaattaaatgttagaaaatataatagtgattttaatataattattagagaaatagaaaattcatcatatgtgacaggttttgattggataaaagtattgtaaactttcttattacaatgtacgggcatggactatttattcatatataagtgtaaagtgctttgaggatggtgttcattgactcaatatacgaataatttattgatagggtggaggaatttaatgaaaccttatatgatgaatgggttaacaagggcgacgcaccggcattgccagagactcttaaaaattacaacaagctgttgtctcttggcttcaagattgtattcttgtcaggaagatatcttgacaaaatggccgtaacagaagcaaacctaaagaaggctggcttccacacatgggagcagttaattctcaagtatgtttccttttttctctctatagatataacttatttatcttttatatataacaagtgctgttggttaacaagtcattgatatcaataatactgaattcaaccatttcaaataatgtgatgagtttgaaatactaattggatctatttttggttggtgtagggatccacatcttatcactccaaatgcactttcatacaaatcagcaatgagagagaatctgttgaggcagggatacagaattgttggaatcattggagaccaatggagcgatctgcttggagaccacagaggcgaaagcaggacctttaagcttcctaatcccatgtactacattgagtagtaccttcacctctctcaacaatctagctagagtttgctcctatctatatgtaataaggtatgctgatatgcactattcaaataggagcattagctatgtttgttaatgtcactttatgttatgtgggtaagtcacctaagacactccacgtacctacttgttgtctcttacgcggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcacaaaaaaacaaacacaaaaagggagtgcattttccagggcatttttgtaataaaaaacagttaaaagggagtgcaatagaaatataggggtgtggaaatagtgatttgagcacgtcttgaagcaaattgggttgttcagttttgggctggccgaagaaaattaaaagatagtggagctcaacccaactaattttcttctattccactttaatctaaccctcaattaattagtttaattttatgactaatttttttaatgcacatacaatgaattctaaaaatttacttcacacacaaactatcaatttatataaattcattatacttatttcaaaagcgaactaattataaactctttgcattattttggtttagctagaaatacatgcaattccacaaatagttacaaacacagttattcgagtgatggatggatgtctcaattatggctgcacaaatacacgagttgtgccttagggagagggggcgggggaatcatgcttttgggtggaaaaaattcatttctaatgctttgtctgtttcactgaaaagataattagtctattcataagaggagatgagatcatatgaaaaatataatcgattctttcatttgtttggactagtagtcatccgtgaaagttttgggtttaatattgatattttaacaataaatccaataaatcttagtgtaatgttagatatattgattttttttttggaaaagaagtgtgtgcgagctgcttatttcaaagaatagagagatgaaaaacaacttcatgtttactacgtgtatacatttggactttggagtatctggagaaaggaaggaataaaagtatgcacgtatcatgtatggttccatatattaactttatatctcgtttaatttagaacgaaaagggaagaaaattagttttttttttctttcaatacgaagataaaaatattataatttatattatatctcatgttttactattaaggcattatctttttcctctttaattgtttttgtcatgaaataacataaaaagtcattttgttgtttttttagttttaatttttttatatcaaactccaactatattttgcttttaaaatataaactgtagagatatgccgtcgattttttaaatataaaataccagcaaggaccacgagcatttatctagtctctatatatttaaaagttaggactagagtggacccaacagtaaaagcgactgcaaatatttttgttggcatgataaatgattgtgaatgagttacgatttttatattatagcagtatttaataaaaagtaaattgaaatttgaaaatcaatactttaatgatataggtatgtttggattaatttctcccgaaatacttctagaagataaataaaaaataactttttgtaagttaatttttgcactaattatagaagctgtatcacataatttttttaaagataacttttaacttatgaaaaattaattttaatcaataaagaaatttatttcatttcttgtttttgaactttagaagtgtttataaagatgtttatataaacatactgatatcaataaaaattagaatgaaagttaccaattcgatatatattaataaaaaattataaattatacatcacgtgtgctgattttaaaaagtttttttaaaaaattattaaaatcgtagataacaattacgacttttgtatctcattcgttaaaataaatcataagactaaaattattttattttgtttcattttttcacactgttttcttaatacttaaaataaaataatagttttaagaagaaaaaaaattaaatttaaataaataagaactaaattatttatgaatggattgaattatcaaatttgattaaattgattttaaatttgatcaaaattgaatcaagctggtctataaacactccaatgtagagggcataaccttccgaattggatgctttatttccggagtataaaaaaagaaagaattaaaatctaaaaacaatgtcaacaaacattgttaatttgatatgattaccagatataatcttatcttttaaatacgttattttttggagatccctataatactcatgtttgtgtaaatgaaacaaaatatacgtttaaaaagatgaaatccttaaatgagtcttacccggttgaaagacactctgagttcaccaatgtaatcaagcaaaaaaaattaccattaacgtaagtttacgaatccttaattcactaataaagacaaacattaagttaatggggataaattaaataactaattgtttttcatatatgttttattcattgttattatctacaaatttaattaaaaagttatatatatattacagttcacagatattataatgtctatataaagaagaaaaccctacgccaggaagatgcaaaagttagtagtagtgaagaaaatgaaagtccttgttttctttgttgctatagttctagcagcatggcattgccatggctcagaccatgaccatgaccatggccacacctaccaaattttccctctgagaatgaaaactggccatggtggccactacattccggaggtatcatgccaaagttggaggcttggtgtggaagcacacaacgtcattgactggaaaaccgttcctcaagattgcgagggatatattgggaactacatgcttggcgaacaatatagatcagactccaaaatagttaaccaacaagcttatttctatgctaaaaccctcaatatcactgccaaaaccgcatgggtgttcgacatagatgagactacactctctaatctcccttactatgctgaccatggatttgggtatgactttcttttgtttgtctatgttattgttagttatttacttacagtaaattacataaactttcttagatttaaagaaattatacctgtacgtctttcttccttttttgacacacctacacacttttcttaatttttaaaacatacacaaactttcttaaatattattcagctagttgagtgaagtcatatgcaacatttattgagtatttttcttcaaaaataacgtggttttcctcctttgagtataaaacatttttattaaaaaaatattttgcaacaaaattcaatcaagttctcacaccagtaaatgagtcaaatgaatgtcgagtaacaaaaccgtagaatcaatctcataccaacaaaagcataagaaaaatgaacaattataagttttccaaatcaaatgaaattactccaaccaaattattatgatccaacacacaaaatcacctaatcaatttcataccaacaataataacattaaatctaaatatattttaaaaatagagaaaaaaacatgtgttatttctttaaatgtgaaaagaattttgtatgagttttaaaaaagaagaggaagcatattaaacctacagggagatttttgtaatttatttttacttatttgtcaaaaaatttcaactgcgtacttttagtaagttgttagttaattgcatatataatcacttaaaaaaaacatatacctttttcttttaacgttagatgaccacttagactaaataataataactcaagtgaatttgaatctttaaagctaggcatcggataaaacaaagtgttagctaaaactaaaagaaataataacagaccttaattttttttcttatgtgcagtgcaggctcgaccgattctgtgattaattagtcagatttattttcttaaacctgactatttctacgtatcccaattttttttgtagtggtttgtgttaaatcatctaaattaaatatctgattattacattgatttttgggatcgaattattaattaaacggttcaattttattttatttttcatccagctaaacattcttccgtatacagtatatcttatgagaaaaatggttattaacattataaaaagttttattatccaattataattaataatatttgtaaatttattgaattttaaaataattattaaattcatatctcattatatatttatagttaaacttttttacttaaaacaataattaatttcccgatcatagggtggagttatataatgaaacatcgtttaataaatgggttgaccttggcgaggcaccggcactgccagagagtctcaaattgtacaagaaactgttgtctcttggcatcaagattgtattcataacaggaagaccactggatcaaaaggctgtaactgctaccaacttaaagcttgctggatatcacacatgggaaaagttaattaccaagtaagtttctttttctctatattttttttataattccaattgttttgttgagttgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacagtcataagacccgtatacaccccttgggtagcttggtgattcttggttgatgcgatatgaattattaatttaaagatataaaagctaatcactatatattttaaatctataataagatagtcgaattattggttatctgttcacaattatatatgcaagtatatttaaaattataaaagagattaacaggaaagtgttataaaaaacataggatgagctttgttaagtagtgataatgttccagcccctataaaaaagtaatggtaaagttgtcaaaacaataggacaaacttaacctaggcccgtgggaaagggacttggaataaattgccaaaagggtatgcctctttctttgaaagaaaaggccagttgtcgcggatggctttgccgaaagcaatgaatcgtgaaaaaacaaaaaaaacaaatatgtgagttaagatttttcgtgtgatatatgtcgttgatattgaagtgcactataatttgcatgttcaactaataaaattttaagagtttaatgggtaactgtatacagcatttgtacacaactaatcaaacatgaccggacggccattagcatattttaagataaaagtcaataaatttctttatataagtattgtgatggaatgatagcttaaaattattttatattattaatatataatttattttttctaaacttttaacaattagtttttgataactcgccgctagaattggtttagtggtaagaagtttcgatattttgtgacaactctttagttcaaactttgttttatatatagctattattatacactaaacagaatataattggttttggttgtaggaatacatctgaataccatggtaagacagcagttacatacaaatctactgagagaaagaagctggaggaaaaaggatacaaaatcattggaaacattggagaccaatggagcgatctattaggaaccaacacaggcgataggacctttaagttgcctgatcccatgtactacattagttgaggtctctagggctcttactagcatgttcctatgaataaaataagatgcatgttgtaataatggagagatcgacttaaaccaaggagcactcgttaattaattgctatgtatgtgactttgtgtgagacaaaaggtcacgtggtaagagactgtacaagcacaacggtgttttatctttcgtggttttaataaatcatgtgtgctataattccgttaatttaattaaggggtgttactttaatttctttttgaagtaaaaatcatgtcacagaatatttaagacaatcggatgagaaaaaacttgacattctcataagataaagaatcttaaaaaagttatatcattttttaaaaaattatccaaatgcacgttagtcatgctcagttatatacgtccctatttttttagtgttattttttttatatagaaactattaaaaataatgaatagaattctataaatgacttatacacaaattgatgtcttgaattttctgaagacacgtgctagctagctagtagctccatccatggattcggtggaaccacccacatataattgcatggataacattataaaactggcaacgagtgtgatttggcagagggcatgtttctccttatgatgtgttttggaacagtagacagaaatgaggaaggaagaaaaaaagtttgacgtttgatctttttcggtcctccatcttttttcataaaatatcttttaattctttatctattaaaattaattatttttgttatcatgataaaataattaatctaaacgtagtggtgatgtgtaactgtcataatttttttattaaactttaaaaagtatagaccccacaactggaaataaaagaagaatgaaatgcatcagcatcattctttaagaaaaatatcccaaaacaaatgaaatgttttcttactacaattcatcaccatagaactttgtaatgtagttatcaacattgtccacggaaaagatcgaatataatgtgatgaaatgaagagaaacaaattgtgcattgttttcaagaccgagtgattggcaccaccaactaaaatgaaagctgaagggaagagagccggtggactgagtattgtgtagtcaactttagataatcgatgaatatgatataattttggaaataaaattccggcttgaagccattttattaaactcaaaattatatcacgtgttcatttattaaactcaaaagaataaaggtgtatgtatagcctactgttcacagatattttatcctctagcggtcgagcaatatttattattttatttttataattataaaatttttatatttcacttcatacatttaaaaatcatttcttcttatttctatacaagtattttcggatttcttcttatccttgttgttacaaaatgataaggactaaaaaacaagtttgaataaaagggatggtgatttttaagtagagactaaaggtaaaatttgttcaattatagacattaaaagaataatttttcctttatttaaagaaaacactataccaagaatatgcaacaattagttgtgatagactgataggtagaagcagtgaggagaatgaaggtcctcgttagttatctttgttgctatcattctggtagcatggcaatgctatggctcaaaccatgaccatgaccatggggcaacctaccaaatataccctctgagaataaaaactggccatggtgggcactacattccagaggtgtcatgccaaagttggaggcttggtgaggaagcacataacatcattgagtggttctccaatattcctgtgcataggatatttagggaactacatgctgtgcataggatttctatatattccactgcatatagacaattgtcaccttcttaatcatgttttcaacaaataaatattatattggtattttaaacttatatataagtttgttgttacatttagtatctgagtcttatttatatgatttaaagttcaagtactttttgtgttttggttttcgacaatttatatcgttgtcagtaaattcatatttgattattgatataatgaatattaatcggggcgcttcttcaacaatttcccattttggctggttgaatttctatttcggaaattgtcgatcaatttttttttattagcgttgtaattcttgtccattgatcggtacgcttcttcaactataaattgtcaataacaaaaaaaaaattgaatatttgatgctgttcaaatttgattgttgtccgtattttttttttctcaaatgcaa

SEQ ID NO: 31 - protein, 271 aa, Glycine max
MQKLVVVKKMKVLVFFVAIVLAAWHCHGSDHDHDHGHTYQIFPLRMKTGHGGHYIPEVSCQSWRLGVEAHNVIDWKTVPQDCEGYIGNYMLGEQYRSDSKIVNQQAYFYAKTLNITAKTAWVFDIDETTLSNLPYYADHGFGVELYNETSFNKWVDLGEAPALPESLKLYKKLLSLGIKIVFITGRPLDQKAVTATNLKLAGYHTWEKLITKNTSEYHGKTAVTYKSTERKKLEEKGYKIIGNIGDQWSDLLGTNTGDRTFKLPDPMYYIS

SEQ ID NO: 32 - DNA, 12018 bp, Glycine max
  source 1..12018: mol_type = genomic DNA
atgaatatgtcactttagtttatgtcataggagttggactgctagtgtgtgcttttgactattatatgtattattttgttgttatttgcttttgtcatttacgtaatgtagtaggggcttttgactattattggcttctttcatttatgtaatgaattaaatggattattataatatgtataaatgcactattgttatattatttctttgttatcattattaagtcatgtatttggacaggtgttaacagttaaatgaataattggattaaacgatttgagcttgaaagcaatgaagacaatgacattttggaatatgcttcttatgtggctgcattggttggtgactatgtaataatgcatatatgtaaagagccacgtagaactagtgaacatacaggtcatacatgggtttatgaaatattacaaggacatccaattcattgttatgagatgtttcgcatggaaaaatttatttttcataaactttgcactgaattagttgagcaaggcttaaaggaaactaagggcatgagggttcaagaaatggttgcaattttcttaaatatggttggccatgggttaggtaatagaatgcttcaagaacgatttcaacacttaggagagactataagtaggcatattcatgaagtcctacttgcttgccttaaattgtccttcaaatatattagaccacaagatcccatgtttcgtgattgtgaggtcaaaattaaaagtgatcaacgatattgaccatttttttaaaaatggcataggagcaattgatgatatagatatttcatttatagttagccctgttgaacaagcaaggtttattggaagaaaaagtattccaacacaaaatataaaggttgtatgtgattggaatatgtacttcacttttgtattagctggatgggaaggtactgctcacgatgcttgtgttttttaccatgctctcacaaatgctaacttgaattttccacatcctcctccaggttttttttatatagatatacatgatttataatttgaataaaaatatcatgtttattcacttttttaattgttttattaggtaaatattatttagtggatgctggctacccaacaccaatgcggtatcttggtccttacagatgtgaacgttatcatctccttgattttagacgtaaatttggatttgcaaatgataatgaggtcttcaatttttatcactcaagtctaaggtgcacagtagaaagaacttttggtgtgtggaagaataggtttgcaattttacgtcttatgcctaaatacaaatttgaaacacaagttcaaatagtttgtgcaacaatgactgcataattttatcataaggaatgttgaaatggattttgattttatacgctatgaagaagaagacatacttcttgatgtagattataataatcacaataagactagtttagttctaaatataatttctgcaccagaaatagatcatgttcgagactatattcgagatgaaattatacagtacaagaaaaaaaactaatattatatttattattgaatttgattgactttatatatgttccttatttgtactttttaatttagatacatttgtgtacttatgttaattttaattttttacaatgaatacgagtttatatatctatttaattttaatttatttatatgtatatcaaaaaatagtaaattataataacaaataagtattcaaagacttaaggtctattttaataattttactttcaaaatcaattatctaaaatcacttcaatttaaaatcaattttatccataggagctgcattcaagttcacggtacattgtaaaacgtaaacagtatcatgcgtttgacaagcttgcgttaaattactaccacgtttttctcccatgttaccaaaaaaataaaataaaaaattgctactaccttttacaaatcaaatagtatgaagtaatttgccccacgcagttgtacacgactcatccaaataaaatatttggaacgtctattagactattactctttaacagtctactgtttattttaacggtttcattaataagtcagattaaaaaataatttgtcaaattataattttggatgacgcttacgtgacaaagtggacgtttgatattgaaaaaattaaaattacttatttgaaaaaatataagaactaaacgtcttcatttagaaataaaaaaatcaaaatctcagatttaaaattaatgtggaaccaaaattaataaataaaaatatataaatagtattaaatattttatttttcggtaaaaacttttctgttaattctgtattcattattgtccgtttgtccagctattatactaaaataaaatctgtattcaaattcaatccactcaattcttctttatgaaattttcaatttaattacatttttaaaaattaaactttgagacaactaacgaatgtgtccttttttttgtgtgtgaagactagcgaatgtgtcgattcaaacttaaccactctattcttcgttgtctaattttcaatgtaattgcattttgaaaacatataatttgagaaaactaactaacgaaggtagtacaatttcttgtgaacaatttttaactataatttaattagaatgaattcacattatgagcttttattacataatatataggattaattatgtaggataatatttattaattaatttataaattatatttatgattttttaattatttgacatgacaatgaaaaaaaaaattatagtgataatgtatcaaaattaagttctttaaattatgatactctgctattgaataacaaacacgtaaaacttttgttgtaaatatattctaattaaatcctaacaaaattattaaagtataaatgtaattaaaatatattcattatcattcagttacatatagtcatatatatttaatataattttttataggtacatgaaaattaaagtctaaaattgttggtactcacaagtactttctaagcctattaaaccatgagtgccacaactgataattaaaataaaaacatgagtgccacatgtcatgtcctttgtcagttgtatcatactcaacacagtttaacttttggcataaaaatatatatttgatttatatattaaaaaataaaaatatatgtttcacaaaatatatttcaattaatttttaattttttattacttaattttttatgatttaataaataaactttttaaataatttttattatttttttgaaacactatttgaaataatattttttaaatattaatttttaaatttttaaaattttatctttttatttttgacatatttatcaaattttttatattttctaaataaattatgatttattattttttattttaaatttttacttacttcaatcaagttttacaaacacttatgatataataaattaatttttcaatttttaattaccaattaagtttttaactttccattaactcattaatttttttagctaattttattcaatataatacattaaatagaaagcagcctcgttggttgagaagaaagaaaattttctatgccacaaaattttaatcatccattcaaactcaaattttcaaggtaattgcattttaacaaaaaatattatgttttcataatgaaagaaaattttacattattaattaatcataaataatataatcatacattataattttattaatttttattaatttttattataattatcttaattggtttaccgtgtaaaattattttttacaccaattataaatattaaactctttaaaaattaaaatgaagcaactatgagtgtagtacaatttcttgtaaaatattaaaagctaattaggacactcataatataaaaaatttaatgtttatatattattagtataaaacagttatatcttaaatcaattaaaagttattattattaatataaattttaagataaatgtgatctaaaaatttactatatatatatatatatatatatatatattattttagtataaattatatttaaaaatttttaaaatttttaaataatacagtgtatcaaaattagacccattaaattaaatatttgacacgctataattaaatgatgacaaagcatgaaaagcttttacaccaaatatattctaattaaatattaaaaaactgttgaaatatatttgagaaaaacctgattggctgagaagaaagaagcgcgtcaccgtcatcctaccttttttcagtatcttccttagtccaccgtccaaccaaaccaaccccacgttttcgtactccccaattcaattccacgctgaagacggaatattccaaaactccgtcacttctcaaccccgaacaaaacaatacaaaacccaacatgtgattagattattttgaatgaaattaatttaataaaacttattttgattataattaaatttgaagtgatataatttatatttaaatatttcattattatagtaaaaataacttagtaataagatttagtataatgttttcttaatgcaataactatttttgttttaaatttgaatccattttggattaattttatttttgtcaatttgaaattaagttcatattagttttcgtgtaggattcttctaaatcacgttaaaatatggttaattaattttagataattttattcacatatttaattctactttaaagaattgaatttagatctaaaatccattgaatcaattttaaatgttttagatcatataatttttactaaattttactaccaacatatttttaaaacacaaattctatcactttcaatcaatattttatcaaaattaatttcattaacattcatattaatatgattatgaaaaatccaagtgaaacaaaacatacccgcgttctcctctatcacacgttacgtcgtcgttaagtcccgccactctctcctctccataggttactgttccacacagaaacgcatatacacatttcatttcctatttcctttttgttcatttctttctttttcctttcctccacaatttgcatttcccctctcttcgtttccttttctcctcattttacatttcccctctttcgattctcctaagaggggaacccaacccaacaattcctccatcccttgcattggtcacaccatggttccctctgcctttctgttccatttcctcttcttcttttcactcctcatctcgttttccttctccgattccgattccgattccgattccaattccgtaggccccactttccaccgcgtgttcccgcggccgctgatcgtggagtacccggagttcgaggcggggctgcggtgcggcgcgtggcgcgtggcgggggaggcgaacaacctgggcgcgtggaggacgattccggaggaatgcacggagtacgtgaaggagtacatgaccgggaaggggtacgcggtcgacctcgaaatggtctccaaagaggcggaggagttcgcgaggagtgtgcctctcggcagcgacgggaaggacgcgtggattttcgacatcgacgagactctcctctccaatctcccctattatgccgcacacggatacgggtattcccacaaaaaaaaaaatattgtattttgttttgttactcttttttggatcatgatgacattgttggggactattcatctgacattggtgcgtaatattcaatgctgataactaatcaccgagaaaaaaatccactgacaacctttagggtccgttcggtagagtggaaagaaatgaaataaaattgaattgagatgaaaattatgaattaaagtagagttgttagagtaaatcatgttaattagctcgagtattattctctttcttaattagtttttatgtttatacagttttactaacgttgagggactattttaaagaatttttcaatactggggttatttcgtatatttttaatgcttgaaggattacctgtaggttaagcatataagttaatttgaatttgaataaggtggaaaaaatagcagagataaaatatgaacaatgatgcattttctattgttcagcccattttttttttcatttttttaatgtaaatttcaaaattttctcttttatttcgttttttccccctcaaccaaacaaaggtttaagatagagacattaatttagagatataaaatcctatcatttaattataatggtgttgtaaagatagtttgttggctgaaatttagtttgctttcatctgaaagctgaaaaatagggtaattcggaaaggacataagaagtgttataaattattgtttaacaatcaagagagattgaagatactgtaatcctatttggagttgggaattttatgcttttgtgggatttaagaactctgttttgtactctttggttaaaaaaaaaaaaagagttaaataaccccaaattcatgcttaattgcccctagttttgtttggataatgttggagcttgaatttgagtttacttttacaccgaaatagcttttaggccaaaagttctgtagtgtgaccatttggaggggttaatcaggttttgatgttttcttattctttcctgtgaatattcctctaggaatgaatgactctagacagtattcttctgtatctatcatgctaacttgacttatttttctttcttcctaacatgaacctttgtttgcatttgcttgtaggcttgaggtctttgaccacgagaagtttaacaattgggtggagaagggtgtagctccagccattgaacccagtttaaagctctatgaagatgttttgaatctgggattcaaggttattctactgacaggacggagtgaaaggcacaggagtgtcactgttgataatttgatcaatgctgggtttaaggaatgggaccagctcatactaaggtaattcccaaaacatatttgctaattttagaatagcaaatgaagtttgtatattaaataaatgttgtaagattggaatatatgtgtatgttctgcaaccagttctgttttacatgaacaatttaatgcttttgtaatctagataaattttgtcattttgtgtttaaaccaccatatcattcagatgcattgtgcattgtaccagtctctcgaagatttggcatgttttcattaagaataacctgaaatgataacattattgtaccatttcctttatggttcaactgttttgtctcctagcatgcttaatgctatcataaaaaattaatataccatggataaattaaagacaatgtttcatgacccagaacagaacattgaagaaggataaactctgccttggtttattttaggaggaatcttgcaaacaacccattgattccagcctccttttttcgatggacagcagaatctgcttacctgaaatcatcttggcgctaatgtttcagaacattagaaaagttacgctaagagttgatattttctttttgtagggatagcttacctttagttcaagttaagttagtttagttgtttgcaagtgagaagatcaagtataggaaaaataaagcaggatggtacaattaatagaactacaaacaaataaatagcaaatatgagaataaaggcatggatttcagatattttaatgcattgcatggctctagacaaggtcagatggagcatttcatatagatttcaagcagcctagttaatggtgattagtgtagagaatatagttttacaaaatgatttttgctcattagcaacctcagctaatgtacaacttctaccatagaggaatttaatgaatcctgattgcacatgctaaggtcacagaggatttccttatatccataggagtataccaacaccaagggttaattttaagcccttatctttctacctagatttgtgagctatatctaggagctattacctcactgcacacttattttcaaatcatatatagtcccttgttagagatgcgtttgacagaactcaataacgttgtctagtccatgaagctgatccgaacacctatgccactgggaaaaggctttttgtgttgttattttgtatgctacagtaattggaaagatttcatgctccttccttgttatggttctccctagtttcaatcacttagtgtttctacagagttaacagtatgcattgcttttagtttctttagttcatatgtgtactccatataaaatccaagtgaataccatgtcagcatcaatattggcatattttgtgaatgactagatgctgttgttggatgatatttcagaaagctattgaatggttattatattttaggtagatgatgatatatttgtcttaatatctttggagagctaatgttaaaatttattaattaatttaatgatacattctcgattgttttaagtaaaacaaatttctctccttggcatattatctcattgttgtctgcgaatttgtgttttcgtacactgatagaactaatccatatcatatttttgggccgggatgctgacaatacatacatatattatctatgggaaagcattataagttatgaaactgattcatatatacttcttatcaagggaatgagtacaaatgtggccagtcttgtcttgtattttccccttattttgagatgtttctcttcagcgattcatgatatagggtcatattttccttttcatttgaatcatatgcctttaatatatgttctttgttgtgtctgtaaattctgtatggtttgccttcatgtagattttttggattaggcttcactgttgctgttaataaattaacttgctattatgaaatgggaaaaaacaggaaaaaaataagaataaatttaacgcattccactacagcaccctctacaatatttacattccttatttcctttcataattgattctttctaagcaattcatttccagaatttcagagttccacaacctcttctgtttgttctgtgctggcatagacctttaatcagatcttttatggatcgatgaatgttctgaaattttgttcaagtggactgaaatactcaatttttatggatgaattgtgaagataccagaaatttagccctttatagtacaattttgcatctcaatttagagtaggttagatgagtagattttaattaattttgtcatgtttcctaatcataggattggattaattggcaggggagattagatgtcccaaactctatatcatgattctatataatgtagacagtttataggggtacacaaggcaatgatagttgtctacttattaccagaaataactacatcatagtccacttgtattctggagtcttcttcatatcttttggcactagttaattacagtatgcctggttgtagccccattttttgtggttgatattaagtcattaattttgacttatttagttaccaagttaatttaccctcctgcctttactgttcctttctctgtatcctttatgctgtatatgcaaggtgaaagcttctactttgagaagttttctttgtacatgtaatcatgtacaattttaggatcatcatgcttgtattttgcactctgaagctgtgtcacttttattttatgctaatttttctgaatctgttattttgtaatgttcagaaactcagatgatcaagggaaacgagctgtactctacaaatcagagaagaggagtgagatggagaaagatggatacaggattcttggaaattctggtgaccagtggagcgatttattaggttcttcagtatctgttcggtccttcaagcttccaaatcccatgtattacattccatagtaaatgtctcattcatttgcaaataattgctgtttgaattgtacataacgaaagtggatcaattcgtatactgaatatcattgcaaataattcataaactgaatgtcattgaaattctaatacataccgactaaattaaacttgtgcgaaaactcatgtaacatttgtcagttgttagttaacatgattgtgcatggttttcttctgaatcataactatcacttggtaatatgacttgggatagcttttaaattttaattatacactcagcttccttgacaactaaggaggcatttgcggtttacagcttttaaaaagaaggccatacgattatttaatatatcatatgcgtatctttcacctagtaatcaaagacattagtcttgcctcttctaacgtagttttgcggcttgcatacacgatgcgaggaattgaacctgagttaacatgtttatatatcaattaagctggattttatcagtaacaagtcaaaccattttatattgatttttcttcttttatgtcctgtcaatttcgatttaaaatatgattctgctccaatctattctaacctaaattgctttggaatatatatatatatatataatcatgtctagaccattatgatcttttttccgtatttctttttgctcaaactcctcctcatcatcttcttcaagaaattccaatttattaaatgatcaagatatctagacccaagaggtcgccccaaaacacaatctcttcgatgaggaagtccgattcgaagatatcaatcaaaacatggatgactggaacattccagaaatccctcaggatcaactatacgttcctgaaacaataaaagacaaacacaactttgattacataatcaaaacagtcgaaaacaatattcctctaggacaagatataggggaagaatttcatttactttcaaaaaattcaatttatgaacatagccgtaagtataaatatctacacatcggttgtgtacaagtagccatcaagcccttaattgatatgggcatagatgcggcagttttaatgtgtctaagagacattaggcataatcaatttgaagactccctaattggaacagtcgaaaccagcctaggacaaggtccaatatattttaactgttatccaaacaaaacagtgagtctgatggacagaaacattcttgactgtctgttcttaaacatccactttcatggccttgacatgaaagaagggtcaatcccagcagccttaatttataggatccagtacaaggtcatgaatacttgtgcatctagagtcctgttaaaacctcgaaaaggagaaacaaccttgtttatcactgatatgacaaaggctaacgtttctctcccgagaaaaataaaatgggatgaggtaactcttcccgaaagatgggttatggataaggcaacaccgtctattccccgacccgctccaaccatagagcatatcaagcaagataactccggaaaggtagagataaccttcaataggagaaattctttttcatcaaggataaaagcctctaggtctgaatatgagtcagccagaaggtctttttcgactagaacccgttcaattctagtaggactaactagatctgagtcacataaccagttccctactgtaaacctccaaggaatagacactacttctagcatacctagaacaacatacaaccaagaacaagaggacgaccaaaagtcgatccaatccccaacatactcttctatggaaccttatgatgttagctgacaactttagcattgataaggaaacccttaggaaagatttttattctccggagaatgagcctcaaaggagatggtttttccaactttacaagggtacaaacaaaaaacaaattcaagacaagttctacgaatttgtcgaaagaatcaagattaatgtccttttctttgattggtttcatgcttatgccatcagaaatgatatagattacccttggaaacaagacatcataagtgatccaacaacaaatgtcataacaaattggcaagttaaagatggtgagttaatccaatcggaattacctcccgcaacacaatgtcaactaccaaatatcaaagacagtaacaataaacctgtcatggcaattccattcaagacaaaagatgtcaacgaggaagtaacctctaaagacatcaagagcctaatggaacaggca

SEQ ID NO: 33 - protein, 271 aa, Glycine max
MVPSAFLFHFLFFFSLLISFSFSDSDSDSDSNSVGPTFHRVFPRPLIVEYPEFEAGLRCGAWRVAGEANNLGAWRTIPEECTEYVKEYMTGKGYAVDLEMVSKEAEEFARSVPLGSDGKDAWIFDIDETLLSNLPYYAAHGYGLEVFDHEKFNNWVEKGVAPAIEPSLKLYEDVLNLGFKVILLTGRSERHRSVTVDNLINAGFKEWDQLILRNSDDQGKRAVLYKSEKRSEMEKDGYRILGNSGDQWSDLLGSSVSVRSFKLPNPMYYIP

SEQ ID NO: 34 - DNA, 8631 bp, Glycine max
  source 1..8631: mol_type = genomic DNA
gtatcatgattctcggagcaattattgtctaaattactgtgtttcatcgttgattggacaataaatagaaaaaaaattaaaattaactgtggagtcctttcttctcaaatatgaaaacaggtagggaagacatgttttttaatgtaaaaaataaaaatgttgatatattatagaaagtgatgctagtgtaaaaatttttgtatcttttatattatcttcaatgagaagggtataaatcaatgtcagtggtggtcagaaaattttgacatggcaacagtgtaacgagacagaggatagtgtccttgggaagtagtatgatttatgtgcataatgacacgagcaaatgagatgacaaagttacgttgatgtgaccgggcatgaaacctatcctaaaaaatttcaaaataatgaccccgttttcctcgtgctttcaatgcaaatttacttatgttgaaaacacgttaataaaactcattttaaatatttttttatcaatcaagttctaattttttttagtaaaaaaatcaaactcataattttcattagcctttcttaatttttttattaattcaatcttataattttatttattaaatatgtctctttaaattattttaaatatgtctagatttctttagttttccttaattttaacatattcacgcaaaagaagaccataataaagaaaaaaattagttaattttttaattctctaattatgaggttttgaattttttttttaataacattaaattctttaaagtaaagaggcatcacttttgcttcacctaacagacgcatatgcaaataggcatcacttttgctgcactctgaagagggttcctgatatcgctaggaggactcagctttgtcataattcacaagccttagtcagacactcaaacttcacacttgtacgatttgtacagtgtttataaaagttgttaaatagttgctgatagtaaaaaaaggagttttcaccaaaaacaaagaagtttgaaagaggagtacgaataactgataacgattaatagcctttaatttcttggtcagagtaatattttgttgaatgaaatagtaaaaacatatttgtatatcaacaaaattcatatttgtttagttgaagtgaaagttagtgaaaaaaatatttaaattaatgagttggtagataggaattgaagaaataattgtatatcaacaaaattcatattttatcacttattcaatacatacatgtttgtctaatttaattttttacaaacactttaaaaaaaaataaggcttgtaatcagttatgtctaacatgacataattaattatatgtcactttaaatgcatgtttgctttcaaattctttaacgcgattattcatgtcaaaacaaaagccacaattacttgcttatgcttaatgggagtgttggacatggagagagaatctcagatccaagttggataatcctttctaatcttttgaaaagtagtcacaagttgcataagctgggtcacgagggggctcttttggatttttttttttacagtgcatttcacttctttttaatttttatctctaattggcattggaaagctttatgatgaccacttattaagttatagtgcgtgtttgctttaaccaccatattcaagagaaattcattaaacgtacagtgatgaaacctattaaaatttatgatttaaaacaaattataaatgatagtaaaatatgtgttcaaaaatcaaaagaatgtcttagagaacattaaaaaaagttatacctgatttatcttaaagaagaaaaaaaaagttaggccaaaaaaataataaaaaatacagtaatagaaaaatacacgtacaaatcacaaattttaattctctagagtagttgaccagttttatctttcagagaaatgagaaattatataagagaatttctataaattaaataataattagttaatgaaaattaactctggtggggactacaattacaatattcctgaaaattatatatattagaacaaatatcgtataaattataaaagctaaaccaaatatcatattagaacaatacaattgttgaaatttattttaatgacttttataacaaaattaattaattgagtttttgtttttaaaagtataattaatgtagattttttaatataaattcaagaaatttatatttggctttccttacttctgcacctgtgtccgcccagcttatggcaaagggtataataatgtctgtaggtacagaaaagtggaatgagaaaattgaaatttttatttatcttttatggcaaaatgttcaaaaatgtcagagggcagagaaaagtggtatgagaaaattgaaaatcatgaccagcaaacgatggcaataatgttatgagatggaggttggtctcctccgaaattaatggtccgtttagttggaaggaaaaaaaaataaaaaataaataaataaattgaaatttaatttgaaaaaaaatggaagaaacaaaatttttaaaatgttctttattgaagttaactttttttcttccttctatttcgattttttttaattaaacaaaaaaatattttataaacatggtatattcttttttatttcatatattttttcacccaaatatatggtaagtgcaaaatgacacaggccaaatgagttggcattgcatgtcttgattcagttcaagaacgtgcgggtttcaatttttttgcaacgaaagcaaaagttaaatatagcatgtttatatagcaatcaaagttagttttagacctaaaatcatggtgaaaattttaaaaacaagtgattatttgttgctttggcttttaatgtgattttagaattaattatgaaaaaattgaatctaatacaaatatgttattattcgagtttcaatttggtggccgttgatattggagagatgcgcatccaaacatattaataaattttatattggataaaagaatttatactaaacttcattagtaacccactttttaataagcttaattatacattttcctacttaactattattattttgtaaattttacaatttaagatttttttatatattttacttacctgttaaatgtgtaccaacatgatgacgcataaaaaaagtcaagaaaaaaatgaacatcattaatttttttatattttttaacacatcatcacccaattacacacatttaacagaataataaaatacatgaaaattaaaaaaatatgataattgttaagaaaaatggtcacaattaaataataaaatgtataattaaatctgcacaacaaaatatttaaatataaatcatatgattttaaaattaattttacaaaattgatgtatttaaaattaattttagtttgtacaaatacatataacaatttttaaatttttcgattactattttctttaaaccaaaaacctctaattatttacttatttttgttcactctgtttatcttttttttttttatctatcttattactctttgttttttttcttttgcaatcaaacacacccatattccaaagaggcctcatgataatcgctacagaggactcagctttgtcagaattcacatttgtactaagaacagtaaagtgattatataataattaacataggggtatgaatattcgaataactaatactaatgttaatagcctttaattttatggtcaaagtaaaggcctgatactagtctactccattggataccaaagttgcttcttacacctctggattggaatttggaagccaatagtttgttactttgtggtccaaaagttaaccgtacattctgccattactagtaaacgtccaaaatttactaatgattacacctgcaagttgtacggatttcacatttgaagagaaaccacgtactatttgattataaaaatatcattaaaactaatatagtatattatgtattttacataataaatatttattcgaaattaataaatatacaattttaaaattgatattaaagttttcatataacaatttgtgatataataacagtttttaaatattttatattattagattatatcatatacaaattatattttaaatttgagtaataatgttaactgaaaaaacttttaaaaataagttaaaacctttttttttccagtctctcttattctaacgtagaatatgtgtggcaaagaaataaagttttagttttataaaatctcttgcattaaatgaataaattatattttcggtctatctaatatttttcaaatctgattttagtcatcccctaatatagtacttttggattttcatgcggtaaacgtgcctccatacaactagcatggctctgagtcaagtttttgacttctagtatgcaaaattggagcttcagtagttttgtgtgatgcagatgatgccatttgtgcgtgactaatctgcttgttacaaagttccaaaaataatatttttattattattttattcacaaaaactcgaattaagaacttcaaaaaattaaattatgtagcactggaatataaagatctctgtatcatcaattaactataaatcagtttaattataactttaaaagtaattaatataaaataaactatttcattcgaatgataatttaaaattaaatttagatgataatctaaaaaaggaaaaaaggactcatctaaaataattaagagcaaaataagtgatgttgatgaaaaacaattaatagttaatataccagcacattaatgatctgttatgtatatacattcagttttcagcctttgattttttcatcaacagttgagattaattattattttatcttctgaaacaagtgactcactggttgaaagaagctgaatctggcaaactttaaccatacaatcatttaatttaaaaatatttattacattataaaaatgatcaaacttactctatataataatttatattaaaagagagaataaaattaaatatttttgaaaaggataagatacaggaatagtgactaaaaaaaaaaaagcagagtagttcaattattttgttatcctctagagtgtagatgttggggtattggctatttaaagagcaaaagcctgcacttggaagatgcacaagtttgttgagatagctagaagcaataagatgaaaatgaaagtgcttgttttgtttgttgctacagttttggtagcatatgaatgccatggctcagactaccagaagttccctctccaaatgaaaactgggtttggtgggcaatacagcaatgaggtggcatgtgccagttggaggcttggcgtggaagcaaacaacgtagtcaagtggcaaactgttcctgcagcttgcggagagtacatagctgactacgttctaggagaccaatatagatcagattccaaaacagttaaccaacaagcttatttctatgctaaatccctcaaactcaccaacaaagatgtctttgtcttggacgttgacgacactactctctctaatctccagtattttgctaaccatggatttgggtatgtttttttatatctcctctttttggtcaaggaagccctcaattcctatctcccacctaacatttctaaccattaaagtaactataaaaacaaaattattaatcagagcaaagatgggcttgtgtttcttaagcaaaaacaaaacgtcattgacaaaatgtacatcacgatgtgaatagtcattagtaaaagagtaaatgtgaatactttactccccaaaaatataataaaaaaaactgctttcgcgatgttgttcatattattctgaatgtaatttttgactgaggagtaatttggtgatagtgtggaaccacacaatacgacggcgtttaagaactgggttcttgatggcgaggcattcgcactgccagagactcttaaaatgtacaacaaactgttggctctcggcatcaagattgtattcttatcagaaagaccactgagtctaggggatgtaacagccaagaacttaaaggaggttggattcaacacatgggagaagttaattctcaggtgactttccttcttcttttaaaacctcttaatttacattgtaaatgtaaaacattttagaatttggtttagatgtgacattaaaataattgttaatcatgtgatgaacaatatgacactagttgaggttagtacaacaccaagtaactctgaatgaacactttgtcatagaagtttcattttcattaaaaaaaatcaaggtttgagtatggtccttaattttaaaaggctaaaaaaaagtgctgctttaatttggttgttgttgcagggatccatctgagtactctggtaagttatcatttgaatacaaatcagctgagagagagaagctggagaaggagggatacagaatcattggaaacgttggagaccaatggagtgacctcctgggaagcaacaaagggactaggacttttaagctgcctaatcccttgtactatgattaagctctcaaatcaagcctctaagttgctccttaatttctatgaaataagatcctgagtattattacttagtcatgtaggggcactgaacaatgttgttttcccttttcgaggctttcatgaattatgagctcttgttccattaagaaattgtccgttccaagttaaaccagtttagtattattattaagtaagtcttaaaaaatggaaggatataagtttttaaaacgaaattgtctactaatgtcttggaaggatattcataaacctatcccatccaatttgaacattaaagataaaagaaaacattaatttctttgtttaattagatactattttggatttaagtttgaaaaatgatccaatataatcctaattggaaatcgaacaaagtaaagctagaaaaaataatagttttttaaattattattattattcaattagatagggttttggatttagatttaaaaattgattcaatacaatctaaatcaaattcgttcacatacataattttcctttatcaaattattatactttcataatttcgttttttttcaattatgtctataacatggatgaattctatttcaacatgttttttttatttgaaattgttttcagcaatatctattatttattatctataaatgataaatgaaattttaaattaatatttattttgatgactgtatacttttagatttatttaataaaaattacacgtttaaaaataaaacaaaaatcaaaatatctaatttaatctaaaatacttttaattagattatattaaatttaatattagtttgatttaaatctgaagtgaatcgcgtaattttttttattaccaaatcaaacaatttaaaacgaatagcgaatagtgttgaaaatattcattagttaataatagattaaaggaaatcgtaaaaataaatgcaatattatctgaggttggcaaatgggccaatagatgattaataggccattcgtacttgcataaggagttcaatgagagttgttgaccaaacagtgagtttcattatctgaacatttaaaaaagttaatgatatttttaaaatattggttttattttcaaatatgttttgaaaaattcaaactttgatctcaatatgataaaatgttaattaactcagttaaattagtaaatcttacgtaggaaaagtgaggttgttattaagctagtgtggggggaaaaggagagtacattattatttttatttaatttttttaatctgtataaaataatcttaagatgataattattttaataggtaggtatttctaagttttacaagtccaatacttcagcctaccaattacataggtcaatacggtccaagcagagtacattcctcactgccaatgcccgtgtacagaacaccaggctctggctgacagatttgtttagggctctcttttagcccaattcaaaaagaaaagggagaaaactcacttgggcttcatcatatacatgataattcagccaattatctgttactaacttcatcacctcattaattaagcctaacactcatctgtttaattaatttgttcttataattttttttcacttatatttgatatcgaaataatatctaataagaacatgaaagatgagaataattaatctgtaaatacaaaatacattaattaagacgtatgttcatcttatatatacttttatctttaacttgatgcacacgtacacattctatgaactcttcacgttcaaaatagatcactttttttttctaacaataaaggatcctcacaacaagaatctataagactaattccttgaaaaagtacagtatcggtttttcacatgtgatactctatttacatattcattcataaatcgaaaccctaatatatacttaagaattcaagttatttgtcacttatgtcatttattgtgggtagattactcctgtatatgtgcatgtgcgcaacttttcacctttttttggaaaagggagtagtgttattttggttagagatggtattgaaaataatatacagcaaaggacatggacacatggtaataatagtgatatagaggatggtgtccttggaaattgtccgcaaaacaacacaaggcaaatgacatggcaaaagagagg

SEQ ID NO: 35 - protein, 267 aa, Glycine max
MHKFVEIARSNKMKMKVLVLFVATVLVAYECHGSDYQKFPLQMKTGFGGQYSNEVACASWRLGVEANNVVKWQTVPAACGEYIADYVLGDQYRSDSKTVNQQAYFYAKSLKLTNKDVFVLDVDDTTLSNLQYFANHGFGVEPHNTTAFKNWVLDGEAFALPETLKMYNKLLALGIKIVFLSERPLSLGDVTAKNLKEVGFNTWEKLILRDPSEYSGKLSFEYKSAEREKLEKEGYRIIGNVGDQWSDLLGSNKGTRTFKLPNPLYYD

SEQ ID NO: 36 - DNA, 10722 bp, Glycine max
  source 1..10722: mol_type = genomic DNA
gggaatccaacagaggcctaccctttcaatgtgaatggctctcctttaggggtggcagctatttgttccccagatgggaggcatcttgccatgatgcctcatcctgagcgttgcttcttaatgtggcagttcccatggtatccaaagcagtgggatgtggagaagaaggggcctagtccttggttacgcatgttccagaatgcaagagagtggtgttcctgaaatgatcaaatgtttttctcatcttgtcttatctttttcttttctttttccatataaagcacttgtccttgctttagttgacaacttatccatgtcctccttcaataattaatggacaggttactcatcaataacaaatagctagagtgaaattacaagttttaaggaattttatctaatgcttgtttgctattcaacaattcttttatctgcctattgcctgatctgtctaaatgctagccgagcactgtctttaacgcttaattttcttaatagctgaatggttaagttgctttgttaattttttcaatttcactgtcattttgtgtctggtaattaagtttcttggaaatgtgagttgaatttattagaatatttatgtgcctaccacatgtttgatcatttactggtttttcttttacttttactcattagtttatgcttggttattttacttgtttcaagcatgactgaaagaattataagtgtaccaagaaaggaacgcttgtagcttgcaatgaagcatggagcttgaagcacttcccgatgagtatattcattatgctacaaagaagtgctcatctatgccaaaagaataccattccgtgtttgagacaatcacaggtctggttctcgtgaattttttgtccttctgggagttatgtagattagtccaggatgattaagacatgcttttagggattaagttttctatattataagttagcaatctagtaatgcatgattttaaagtagtttcacacttcaagataatgatttggcatatgtttattacttgggattactggatttgtgtttttggtgaattttacttctggatgggcatggatatggtaactgtgtcaggtggttaatggtgagctatgcttgattgattatttcaatatactgatgccattgtccagctttgatttttcaacctgagattatttttatcttgagtgctatatattttcaacatgctgatggtactctgtatttctttttgcttcatggttctttcatcatgttaggtaagatatatctctaattcaacaaatgcaagtctgatgcttgtaccttgttgtttatagagaaatatgatttaataggcggatatttctggacttcaaatatgaccccatccaagtttatgtcattgaggaggtagagatgtgaattgggttgtagcaggtacaattgagctgtaatgtggtattccattttaagagttgataatagtttgtttgttttttctttgatgattaatattggcataaatgagaatttttttaagaaagttgcttgtgccctgtgaaccttattgctactaaggcctactagtaaagagttaataaaaaactatattggtcatacttaatgaatacacagatacataaaacatataaattcaattggttctggcaatagtggtacatgcataattcaattcaacagggccctgtgaacctgccctgactttttcatctttcttcactcactcagtcaccaatgccatttgcatcactctaagcattaattccttctgtatccatatttgagtgtggtacgcagttttgttccctttttttttatcatgaagccttagcgtttgtattttatttgtaagttcacatgctttaaatttcctttcctattataagttcaattaaaatgccgtggtggtttaagaagcatagtattgtgatcactttatgcctactatagtaacttgaaccctatcccatcaaatggccaaagagactttaatgcaattattgcttcgattcactcctcacatgcttgctagcattcagaaaattcatctcataattttttggtctgatactgttagttggcctattttggttttagttaatactcatttctccaagtttagataatttaagttttctttcctaaagttctaatattatttgaactaaaatgctaagtttcccatgttcatcttttagttcctattttcttcgtttaacttgagcaaaagctatgactggcacatattctttggttgttatcatgcataggcattttgctgcaaatctgttctctgtcataaaattcaacagttagcggacaatgtcgtgtgaagcttcatcatattcacaactattgatgctctgattaagcaggaaatattccttagtctaattggtattatatatacggtgttggctgtaactgtttctagtgatggcgacttctaatgagttatcttatcaattttcagtacccataatcccatattacagttagaatcactttttataaatgaatattgtaaagatatttattgtattcatgtgtaggtttgagtacttgtttccaagttatttgaattaatccatgtatgcataatgcatagatgaaggtctgcgtcttattaagagaattgaaattcatgaaaatttcaccacccatatgtggtagtggtatattctgataagatgtatactacagctagctagtcccatagatagcacagattaagagagttaggattttgaaagaataaaaccaaatccaaactgaattgccctactctagtgagatgattagaggacaaccaagataaacctagaggacaattgctttatgcacccataagaaacaattgagaccccaaatggaagtatatgcccttctcttcctatactgcacccttcctctctctcagccaaccctagttgtctccccccaaaacctccataacaatgacgccccacctccgcccaacaccacctccgcccaacaccaccgccgcacccaaatatctttgcaatcactcattgccacctccacaccacaccaaaccaaccaccatgctgctacccaccaggccaccactgccatgtgcaaaccacccaccatcaaaattgtattatgaaacacttgatttgtaatacacaagtattccaaagtaaatgttatgttccgtaataaatcaacatatattacaaaacatgtatttcaaaatacataacgcttaaaccgtaatatacaagtatttcaaaaatatattctatttcgtaatatatattgatacattatgaaacatgtattttgaaatgcatatttagaatacctatgtattacggaacactaacacataacacatgtattctatctatttttccttttttataatttaactagctcttttagatgggtcataaaatttacttaataagtagaccaaaaaaatacataattccaaagtatcaattaagataaatgtttttttttacggaattatgattaatttttaatatactattgttattattatttacataatataaattgatgtattagatctaaaaaagttcttcaaataatctaatgtatgtctttttaactaaatatttttttaataagtcttaacttattggttatgtctcgtccaagtttgataaaagctataagccttagaactccgatgaatgcttgaccaatttattcctcaggcaacagaggaatttaacaacttaaaaaatactactcaatcctcccgttgagctatagaacggcatggtgcaatctttatataaaagcgccaccaattagatgttgtctcacttgtgtggacttcgattatctgtggtcataatccaaggactcaatttaattctttttttatatttcattaatgttgtaaggttaatttggaaaataaaacaaagggaagacattcaacttcccctctctctagctgcagttaaaccacatgtgagccttaagatgtacacgtgtcaagctataagtaagacatttctttataatgaaaaatacaaaaataaaaaaaaaataaaaattctaagcaatattgagtttctttgattaagatctctggatggatcagatagcaaacttggttttgactggtttggcaaaatgaagaaaaacaacgaaagaaagatgaatttatgtttattttaaatataaagtttttggacgcaaaagtgaaaattttaaacattcttatatacagatatataaataccaaactgaattgatgaagtaatctctttttttttttgggttattctgaagcttaacactgaagaaaactggtgaagttgagaatttcctttaattgcagtatggagcatgtttgtgttcctagagccacaagcaacacgtagaagagacaaggagagagccagaggaagaggcagagagagtaaaagaacaaggagcttgttatcatcgtagggtccatgaatgtaattaataggactcaatcaccttcatcaaattataatccgactcaccaaaattaatttcttgtatctacccaataataattgagaaacaaatcttatattaataatataaaataattttacatattattgaatcataaattatcttaaaattaaataatgttataaaattattttacgttctaagtggcattaaactcataataattcatgttaaatttatcttcatctgaaaatactctaagattctaagttaacataatttcatgaaaattgaaaattggatatgttggctactatgtggggtgaacagtaccatctttacgtaaataaaacaccacctcaaatattatggtagttgttctttaaaaaatactgataatattatcattggttatcaagattgaagagcactgaaatgacaataaccaatgctttgaatttgaataaacgacaaaagttctcctcacaggcttgtgtgtgtggcaataacatctccttaatactggtggagatagaaaaagcttgaataccaaccgcaaagagtaggactaagagtgacaaaaaaccttgcaggcacattatgttgccacgtgtcggctgatcaatcttctatgtgtggatgtaaggcttctatgaccaatccttttgtgtacactcactccaccaagcaactactataagtcacagcctcattcatggtcacaagccacttagcactgagtcttgcaattgaagttgaagtcttgcccttctctcttcaactccaaacacaagacatcatcatccatggctgcctccgtctccactgtcggagctgtcaacagagctcttgtatgatgcctttttcccactttaatttcttacactttgcacttttttaattactattaccctcgttaaggtcttatttcatatgcatgcttttcatgcatgtattgtactcccctacttttttctctttacatgtatgtcatatgatatctttaattaaattacaatcatgattttttttcctttaatttcctttatacttaattttccacttagtcattattgatcatgaactggaaagtttcaatgatgactaagtgaaaaacgagttggctcaaatgtatgaatatcttccacatgtttatatacacctcactagaaataatgtatcatttccaaatatattcattttacgtgtcatgtatagtcacatgctaagtttaaatggattaattggtgctattaggctgttatgaagtggcttgatgtgcatgtttatatatatatatatatataaaatcttggaaatagacaaaaaaataattaaggagaagaaagagcaactatttgaagtactgaatatttttattttactgtgggattgcagttgaacctgaatgggtctggacctggggtttcagctcccagttcatccttctttgggagcagcttgaagaaggttattggctcaagggtccccaacacaaagatttcctctggaagcttcaagattgttgctgtagaagagaagaaagagattgaagagacccagcagaccgacaaggacagatggaagggtcttgcctatgatatctcagacgaccaacaggacatcacaagaggaaagggtttggttgactcccttttccaagctccacaggatgctggaactcactatgctgtcatgagctcctacgagtaccttagcactggacttcgccagtaagtactcactactcacatatttacatgtttaggatcagtttgtataaactttaaataagtcattttgaaaaaaattcttgtttaataaacagacaaaatttacttatgaaaataaaccaaaaaaaactgttttttttttctaaataaaaacgagttagtcaaatatactaaaaaaacggtttatttttattaaaataaatatatttttttcaattaataagtttaaacaaactgtaatttagtgttgtttacttcagttttgttatgaacctgacccttatgattgatggtgtttaaattaggtacttggacaacaacatggatggattttacattgctccagcttttatggacaaacttgttgttcacattaccaagaacttcatgaccctgcccaacatcaaggcatgccactacaaacatattaattctttgcataaactgaacagttagacttttgcaaaattgggagacacattcatttttttttcttcaatattttaccaggttcctctcattcttggtatctggggaggcaagggacaaggaaaatctttccaatgtgagcttgtctttgccaagatgggaatcaagtgagtttcttgctgaactagcccctccattatgtgcatttttcaaaaattcagttagatcatccaaaaccacattgaaatatcagcaaacataattttaaatataaaatcacttgttcagtcttatgttgaacaattattatgagtccaaaattgattttgactttaaacaactttaactttaagtaacttttgtattggatccaaaattttatacagtttaacggttaaatttctaatttcaaaataaaaaggtccaaacatgaaccatttcactttaaaaaaaatcatctttataaaacctcagaaacacacacaaaagaaagaactgatgtcttgctactatactcatgctgcatcaaagatttgaagtatattgaacctcaagtaaaaaaatcaaacactcttttaacatttaaacccctattgtggtgctgtgattccattgcagccccatcatgatgagtgctggagagttggaaagtggaaatgcaggagagccagcaaaactgatcaggcagagataccgtgaagccgcagacttgatcaagaagggaaagatgtgtgctctcttcatcaacgatcttgatgcaggagctggtcgtcttggtggaaccacccaatacactgtcaacaaccagatggtgaatgccaccctcatgaacattgctgataaccccaccaatgtgcagcttcctggtatgtataacaaggaagagaatccccgtgtgcccatcatcgtcaccggtaacgatttctccacactctatgcgcctctcatccgtgatgggcgtatggagaagttctactgggcaccaacaagggatgaccgcattggtgtctgcactggaatttttcgcaccgattcaattcctgaacaggacgttgtcaagattgttgacaccttccctggccaatccattggtaagaaccaagtacaatttggtggttacatgtgttaaaatggaacaaaacataatatatcaattctattctatgtttgatgtgttctagaaaatagaaccattagaaatctagcattgactagcgtatataagtagaagacaatcctcaaattctaacatatagtatcaaacaccttgtaagatgtttagaaaccacatataccttaaaaatcatagcaacagtgaattctgattgatggagctttactctgatagtgtatagccattaaattcttacaataatagtatcgccattgtttttcactttggatcttattttcaaatatttgtatagaacgagtgaaaacaagaatgactaaattaatagtgaatactaatgttatcccatcccatgatgttgttgtacaagtgcagatttctttggtgcactgagggccagagtatatgatgacgaagtgaggaagtggatttctggtgttggtgttgacggcattgggaagaagcttgtgaactcaaaagatggacctccaacctttgagcagcccaagatgactctggagaagctcttgacgtatggtaacatgcttgtccaagaacaagagaatgtcaagagagtccaattggctgacaagtacttgaacgaggctgcacttggaaatgctaacgaggatgctattaacaggggaactttcttccaatcttgaattggaggtttgtataatgtttctctaacttgcaaaaataatgttaggttcaaagtataacttggtctaaaaatgttgaatttcttttttttttttttctgtgcaggcaaagcaaacttggcaagtaaatatttctgttcctgaaggttgtgctgatccaagtgctagaagttgtgatgggacttgcgtgtaacattcttagaattctcaatgtcttaggtgcaaagagaagatgccaagtttgagatgattttactatatttttcatattggactgtcttcctctataattttgagttaaagtgtccacttaaattgaatttggtgccttttccttttgtcctaaattcttgattttttatgctttaaatttatatcttttgtttgacgtacaaatattagtacatacgtagcgttatatcttcattctcgaagtgctaaggatccgaagaacttggagagaaaggtcgagttcagagcttaataatgaatggcaagctgccaccatatcataaacaccacaatcataatcattaaaaattgtttcttatatacaaggataaatagtcacttttgtccctcaatatataatttgctgacaaatacactcttgaaagatgaaaatacaaaatttagtctctgaaagtgtaaaaaatgtgacaaatatatcataccgttaatttttgtctgtcatcgttaataaagtcgcatacgtggcacaaaatgataaatttgtcactgaaataatagccaacatgaattgccagcatagaggcatatttgtcataatattttttgacttttcatcttttcactttgctaacagatgcatccttaaaagataacaatacaaaatttagtcctcgaaaatataaaagtgtcacaaatatatccgaatgttaataatagattgtgattaattattattattattattattattattattattattatgtgtttataatttaatgctcatatttgtttagtacttatgtaatatattttttaaattgtcttttaatatatatatttttattattttaattttcttgtcaaaccttaatctttacaattaaaagaaactttatcttatatcttataattttatcaaatttctactaaatttttcatttttaatctttaaaatgattacatatcccaatttcaacttaagtaaccttatatttagattgaaaataatatgtcgagagttttgagtagtaaaaacacaattgactgcaagatcaaatttatttatttttgaaaaaaaatttaatcaactatttttttttttaaaaaaaaagtctcacaaaatttaaactagataaaactaaagtggaattttttttataaaaagtctcacaaaatttaatcaattgagttatcatttaaaaaaataaaagtcttttatttcttgaatttttttgtttcttcaattaattattaatttttattatgtactcttccataatatgtcaaatggaatgcaaggtcgttgcagcttctaaatcagcgagagatacaactctacgttccgcaagtaataaacctatgtactagtttttttttgaagttaacctctgtattagtttgagcattattgtttttttttatttaaatttctttgagttctttatttgttagtaattatttttcatttcattcgtattatgtataataagtgttgtctcgagtggaagcacataagatttatctacgagattctttcatatttgagatgtatatatacattttattgattaaggaaaaagacttataatttcacttcagttcaatctagtttaaattttctgagacttttaaaaaaaatagttgattatattattttttaaaaacaaataaatttggtcatttggttcgttgtgttttttctactcgaaatacttgacatattaatttcaatctaaatattagagtacttaagttggaattggaatatagaataattttaaagattaaaagtgacaaatttagtagaaatttgataaaattataaaatataagataaagttttttttaacagtaaaaatgtttgacaaggaaatcaaaataataataatatatatattaaaagacaattcaaaaatatattacataagtactaaacgaatagtagtaaattacaaacaaacaaataataataataataataataataataataataagttacaatttattattaacatccagatatatttgtcatactttttatacttgtagaattttgtattttcatctttcaaggacgcacttgtcagcggagtcgaaaagttaaaagaatatt

SEQ ID NO: 37 - protein, 443 aa, Glycine max
MAASVSTVGAVNRALLNLNGSGPGVSAPSSSFFGSSLKKVIGSRVPNTKISSGSFKIVAVEEKKEIEETQQTDKDRWKGLAYDISDDQQDITRGKGLVDSLFQAPQDAGTHYAVMSSYEYLSTGLRQYLDNNMDGFYIAPAFMDKLVVHITKNFMTLPNIKVPLILGIWGGKGQGKSFQCELVFAKMGINPIMMSAGELESGNAGEPAKLIRQRYREAADLIKKGKMCALFINDLDAGAGRLGGTTQYTVNNQMVNATLMNIADNPTNVQLPGMYNKEENPRVPIIVTGNDFSTLYAPLIRDGRMEKFYWAPTRDDRIGVCTGIFRTDSIPEQDVVKIVDTFPGQSIDFFGALRARVYDDEVRKWISGVGVDGIGKKLVNSKDGPPTFEQPKMTLEKLLTYGNMLVQEQENVKRVQLADKYLNEAALGNANEDAINRGTFFQS

SEQ ID NO: 38 - DNA, 10957 bp, Glycine max
  source 1..10957: mol_type = genomic DNA
aatttaatatgatatcagagttgatcacaagcatccttggaggaggatgttaaaaaatattctaaagatgttatagatagatctgttggaccacttaatgccatttgccgctctaaagtctaaaccatacaataatttctggctaaagggggaggttaatgtagaaatgaagcagggtgcaagaacgaagaacgaattgggagacaatgccattaacacttgcaaaactttctggtttctgtcacttcaaaactgtgaactattttgtctttgacccattggaacatgtgtgagaagggagagctatgagttggtggatgaaaaacttcgggctttcgagtataacgatgaaattgtctttctctatcatgtatcaatcatcgttgttatctcttgactgctaacttctaacttttttctcaagttgcaatctcatccagtcttaataatagaacactttttagcatttttttttacacactttttatttttgttaaaattcattaaaagttaaaaaattaaaaaaattccattaaataaaaagtgaaacacaaaaacttataatttttaataaattttgacaaacaataaagagtgtatttaaaaggatgtatcactaacatttctttaaatataaatcttgagtaacgaatcaaattcgttattaaaattataaaatttaattaacttatttcttgaaattattaaacacaaattattttagtctttattgccataattttaatggttttaaaatttatgaaaattcgtagactttatctacttaataaaaaatcttaatttaatgattggattaataaaatttaatatatatatatatatatatatataaaagtatccaaggaaacactagtttttaccaagtgttattcaaagaaaaacttgatttctctcctataaattttgataaaattaattttatcaacagtgatatgatattgttaattttttttaaaagagctaggttaaaagtggatttgtgtttagaaatgaaaaaattaaaattaagattagagaatgaatactactttggtgtaatttgttttataaaaattatagtttatttttattagttttatgagatgattttttaaaactaagccaattatttaaaacaatcttacaaaaaaattacttaattttatttggatgttagaatcagattatttataacatttaaatattaaagatacttatgaaaatgaatgagttttttttttaatacaaccacacatataatctatttatgtacaaagagaaattttatgcaaatttgataggatcatgatgagcagccaaactcaccatatgaatatttgaagcagcaaccgcatattgaaagttcaaatttggtatatatgctgttaatgctcattttaaggttaacatgaacattggtttttgttttaatttctagcacttatttatagctgtgattgataaactatatattttatttctataattattgttatttattatttattattttataatacaaatgacaatctacattatttttattttagtttattgtgattaaaaattaatttgttatgtaccaagatttttttggatattttttattttatttttataaaaatgacccttgaattttcctatatatttggcgcacaacttgccaagggttgagttgtgctagcaaaattttaatctatcattaaatcagttcaatataatccaaccgacaaatatcttaagtcgtgaggagtcacttcaaatggataaaattaatcaagttgacatttctagatgaaataaccatcaatacatattttttctcttaattttactgtcttaaaaaaatctcaatatcaattttttttggtaaatactttcaatatcattttgtttattcattgcacaactaattttaaaaataaaaagttaatcaaacacaacttgttagtgtgctctttaatctttaacaacacaattttgggatgtaagtaacttatcaattagattaaaataaaatattataaaaataaatttgaacagtttaatatgggatgcttacatgagttacttaactttaaacaaaattatttattatttttttattttttctattggaacatatggaataagtttgttgcttaacaaatttatatcttaaataacatatttttaaaatattataggacccacaaattaagttaaacattcatcttaataatctcaacattaaaataatgaatttttgtataaaatacttaaaattacatggaatctacaaaattaagataaaaacacatctaatcaactatgcattgtagatgctatgagtaatagggaagaaaaaaaagattttgtcattttattgttaaatccttattgaacgtttagaagactttataaaaaaatgaaaaggctatttataacttttacaagttatttttattttaataaattcttttacataatttattaaaacaatatttttaaataaattttataattaagtttatgaattaagctttatgcaataaaaaattattaaataaatatttaattaacttgtttgtctaaatactccacgatctattacgtttctcacgagccaagagttcgatgatcaatgtttttttttttttttatgtttaactcttcttttaaagttgtttatttttaattttgttttctcgattttctgctaagagttttttttttctttacacaggtttaatgtagtctccaactttaattacttttttttaacattcaatgaggatcgagcttcaacatttataaattaatagtaaatatttttgtgaaatttgttatttactaaataattatatattaaaaagttaatatttaaataattgtgtaaaaagaatcagtccactttaacgcatctttggatctgttccaatctaaaaatatgtttgaaaataaaatcaattcaagattatgcttaattatttgaaagactttcggacacttttgcaaacacaatttacaaacataggtttaacccaaaattgagttaaaaaattttaagtcaaacatgcacttaaacatattgattttaatttaaatccaacctgatctaataaaaaataatttgaatttgttttgtatttttaaaaatactaaatatagttaaaaataatgatttttttattatcttgtcgttagtaaaaaaattgattaactttgtcaataattaatattcacaaaatacttaatcgactatctttaataagatctcttaaatcataaaactcagatagtgagaccgagtccgaatacattatcactagaactaaggatatgctggtgttgaaaataatgaatttgataaactagaagtaaattaacaatattaaaatttaatttcacaagctaattcctaaaatattatttataattaaagatgataaaataaatatagtttagaatattatttttgtccttctcattcaccatatcatcacaaaataaggtgggatcggttagtttattccaaaaatagattgaaaaggtcaacttagcttctttttttttttaaccattcacgggccgatggatcatgaatgtacatgaaaataggaatgaaataaataagaaatgtttaaattaaaaaggaatatgcatggtggagtgaaggtatgagaccactcatatttttgaatttttttttctctcgcctatttttttaccatcaaactcacctttagataaagatagtcttatgctcaatttaaaagaccaaaacttatgaaaaaaaatgctttggtgaaaatggccgttagtaaaataaaatgaattgaaaattaagaattctaaatttttgagggagaatcacggggcaccagcctcggtctgtgggttgagatagagcatgttgcgagacctatgaattggatattttttaattcacctttttttttcctttcataatcagtacttagactattatctacatgaatttatttatcacttatttaagaaattaataaccctcgactttcttagtaagagttcatttgaattcactagtgttttaaaataaactcttacttttttttttataaaaaaaatatattctaaactgcaaatacaaaggctagttagggattaaataaatcactgctgttaatttgatctaaaatacaaacagaataaacaagttatgttacaaaatgtcatcttatgatagcaggtaatttggaaggactagctctggaaccttcaattatagcatgggcatgcttggagcacataaaggatgtatcacttaaccaataagacagggagaggcacatataaaagctgaaaaaatcaccttgtgtgcatcctttggcccccctttgaaccgggagtggaggggggtcattacatcatctcaatcaaccactatccttgtttctattaatgcaatggcccactttttgacataatttcataattttttttcaacatcattggctacttgtgtgggttagcatgctaaaaatctaaaataaatggcagcccagccacagatttttatgtaattgaccaatacaaatattagaaaaaaaattatgcagtaaaaaattttacattttcatttaattaactttattaacttttatgataattacttaaaaaattatattaagaacgaattatgatttacataatgtaaaagtgtgtaacaattaaattctagaaatattatagtgttgttgctgaacaattttctcttctagtgaaagcaacactagccaatgtttgatcctgatgagatgaatgctacaagctacgtgtggtaacaaattctccttgaaacttatggatcagaaaaaggcttcaattccaacctcaagatctatggatgtagtaggttggtgagacctggaatgcactatgtagttgccacgtggcagccaagctaatcttcatcgtgtggatgttaaggattctacaactagttttgaggatatgaatttctgaacactcaattgcaccactgccataagtcccttccctattgtaatttctgccagcctcaaaaaagacttgctctgcctctgccactctctgagtaggactacactgccatggctgcctcagtctccactgttggagctgtcaacggagctccagtatgatgttttctccactcttttcactttcttgtccttagtttctcaactaattcttatatgaattttgttatataaatgttcaaatcactattagtatgtgtaaggttatagaattatgtttgaaatatacatataacatatctatacttgtcaaataatggagttgcactgtggagtaaaatcaattaacttttcacgttgaaagtgaagcaactcatcatgggatttctacgttcttttatgtagtttaggtagccaatgtgcatccaattccaaacacgattttgaatgtccgaaagatatatgacactacctttatcctagtttagttgagaattattccaatatacatgtctatgacaagtttctttttcgtgctagtaagaatcactgagttttggtacacttctaaaagtgtaaatttaaagaataagatacctagagagaaaactctagtgacttttaactcctcatttatattgatgtaaactcattataggttagtaactatgtgttatgtaatgtaatagtcgatatatgtgatattaaattagaagacaatggtgttatttcttacttgagtgaagataaaaacagatttggatattagaagcatgtgacaaagtgagtgtgaagttttaaaattagttgtttgaacagtgtgaaagagtggttactgtgaatattgcagctgagcttgaacagctctggagctggagcttcagttcccagttcagccttctttggcaccagcttgaagaaggttattgcctcaagggtccctaacgtcaagatttcttctggaagtttcaaaatcgttgccgcggaaaaagagattgatgagaaacaacagacagacaaggacagatggaaaggtcttgcctatgatgtttcagacgaccagcaagacatcacaagagggaagggtttggttgattccctcttccaagctccacaggatactggaactcactatgcaatcatgagctcttatgagtaccttagcactggacttaaacagtgagtgacacatacaaaaatagtgcttgtttgtttattttctgtaggaatcgaagacagtatcaaggggtttataataactcactatcctattcaatcaactgagttagaccccttaactagtgtatgcttattatcacttctattgtcatgaattgaaaaactaaactcccttatatttttatctaactagaggtgttcatgatggtggtatttcaggtacaacttggataacaacatggacggtttttacattgctcctgcttttatggacaagcttgttgttcacatcagcaagaacttcatgaccctgcccaacatcaaggcatgtaaattgcacacatgattccttcatacattgccccaagactttagttaatgaaacaggacacactcagtatttttcattgttttttacaggttcctctcattcttggtatctggggaggcaaaggtcaaggaaaatccttccaatgcgagcttgtctttgccaagatgggaatcaagtgagtaaatgctgtatatatattactactacaaaacagaacacatcagttatatacattactacattagcaactctattgcattagaacttagtacctagcactcagtagttgaagtgtgttccaatgacatctctaaatggaactttagttgaacgtaggattaaaatatgtttttgatctctcaaaaatcgagtttaatccctataaaagttcctatatttttacataaatgcatgtgtagtctctggtgagagactaaaactatgttactcttaataaatgtattttagccttgaatatatcatttggtaattggtgctaatttctggtcctcatgatgatgtgattacagccctatcatgatgagtgctggagagttggaaagtggaaatgcaggagagccagcaaaactgatcagacagagataccgtgaagccgcagacatgatcaagaagggaaagatgtgtgctctcttcatcaacgatcttgatgcaggagctggtcgtcttggtggaactacacaatacactgtcaacaaccagatggtgaatgccaccctcatgaacattgctgataaccccaccaatgtgcagcttcctggtatgtacaacaaggaagagaacccccgtgtgcccatcatcgtcaccggtaacgatttctcaacactgtatgctcctctcatccgtgatgggcgtatggagaagttctactgggcacctacaagggacgatcgtgttggcgtctgcaatggaattttccgcactgacaatgttcctaaggatgacattgtcaagcttgttgacaccttccccggccaatctattggtaagtttcctattgggaactcaagaatcctttgctttgcaaatgatctatatgtttcacaccccaaacatttgggtaactcattgatatttaatacaaacttttattatgcagattagcagggtgaagctccaattatgattaaaaaacagcatgaaaaaacatttaagaaactacatttaagttttgtccatgtaattaaactttcatatttgtcccttaaattataagcaacaatcactttaatcctgattttttttaagggtaataatatgtggacacctttacagactatttctctttatatctcttatcctatcacatcatatatctcagctatcatgcctacacttttctctaggtgtcatttagctcttagatgtccttacattacaataagactaatggtatgcaatttatgattgcagatttctttggtgcactcagggctagagtatatgatgatgaggtgaggaagtggatttctgttgttggtgttgacttcattgggaagaagcttgtgaactccaaggaaggacctccaacctttgaccaaccgaagatgactttgagcaagctcttggagtatggtaacatgcttgtccaagaacaagagaatgtgaagagagtacaactggcagacaagtacttgaaagaggctgctcttggtgatgctaatcaagattccatcaacagaggaactttctatggtttgttcttgtatcctgaaccttgatataggatatttgcatatagcttaattttctgtcaacttctacacacagacatttaaattttgatttagtaaaaaggtttccaatatatctaataatatattgctcacttaaactattttaacaatttgacaaaatataattgaaagattacacttcttcaaaattaaactcattagaaagtgtattttgataaaataaaaccaggtctaaactttttggttgttacattctctgcacaggcaaagcagctcagcaagtaaacattcccgttcctgaaggttgtactgatccaaatgcctcaaacttcgacccaactgcaagaagtgatgatggaacctgcttatacacaccctgagaacctaagcattctccacttggagagaagctaagagttgagataataaaataagttatcttgttcatattggattgttatcctgtataattttgagttacattgtctacattttacaatgtgtgcaatgttaaagtcttgatgtctttctctttccgcttgcgcatttgttgcttttctacatttaattttttccttgacacgatattaatgttatgctacctgttcgttttagttaataaagtttattttttgtaatttgtaacattttgttagcattttttttaaacattacttcctgtaacattatttaaactcaatttttttttattctcactattatatcatatttttttaatgccgttttacatttctcatttaattacttttactttgatctagtatgatcaaaatatccttcatttgaaatatattcttggtttctaattttattagaagagaaagtaaaagataaaatgaaactgaaccacacgttttcttaattaaataagtatgaaattcatcaaaagaaaattaacatattaagaaaacaagatagaattctcttcttggggttattcttgagctaacatattggcaacaaacttcaactgcagttggaattgcaactgtgttgcggccattacgggcaaatacaagttaattgttacataattcgtaatcacagcataatgactgctaacgcaatttaaatattcaagcggcatcagatgatgagatgacgtgatccacaatttaaggtccattaatcagccatataaacaatttagtgactaatgtaaaaccgcgagaatgttcattttggagtcacagtgtgaatcagtgaatgtgaatgacttataatatgttttgtgaatcaaatcaaaattgatattttaagggtgtacaaatatgtaagaattgttatccttgtaactatatttgcggtggataacattctcgttatgattaattgtggactccgaaaagctaagattaataatgtctttgattggcttgttcagcagagcacatgaccgaagctgtggtctgttgtttccctgaaccaactgatgagagccacgtagaattggcggctattatcgtgggtcggtgtttcggaggagagaatttaatggtgttttaatcatcagaagaaaatctgttgaactgcttctagtgctaaatattcagaatatgccgcgtttcacgtggaatctaagaaattagacacaaagtagcgaaactatctaaggttgttcaatctggctagaaactataaccataataaaacacatgttaatgacatataaatatcttttaattttaaacattctataacatcttttaattttctttttctgtcatattataatatatttttatcttatttaagacatgcatgagtacttatatttaaacactcataaattataaatacttcatgaacactattccttttttcatccctcttcctgatacattacattatcaattaaatcaatttactcaggattactcaggagaggagaggatgttcaagaatcatttttcaaaccataaccggatattaatcatagtattatatgaaaacatgatcggaagtctcgattgaatgaatccaaaaatagcgaagcatcattatcattattaagtggtgtcaacctaactcaattacatctgctcacgtgaaagccaagaaccacgtttttcctattccattccatacggcacatattacccttctctttatatttcattttctcattcaaacaaaaaatcccaacattctagtcccgtcaagcatacggagaccaatcccctaacacttccattccatatttcgaacgttttctctttctccaccgctttcgcgttggcaataacaacccgcgaacccaaacaaggtggccacggatctcgaatgtaatattatgttagattttatcttaaaaacaattggtattaagttagattttatcttaaaatcaattggtattaagtgaaattattcaataaatatataaactgcacttcaaaaaatgagaccgatatgagacttgaatatttctcaatatatagattataccacattgacgacaacaacgtgggcgttaacgtcaacagcgcggttgctaacaagtctgtcaccgctgctcacttcaccgacgggtcatccaagcaaaacctcagtttgaagtccggcaaagtgattcaggcgtgggtggactatgattcctcgaagaaacaattacatgttagactttccttaacctcgtcgtaacccagttcaccaatcttgttttgccaactcgatctctcacttatttttcatgaaaacatgtatgctgggttttcagcttcaac

SEQ ID NO: 39 - protein, 478 aa, Glycine max
MAASVSTVGAVNGAPLSLNSSGAGASVPSSAFFGTSLKKVIASRVPNVKISSGSFKIVAAEKEIDEKQQTDKDRWKGLAYDVSDDQQDITRGKGLVDSLFQAPQDTGTHYAIMSSYEYLSTGLKQYNLDNNMDGFYIAPAFMDKLVVHISKNFMTLPNIKVPLILGIWGGKGQGKSFQCELVFAKMGINPIMMSAGELESGNAGEPAKLIRQRYREAADMIKKGKMCALFINDLDAGAGRLGGTTQYTVNNQMVNATLMNIADNPTNVQLPGMYNKEENPRVPIIVTGNDFSTLYAPLIRDGRMEKFYWAPTRDDRVGVCNGIFRTDNVPKDDIVKLVDTFPGQSIDFFGALRARVYDDEVRKWISVVGVDFIGKKLVNSKEGPPTFDQPKMTLSKLLEYGNMLVQEQENVKRVQLADKYLKEAALGDANQDSINRGTFYGKAAQQVNIPVPEGCTDPNASNFDPTARSDDGTCLYTP

SEQ ID NO: 40 - DNA, 11400 bp, Glycine max
  source 1..11400: mol_type = genomic DNA
taaaaatggattttttttagaaatgaaaattttaaaattaagattagagaatgaatgctgctgtggtgtaatttgttttataaaaaaatagttcatttttattagttgtatgagataatttttaaaaataaaccaattattttaaaaaatattactaaaatttttattttatttggatgttagaatcgaattatttataaaatttaaacatatattaaaggtatttataaaaatgagtgattttttttatacaaccacacatatataatctatttatgtacaaagggaaattttatgcaaatatgataggatcatgatgagcagccaaactcatcatatgaatatttgaagcagcaatctcatattgaaaattcaaatttgataatgtgttgttaatgttcattttaaggttaatacgcacattgatttttgtttttaatttctagcacttatttattgctatgattgatacaatatactttttgttattttataatacaaattacaacctatattatttttattttagtttattgtattttaaaattaatttgtcatgtaaaagaattttttagattttttaaatattttaaaaaagaatgacccttgaattttcctatatggaacacaacttgctaagggttgagttgtgctagcaaaattttaatctaactaaccaaattgacatttctagatggaacaatataatctttaatttttctcataatgttactgtcttaaactaaaaattcttacctctttcaatatcaatttgtttactcatttcacaactaattttaaaaataaaaagttaatcaaacatgtgctcgttaatctttaatgaccgcacaacgcaattttgggatgtaagcaacatcttatcaatgattagattaaaataaagtattataaaaataaatttggacggtttaacattattccgttaagtggtgggggaagaagaaaaagattttgtcattttattgttaaatccttattggacgtttagaatactttataaaaaaatgtaaaggtaatttataatttttacaagttcttttttattttaataaattcttttacataatttattaaaacaatatttttaaataaattttataattaagtttatgaattaagttttatgcaaaaaaaaaattatcaaataaatatttaattgagttgtttgtctaaatactcctcgatctatcacgtttctaacgggccaagagttcgacgatcaatgcattttatgttcaactcttcttttgaacttgtttatttttaattttgttttctcgattttttgcaaagagttttttttaacatggaaaaagtaaattaaaaaattattaataggagaaaataataagaaaatagttgttgatttggtaattttttaacgacaatagatcttatttttatttttatacataaaattaattaatagtagatatttttgtgaaatttattatttactaaataattatttattaaaatttaatatttaaataattatgtaaaaaaaaatagtcccctttaatgcatctttggatttgtttcggtcttgatagtaaaatcattttaaaattatgcgtagttatttttcagaaatatatttgaaagtcaaatttcatctgaaaactcatattcaaagaagtaaaacttgagacacttttacaaactcaatttacaaacatatgtttaactcaaaattaggtttggaaacttgaagtgaagcatgtgcttaaacatattgattttaatttaaatccaacctattctaataaaaaataatttgaatttggtatatttttattttatttttaaaatagtaaatatagttgaaaacaatattttttattgttatattattggtataaagaatttatcaactttgtgaatgaaagatctctgttagaaaatttaattaactatctttagaagattttctctccctaacgactaggctggaacccaagactgaaactgcgtcggttgccactgaaaccaacaatataaattgatttgtcttattttgcccttctcattcaccatatcatcacaaaataaggtgggatcggttagtttattccaaaaatagattgaaagggtcaatttatcttattttttgaccactcacgggccggtggatcatggatgtgcatgaaaattgactggctatgatttcttttttattcatttaattaaatgggtttaaagatgaaggtgatgtaaagtgatcaatgaagtatgttgacagataaatggattaataaaggtaaaatatgttttttatttttcataaatttgttaaatttaattttggtcttctaaaacaaattttgttcaattttcatctttctaatgtagaaatactttgattatgtctgataaaatctttgattaatttataagttttttgatcataataaatttgtttgataaacaaatttattttaataatttattttttataagctacttcgagtaggtttttgataagttatttgaagtattttataaaaaataaattagcttataacctattagtttttttttccttaatttatctttattattttatttgaaattttatttcacttttattcaattaaaaaactctcttatcttttttgtttgtcgtctcaaaaaatcttcatgtctaattttatatttttacatgtatatgacacacacgataattaatccatcaattattatcataatatcgttccttaagattataaaatttttgaattattactttttacaattatttgctgaattattctatgtttttcctaaaattatgtgacttaattcatttccttgcatgatttttattttatcaattcaattcgtataaatgaaaattttccttttgttaattaaaccctacttactattgaatatgtctttttatgttatttgatatttatcaattaacttatcagttaatcttaccaaataatctcaattacattagtcaccttctagctttttagcttagtttatagcttataagctaattttagcaaacataaccttcatattttatttttaaaattatatagttaacatttaataataattataaattgtcaaaaatacactttgattatcaaaaataaaaataaaataaaattgaaagaccaaaaatgaatttggcaaatgccactctagggattaactttgacattttctctcaatttcagaaattaaaatgactatttaaccatagcataattgtgaaactttaaagaaattgaagttggaagggtttactaaataaaaaaaaataatgtctaattaatattggcacatgtatatattaattactcgaaaagattaatccttgtggacagttgattaactttaaacatgcttaagggttattttgatagtgcgaaatggaatggaataaataagaaatatttgaattgaaagaatgtgttggaggtatgggaccacattctcttcctttttccgtgatttcttcccaatcaaacccatccttagatagatatagtctcataatcagtataaagaccaaaacttatgaaaatgcttaaaagtttcatctaagaattaaatatcctaactatggtgattggaaatatatattaattacaccaacgacaggaaaaaaaaactgctttagtgaaaatggccgttattaaaaaaatgatttgaaaatcaagaattttaatttttggggggagaatcaaagggcaccatcctgtgtccgtgggttgagataagagcatgttgtgaggcttagggggtgtttggtttacttgttttctgttttcattttcattgaaaatagaaaacggtgatgaaaatgtgtttgtttggatttcgaaaaacattttcagtcataatgaaaacaggaaataaccagaaaataaaaataataaattctgattttcagtgttttcagttgagaacagaaaccccatctcgagtaaattgaagaaattagaaaatctaaaaatacaaaaaagataataaatcaatatatcataaatttttagtatttttatttcatgaaaatagaaaataagaaatcaaatcatacatgttttcagaattctaatcttttgaaaatgaaaacaattttcagaaaaaatgaaaataagaaatgaaaacaaaaaataaaaatgtaaatcaaatacaccgttaagaattgaatttttttaattcactttcttttcataatcagtacttagactataataatctaaataaatttatttatcacttatttaataaattgataaccctcaacttctgaacctaacggaagtaactcttagcgagagatttttggattcacaagggttttaaaatgaagtctcttttttaaaaaataaaatgaatgttctaaactacaataactagttagggattaaataaaatcacggctgttaatttaaaagcaaaaatacaaataaacaaattgtgttacaaaatgaaaatatcatcttatgatagcaattaatttggaaggactagctctggaaccttcaattgtagcatggggcttgtttggagcacataaaggatgtaaccagtaagatagggagaggcacatatagcagctgaaaaaagccgcttctgtgcatcctttggtccccctttgaaccgggagtggaggggggtcattgcatcatctcaatcaaccacttttcttgttcctattaatgcaatgcctcattttttgacataatttcataaaagctaatttttttcaaatgcccatatttttttttcaacatcattggttacttgtgtgggttagcattctaaaaatctaaattaaatggcaacctagccacagatttttgtgtaattgaccaatagaaatattatactgttagtgaacattctagtcaaacaaacactagccaatgtttgattctaatgagatgaatgcaacaagctacgtgtggtaacaaattctccataaaacttatggatcaggaaaaggcttcaattccaacctcaagatctaggttggtgagaccttgcaggcactatgtggttgccacgtggcagccaagctaatcttcatcgtgtggatgttaaggactctacaactagtcttgaggatatgaatttctgaacactcaattataagtcccttccctattgtaatttctgccagcctcaaaaaatacttgctctacctctgccactttctgagtacaatacaacactgccatggctgcctcagtctccactattggagctgtcaacagagctccagtatgattttttttccttctctttgcactttcttgtccttagtttcttaactatttcttagatgaattttgtcatataaatgctcaaatcattattaatttgtgtaaggttatagatttatgtttgaaatatacatataacatatctatacatgcaaataagggttggcaccgtggagtaaaatcaattaatttttcacgttgaaagtgaagcaagtcattatcattatgggatttccttttttttttttgtagtttaggggtagccaatgtgcatccaaacatgattttgaatatctgaaagatatatgacactacctttatcctagtttcgttaagaattactccaagttctcatttcatatacatgtctatgacaagtttcttttttcgtgctagtaggagtcgctgagttttggtacacttcaaaaagtgtcaatttcaagaatatgatacatagagggaaaactcttatgatttttaactcctcatttatattgatagtaaactcaatataggttaataactgtgttatgtaatgtaataatcaatatatgtgatagcaaattagaaaagaatggtgttatttcttacttgagtcaagataaaatcaacagatttggatattagaagcatgtgacaagtgagtgtgaagttttaaaattagtttggacagtataaaagagtggttactgtgaatattgcagctgagcttgaacagctctgaagctggagattcggttcccagttcagccttctttggcacaagcttgaagaaggttactgcctcaagggtccctaacgtcaagatttcttctggaagtttcaaaatcgttgcggcggagaaagagattgatgagcaacaacagacaaacaaggacagatggaaaggtcttgcctatgatatttcagatgaccagcaagacatcacaagaggaaagggtatggttgattccctcttccaagctccacaggagactggaactcactatgcagtcatgagctcttttgagtaccttagcactggacttaaacagtgagtgacacttacaaaattagtgtttgcttaagttgaaaaattgaactcccttattttgatctaactaaaggtgttcatgatggtggtatttcaggtataacttggataacaacatggacggtttttacattgctcctgcttttatggacaagcttgttgttcacatcaccaagaacttcatgaccctgcctaacatcaaggcatgtaaattgcacacatgattcccttatacattgcctcaaaactatagttaatgaaacaggagacactcagtatttttcattgtttttacaggttcctctcattcttggtatttggggaggcaaaggtcaaggaaaatccttccaatgcgagctcgtctttgccaagatgggaatcaagtgagtaaatgttgtatatatattactactacataacagaacacatcagttatatacattactacattagcaactctagtgcattagaacttagtacttagcactcaatagctaaagtgtgtttcaatgacatctctaaatggaactttagttgaatataagaaaaaatatatttttagtctctcaaaatatcgaaaaattctatttagtccctataaatatataacttttttagtttctacatttttccacaaatgtgtgtttagtctctggcggctggcgagagactaaaactaggtttctctttataaatgtagggactaaaaagacattaaatttttaaagagacaggaaccaaattttgagatattttgatgcactaaaaacatatcttagccttgaatatattatttggtgattggtgctaacttatgatgttgtgatgacagccccatcatgatgagtgctggagagttggaaagtggaaatgcaggagagccagcaaagttgataaggcaaaggtaccgtgaagctgctgatataatcagcaaggggaagatgtgctgtctcttcataaacgatcttgatgcaggagctggtcgtcttggtggaaccactcaatacactgtgaacaaccagatggtgaatgccaccctcatgaacattgctgataaccccacaaatgttcagcttcctggcatgtacaacaagcaagagaacccccgtgtgccaatcatcgtcaccggtaacgatttctcaacactctatgctcctctcatccgtgatgggcgtatggagaagttctactgggcacctacaagggatgaccgagttggtgtctgcaagggaattttccgcactgacaatgttcctgaggatgaaattgtcaagcttgttgacaccttccctggccaatctattggtaagtttcctatgggaactcaagaactctctgcaatttgcaaatgatctaatatgtttcacaccccaaacatatataattaggacaaaacttagatgcagttcagtttcttcagtggttttagtattgttctctaataagaattggagtttttatttcgataactcattgatgtttaatacaaacttttattatgttgattaacaaagtgaaactctaattatgattgaaaaagagcatggaaaaacatttaagaaattacatctaagttttgtccatgtaattaaaacttacatattagtcccttaaattataagcaacaaccactttaatcctgatttttttaagggtaataatgtgttgccacctttacagattatttctttttatatctcttttcttatcacatcatatctcatctatcatgccaacactttttctctaggtgtcatttaccttttagatgtccttaattgcaataagactaatggtatgcaatttatcattgcagatttctttggtgcactcagggctagagtatatgacgatgaagtgaggaagtggatttctggtgttggtgttgacttgattgggaagaagcttgtgaactccaaggaaggacctccaacctttgaccaaccgaagatgactttgagcaagctcttggagtatggtaacatgcttgtccaagaacaagagaatgtgaagagagtacaactggcagacaagtacttgaacgaggctgctcttggtgatgctaatcaagatgccatcaaaagaggaactttctatggtttgttattttattctgaatttatataggatgtttgcatatagctcaattttcagtcaacttatacacacgtagaaattcagggaaagttttttcatgcaatttttttacattctcgggaatgttatttcaagaatataacatgaaaatattgttttcaggtatttaaaaaaatgtttggttacgttttaatattcttggaaatgatttttaaatttaaaataatttaaataaaaaagaagtttcatatgtttcaaacaataaattcttacattttcattggaatatcacttttctaccccttctcatgagaaaaatgttaaaaccatggtaaaaaaggaaattataatattttcagaaatcaataatacctagaaataatttttcaaaacttatgataaacataaaaacattatattctatttttacatttcccgagaatttgataaaattacgtgaaacaaatgtccctttaattttgatatagtaaaaaggtttccaatatgtctaataacatattgctcacttaaactattttaaaaatttgacaaaatataatcgaacaattacactatgtcaaaattaaactcattagaaagtatattttgatcaaataaaactaggtctaaactttttggttgttttattttctgcccaggcaaagcagcccagcaagtaaaaattcccgttcctgaaggttgtactgatccaaatgcctcaaacttcgacccaactgcaagaagtgatgatggaacctgcttatacacaccctgagaacctaagcattctccacttgaagagaagctatgacttgagataatcaaataatttatcttgttcatattggattgttatcctttataattttgagttacattgtctcactactaaaaaaaacacttgagataatcaattaatatttcctggtgttcattgtggttctgctgaactttgtggcttcaaataatagaaggtgttttactacaaaggttttaattaatgatgtcattattcatttaatattttcattgctgaattttgacaagggtttataaaatctcataggagacattggttcaattacatattgccattcatgaacaaaaatcaaaatattttaaaaaaattattattcctacatcggttataagaataatcgatgtagtattgctttatattctacatcggttatacataaaaccgatgtagaatgatcctaaatgaggtaccactcaactacgtttgaaggcggatggagatgtagaacgtcggacattctacatcagttatcaggagaatcgatgtagaatgccacaaccgatgtaaaatagtcagcattttaagacggatgtgtttcgagacccgttttcgaatccaactcattctaagacgacttgtcgaccgatgtagaaagaacacagagtttcaacatcgctggctacaatgatgcatgacaatcaatgtaaaaatatacttttaaccgatgtaaaaagataattttatagtagtgagatctatattttacaatgtgtacattaaagtcttgatacctttctctttctccctgtgcatttatttattgcttttctacatttaattttttccttgacacgatattaatgttatcctacctgttcattttaatcttgcatgcatacattgctctaaacatgagcgatcaatatgtcctggcaaattttagtttcttagcaaaagtagtttcatgtatcagcaatcacatgcatgaatgacctgttttaacccagtgtgttagttaaggcttttaagctaccttttagtttatttgatgagttttgaaagcttgtttgattaaaatcaaaccaacttaatgaagttttttttttataatttgtaacattgtttaaaacactacttcatgtaatattatttaaactcctaattaactttttttttttttatcttcactattatatcatacttttttaagagtatttctcacttctcaactacttttactttaatcaattaccttatcatccagtaaaaaaaaaaaacaattaccttatcattatcagcttttattttctaacttacaactaacatatttgctggtagcctagtatgaccaaaacaattattgaaatatgttcatgatgtttaattttattagaggaggaagtaaaagaatgtaagtgaattacacatttttcttaattaaataagtatgaaattcatcaaaagaaaattacatattaagaaaacatgaaagaattctcttcttggggttattcttgagctaacatattcgcaacaaacttcaactgcagttttgcaaccgtgttgtggccattacgggcaaatgcaagtcaattgtaatcacaattcgtgatcacagcataatgactgctaagtgctaacgcaattattgaaatattcaagcagcacccgatgatgagatgacgtacgtgatccaggtccattcagacatataaacgaacttagattctcaacgtgttaatcttaaaacttttacttattcttcggcataaacatatttttaaatcagtaaaatattttttattaaatgattctattaataaataaatatttaaagtaagtttataatttttaatttatcaaaaaattgtttaatacttaaaacatataataaattattattatatatatatataataataattttgataaatatatttattcttaattgtaataataaatataataatctataagatgattagtaaatatattttataatagtgttttcgatttaaagattacttttataataatttcaattttgataataatcttataagtaataagaagttgagataagaaaagtaaagattttgaaaagaaacgaggattgaaaaaaagcaaagggtttagaaaaaaagtaataaactaaaaaaatatatgaattcataaaagatgagaattaaataatgttcgatgacctatcaaatctgtaaccaattttaataata

SEQ ID NO: 41 - protein, 478 aa, Glycine max
MAASVSTIGAVNRAPLSLNSSEAGDSVPSSAFFGTSLKKVTASRVPNVKISSGSFKIVAAEKEIDEQQQTNKDRWKGLAYDISDDQQDITRGKGMVDSLFQAPQETGTHYAVMSSFEYLSTGLKQYNLDNNMDGFYIAPAFMDKLVVHITKNFMTLPNIKVPLILGIWGGKGQGKSFQCELVFAKMGINPIMMSAGELESGNAGEPAKLIRQRYREAADIISKGKMCCLFINDLDAGAGRLGGTTQYTVNNQMVNATLMNIADNPTNVQLPGMYNKQENPRVPIIVTGNDFSTLYAPLIRDGRMEKFYWAPTRDDRVGVCKGIFRTDNVPEDEIVKLVDTFPGQSIDFFGALRARVYDDEVRKWISGVGVDLIGKKLVNSKEGPPTFDQPKMTLSKLLEYGNMLVQEQENVKRVQLADKYLNEAALGDANQDAIKRGTFYGKAAQQVKIPVPEGCTDPNASNFDPTARSDDGTCLYTP

SEQ ID NO: 42 - DNA, 2084 bp, synthetic construct
  source 1..2084: mol_type = other DNA
ggctggggaggagatcattgaggtaaggttgttgtgcattgtttggttgagtgcaacgatttactgattaccatagctacttgtggatcttatattttcaaaagtgtatgcttggtcattaatcaaccaagcatgataagccccatcatctaccaccccttctataccatatacgcattttgcttatctacattgctactgttacactcaaacaagtctaacagataatacgttaagttaatgaacatttttagtaatattattataaggattggccaatgtaattgtgaagagagaagcatgctttaagctacccaacaaaatggataagagtttcagttgatatggttctcttgtttcttttccaataaaaaaccaactttaaaatataaaatttactgtaacaaaggaacaaagagttttcacttaatccatgaatgagaaaggatggtcacaaaatatgttaggttaatatggaatgagggcactgtgcaaactacacaaataatatcaattccaccaccatcacacatttacgttcttccaaggaagagataagataatggagcctccacgtgtcacctccacatggtacctaacaataaggctaccattcaaaattttcctcactcgtgtggcctatatgctgtaatgtcatcacttattcaatccaacggttgtaacttttcggcaaccaatcctctccatttcacaccattggattagtactacacaaatcacactattatatatagcaagtttgagcagaagcttggatatctggcagcagaaaaacaagtagttgagaactaagaagaaggaaatgggtgctttcactgagaagcaagaggctttggtgagtagctcattcgaagcattcaaggcaaacattcctcaatacagcgttgtgttctacacttcgatactggagaaagcacccgcagcaaaggacttgttctcgtttctatctaatggagtagatcctagtaatcctaagctcacgggccatgctgaaaagctttttggattggtgcgtgactcagctggtcaacttaaagcaaatggaacagtagtggctgatgccgcacttggttctatccatgcccaaaaagcaatcactgatcctcagttcgtggtggttaaagaagcactgctgaaaacaataaaggaggcagttggggacaaatggagtgatgaattgagcagtgcttgggaagtagcctatgatgaattggcagcagctattaagaaggcattttaggtcattgaaaaatttgcaaagcatctgtagccaccccactttgtttgttgtacttaaactacattcccatttgtttttgctttatgagatttcatcatcctgtatttttggtttctgttttcggacttcaatggaaattaatggatgagaactaatgaataagctattgtgttgtgttgctttgtttccaaataacttcaagaacccattgtccttgcatttccatcttgtgggttgaaattagtctcttctaaatttaagttaattgtgtcactaaatgatggttaacaaaggaattctttctgcgttattcaaagtacacagcatagtttagttgagcttggccttactttgtcacaaatctgatgtatgaatgttctttatactctggttttccaagaaatgaagttcaagagatagtttgtaaacatgttgctactgaactttcaaaaccaaacatgagccccataattcttggaaacacagatattttgtatggtatatagcaacaaaatctactaaggaactgctaccaaaccgttttgttatttatgtgcaaagaaagcgatatcaaattcaaatttttgttggtttcagaaacttttctttccaagcaataaccatcaactgcaactaaaccgttttgtttgttccatttctcatctcctcactgcaattttcaaattatgatttggataacacataaaaacacaatcagatttggtcatgctctggttctaccaaatacttttttttttctagaattgcctgaatctgatccttagctgacaattaatctcgtttcaatgacttagaagcctgg

SEQ ID NO: 43 - DNA, 2084 bp, synthetic construct
  source 1..2084: mol_type = other DNA
gaacagtgtgatgaaaatgaagggagattcaatataattaactatcatcaaacagcttcctggtatatgaacatgcaggctttttctttgtaatctgaggggtatttcttatgcctcatatgaatattattctctctttgccgtttcagaaaaaaaaaaaaaaacttaagaatatacatgcatatatatatatatatatatatatatatacatttatcttaaatatatattttaaagtaattattataaaaataaattattttataataaagaaccatctatgatcaaataaatgatatatataacttgaatttgcctgctgttagataatttaacaactaattaactgtaagtaatctcagttccttaccgctagaaaagttaacatttaatgttctgacacattcagaatttgttatgtacgtcctttacattacacccaatactttatctttcaaaatgagtttctcacgttaaaaaaaaaaaaaaaaaagttgcatccaacagactttaatttgtacaaaatgcgttttgattattaaattctaaaaaattaacctacctgattgttttttttaaaaaaaaaatatgttcaaccaaattctattgaagattaaaaaaaaagtagacaaaaaacataaaagaaaataacattctggactagtaaaatgaattattggaccgtaaaaattagcttattacagttcagaaatattttatcctctatgcagtaggcagaggtattgtctatttaaagaagcaaatcctcgatgaggaaagtgcacaagtgagtttgttgatcgagataactagaaacagtgtgatgggtgctttcactgagaagcaagaggctttggtgagtagctcattcgaagcattcaaggcaaacattcctcaatacagcgttgtgttctacacttcgatactggagaaagcacccgcagcaaaggacttgttctcgtttctatctaatggagtagatcctagtaatcctaagctcacgggccatgctgaaaagctttttggattggtgcgtgactcagctggtcaacttaaagcaaatggaacagtagtggctgatgccgcacttggttctatccatgcccaaaaagcaatcactgatcctcagttcgtggtggttaaagaagcactgctgaaaacaataaaggaggcagttggggacaaatggagtgatgaattgagcagtgcttgggaagtagcctatgatgaattggcagcagctattaagaaggcattttagtaaattatgcgcccttgttccatactttgatcttttcttttcttttctttttctttttaagtcaaagtaattcattattatcaaactaatgaaaaatactaaccaatatcctaagagaattggttaaagaatttaaaataataatatttttattaagagacacaaaattatgttatttataaattttttttattttcatataacttacacaattaatatttttgtcttttagttttataacaaatgtcttaaaaacactaattaaaaagattctttaaaaaatagtcttaagaaaatctgaggaacttagctaggttttacaaactaatataaaatgtgaattaaatatattttttccatttaataatttagtattttttttcatccattacattattttgaataataaaaaatattataaagtgttttaaggatgaaaaataaaagaatatattttaaataaaataaaaaagaaatttgcaggaaaaaaatatctaaaagataaagattttataaaatcgagatcgggaaagaagtgcacaaaatcgacgtcgtgaaagaagtgcacaaaatcgacgtcgtctattacaccatataattacactgtgaaccgacagttggtattaacacaatggcccaatagagtagaccgagaaataattatctgacaaatatactgttcactaaaaacaaaaaaatgatgagaaagcctgaaaattacactatatatatatatatatatatatatatatatatatatatatatatatatatatatatatgaaatataataactttatgataagatgcgttcaaatagggg

SEQ ID NO: 44 - DNA, 2084 bp, synthetic construct
  source 1..2084: mol_type = other DNA
gagaaattaaagtgggaaaaaggggtccatgaatgtaattaataggactcaatcaccttcatcaaattataatccgactcaccaaaattaatttcttgtatctacccaataataattgagaaacaaatcttatattaataatataaaataattttacatattattgaatcataaattatcttaaaattaaataatgttataaaattattttacgttctaagtggcattaaactcataataattcatgttaaatttatcttcatctgaaaatactctaagattctaagttaacataatttcatgaaaattgaaaattggatatgttggctactatgtggggtgaacagtaccatctttacgtaaataaaacaccacctcaaatattatggtagttgttctttaaaaaatactgataatattatcattggttatcaagattgaagagcactgaaatgacaataaccaatgctttgaatttgaataaacgacaaaagttctcctcacaggcttgtgtgtgtggcaataacatctccttaatactggtggagatagaaaaagcttgaataccaaccgcaaagagtaggactaagagtgacaaaaaaccttgcaggcacattatgttgccacgtgtcggctgatcaatcttctatgtgtggatgtaaggcttctatgaccaatccttttgtgtacactcactccaccaagcaactactataagtcacagcctcattcatggtcacaagccacttagcactgagtcttgcaattgaagttgaagtcttgcccttctctcttcaactccaaacacaagacatcatcatccatgggtgctttcactgagaagcaagaggctttggtgagtagctcattcgaagcattcaaggcaaacattcctcaatacagcgttgtgttctacacttcgatactggagaaagcacccgcagcaaaggacttgttctcgtttctatctaatggagtagatcctagtaatcctaagctcacgggccatgctgaaaagctttttggattggtgcgtgactcagctggtcaacttaaagcaaatggaacagtagtggctgatgccgcacttggttctatccatgcccaaaaagcaatcactgatcctcagttcgtggtggttaaagaagcactgctgaaaacaataaaggaggcagttggggacaaatggagtgatgaattgagcagtgcttgggaagtagcctatgatgaattggcagcagctattaagaaggcattttagattggaggtttgtataatgtttctctaacttgcaaaaataatgttaggttcaaagtataacttggtctaaaaatgttgaatttcttttttttttttttctgtgcaggcaaagcaaacttggcaagtaaatatttctgttcctgaaggttgtgctgatccaagtgctagaagttgtgatgggacttgcgtgtaacattcttagaattctcaatgtcttaggtgcaaagagaagatgccaagtttgagatgattttactatatttttcatattggactgtcttcctctataattttgagttaaagtgtccacttaaattgaatttggtgccttttccttttgtcctaaattcttgattttttatgctttaaatttatatcttttgtttgacgtacaaatattagtacatacgtagcgttatatcttcattctcgaagtgctaaggatccgaagaacttggagagaaaggtcgagttcagagcttaataatgaatggcaagctgccaccatatcataaacaccacaatcataatcattaaaaattgtttcttatatacaaggataaatagtcacttttgtccctcaatatataatttgctgacaaatacactcttgaaagatgaaaatacaaaatttagtctctgaaagtgtaaaaaatgtgacaaatatatcataccgttaatttttgtctgtcatcgttaataaagtcgcatacgtggcacaaaatgataaatttgtcactgaaataatagccaacatgaattgccagcatagaggcatatttgtcataatattttttgacgctttcttccaatcttgaattgg

SEQ ID NO: 45 - protein, 20 aa, Glycine max
KANGTVVADAALGSIHAQKA

SEQ ID NO: 46 - protein, 13 aa, Glycine max
KAITDPQFVVVKE

SEQ ID NO: 47 - DNA, 100 bp, Glycine max
  source 1..100: mol_type = genomic DNA
atgccccatttttctctctcccctctctcctgtgcaccttctcctctcccacctcgccgccgaatctttctcgctcctccccgaaccgccgcatcttccg

SEQ ID NO: 48 - protein, 83 aa, Glycine max
MPHFSLSPLSCAPSPLPPRRRIFLAPPRTAASSATDAASSSTSSSASNFAPKVVVTRERGKNAKLIAALAKHEINCLELPLIE

SEQ ID NO: 49 - DNA, 1413 bp, Glycine max
  source 1..1413: mol_type = genomic DNA
atggctgtttcggctatcacaggagcgaggctaaccctagggatagggttggcgatacctctttcctctcccacgcgctctcgaaccgtcgcaatggccgtatccgtcgaccccaagaccgacaacaaactcactcttaccaagtccgaggaagctttcgctgctgccaaggagctgatgcctggaggtgtcaactccccagttcgtgccttcaaatccgtgggtggtcaaccaattgtgattgattcagtcaaagggtctcgtatgtgggacatcgacggcaatgagtacattgactacgtcggttcttggggtcccgcaatcattggtcacgctgatgatcaagtgctttcagctctggttgaaaccatgaagaaaggaactagctttggtgcaccctgtctgctggaaaacactttggcagagctggttatcaatgcggtccccagcattgaaatggttcgctttgtcaattcaggcaccgaagcttgcatgggtgcactacgtctcgcccgagcttataccggaagagagaagatcatcaagtttgagggctgttaccatggccatgctgatccttttcttgttaaggcaggtagtggagttgccaccttgggacttcctgattctcccggtgtccccaaagctgccacttttgaaacccttacagccccctacaatgacactgccgccgttgagaagctctttgaggctaacaaaggagaaatcgctgctgttttcctcgaacctgttgttggaaacgctggtttcattgttcctaaacccgattttcataatttcttgcgcaagatcaccaaggagaacaatacccttcttgtgtttgatgaagttatgactgggtttcgtttgtcatacggaggtgctcaagagtattttggcataactcctgatataacaactctaggaaagatcattggtggaggtctgccggtgggggcttatggagggaggagggatattatggagaaggtggcaccagctggcccaatgtatcaggctgggaccttgagtgggaaccctttggccatgactgcaggaatacagaccctgcagcgtattaaggagccaggaacttatgagtacttggacaaaatcaccggtgagcttgttcagggcattattgaagctgggaagagggcaggccatgcaatatgtggtggtcatataagggggatgtttgggtttttcttcacagaaggaccagtgtataattttgcagatgccaaaaagagtgatacggacaagtttgctaggttcttttggggaatgttggcggaaggtgtctatttggcaccttcccagtttgaggctggctttaccagcttggcacatacctctgatgacataaaaaagacaatagccgctgctgagaaggttttcagggagatctga

SEQ ID NO: 50 - protein, 470 aa, Glycine max
MAVSAITGARLTLGIGLAIPLSSPTRSRTVAMAVSVDPKTDNKLTLTKSEEAFAAAKELMPGGVNSPVRAFKSVGGQPIVIDSVKGSRMWDIDGNEYIDYVGSWGPAIIGHADDQVLSALVETMKKGTSFGAPCLLENTLAELVINAVPSIEMVRFVNSGTEACMGALRLARAYTGREKIIKFEGCYHGHADPFLVKAGSGVATLGLPDSPGVPKAATFETLTAPYNDTAAVEKLFEANKGEIAAVFLEPVVGNAGFIVPKPDFHNFLRKITKENNTLLVFDEVMTGFRLSYGGAQEYFGITPDITTLGKIIGGGLPVGAYGGRRDIMEKVAPAGPMYQAGTLSGNPLAMTAGIQTLQRIKEPGTYEYLDKITGELVQGIIEAGKRAGHAICGGHIRGMFGFFFTEGPVYNFADAKKSDTDKFARFFWGMLAEGVYLAPSQFEAGFTSLAHTSDDIKKTIAAAEKVFREI

SEQ ID NO: 51 - DNA, 918 bp, Glycine max
  source 1..918: mol_type = genomic DNA
atgcttcttcaaacgaggcaatttggcacagcgtgtccatgggtcagactcacacccgcaagcaaaagaaatagctcagtctcattgagaaggagcaattgtggcccaaaatgttcggtgtcccaattggaattgaagagtgagagtgagagcaaaacgaagccgtatccagcagaggcatcgaggaccataatggagttggctaaggtgggcacgctgtgtacgttgacccaagagggtttgccccttggtactggggttcgattcgcggttgaccctgaacatggcactcccttgttctgcttcaattccactgacaacactaacatcccctcttctcttcatgttcagtttgaacaatttggattgcgcactcctcagtgcactattcaaggaacccttaccaaaccacaggatccaaagcgtcttgtttctgtgtggaggaaaaggtttggagaagaagttgatcgagattttatttacattattgccgtggatcgggtactccaattggaagacattcaggaggatggcatatgggtcacctcttcagattacaaaaatgctcaacccgatcctcttcgagactctgcacacaacttagtcactgaaatcaacaccaacaatatggaagacattactcgcttttgcaatgtctacgttgatttggatttcctggtctccgaggcaaagatgctatgggttgatcgcttgggctttgacatgcgtttgtcttcccctcacaaaggcgtgtttgacgtccgcattcctttccccagagaagtcaccgatgagaaaggtgccaagtcgacgtttaattgtatgtcacaactcgcttgggaggtagaaagaaacttccaacatccagacttttcaaaggttaaagagttgaagccagtcaagtccccttttctgtaa

SEQ ID NO: 52 - protein, 305 aa, Glycine max
MLLQTRQFGTACPWVRLTPASKRNSSVSLRRSNCGPKCSVSQLELKSESESKTKPYPAEASRTIMELAKVGTLCTLTQEGLPLGTGVRFAVDPEHGTPLFCFNSTDNTNIPSSLHVQFEQFGLRTPQCTIQGTLTKPQDPKRLVSVWRKRFGEEVDRDFIYIIAVDRVLQLEDIQEDGIWVTSSDYKNAQPDPLRDSAHNLVTEINTNNMEDITRFCNVYVDLDFLVSEAKMLWVDRLGFDMRLSSPHKGVFDVRIPFPREVTDEKGAKSTFNCMSQLAWEVERNFQHPDFSKVKELKPVKSPFL

SEQ ID NO: 53 - protein, 55 aa, Glycine max
MASSMISSPAVTTVNRAGAGMVAPFTGLKSMAGFPTRKTNNDITSIASNGGRVQC

SEQ ID NO: 54 - protein, 4 aa, Glycine max
KDEL